(19) United States
(12) Patent Application Publication
(10) Pub. No.: US 2007/0232644 A1
(43) Pub. Date: Oct. 4, 2007

(54) Inhibitors of the Hiv Integrase Enzyme

(75) Inventors: Klaus Ruprecht Dress, San Diego, CA (US); Jon Edward Kuehler, San Diego, CA (US); Michael Bruno Plewe, San Diego, CA (US); Anle Yang, San Diego, CA (US); Junhu Zhang, San Diego, CA (US)

Correspondence Address: PFIZER INC, 150 EAST 42ND STREET, 5TH FLOOR - STOP 49, NEW YORK, NY, 10017-5612, US

(73) Assignee: PFIZER INC.

(21) Appl. No.: 11/574,869

(22) PCT Filed: Aug. 26, 2005
(86) PCT No.: PCT/IB05/02967

§ 371 Date: Mar. 7, 2007

Related U.S. Application Data
(60) Provisional application No. 60/607,791, filed on Sep. 7, 2004.

(51) Int. Cl. A61K 31/44; C07D 471/02

(52) U.S. Cl. 514/300; 546/113

(57) ABSTRACT

The present invention relates to compounds of formula (I), 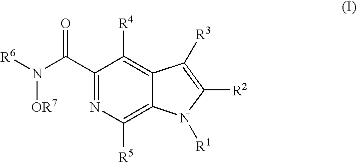 or a pharmaceutically acceptable salt or solvate thereof, pharmaceutical compositions comprising compounds of formula (I), and their methods of use in treating HIV-infected mammals.

### FIELD OF THE INVENTION

The present invention is directed to compounds, and pharmaceutically acceptable salts or solvates thereof, their synthesis, and their use as modulators or inhibitors of the human immunodeficiency virus (“HIV”) integrase enzyme. The compounds of the present invention are useful for modulating (e.g. inhibiting) an enzyme activity of HIV integrase enzyme and for treating diseases or conditions mediated by HIV, such as for example, acquired immunodeficiency syndrome (“AIDS”), and AIDS related complex (“ARC”).

### BACKGROUND OF THE INVENTION

The retrovirus designated “human immunodeficiency virus” or “HIV” is the etiological agent of a complex disease that progressively destroys the immune system. The disease is known as acquired immune deficiency syndrome or AIDS. AIDS and other HIV-caused diseases are difficult to treat due to the ability of HIV to rapidly replicate, mutate and acquire resistance to drugs. In order to slow the proliferation of the virus after infection, treatment of AIDS and other HIV-caused diseases has focused on inhibiting HIV replication.

Since HIV is a retrovirus, and thus, encodes a positive-sense RNA strand, its mechanism of replication is based on the conversion of viral RNA to viral DNA, and subsequent insertion of the viral DNA into the host cell genome. HIV replication relies on three constitutive HIV encoded enzymes: reverse transcriptase (RT), protease and integrase.

Upon infection with HIV, the retroviral core particles bind to specific cellular receptors and gain entry into the host cell cytoplasm. Once inside the cytoplasm, viral RT catalyzes the reverse transcription of viral ssRNA to form viral RNA-DNA hybrids. The RNA strand from the hybrid is then partially degraded and a second DNA strand is synthesized resulting in viral dsDNA. Integrase, aided by viral and cellular proteins, then transports the viral dsDNA into the host cell nucleus as a component of the pre-integration complex (PIC). In addition, integrase provides the permanent insertion, i.e., integration, of the viral dsDNA to the host cell genome, which, in turn, provides viral access to the host cellular machinery for gene expression. Following integration, transcription and translation produce viral precursor proteins.

A key step in HIV replication, insertion of the viral dsDNA into the host cell genome, is believed to be mediated by integrase in at least three, and possibly, four, steps: (1) assembly of proviral DNA; (2) 3′-end processing causing assembly of the PIC; (3) 3′-end joining or DNA strand transfer, i.e., integration; and (4) gap filling, a repair function. See, e.g., Goldgur, Y. et al., PNAS 96(23): 13040-13043 (November 1999); Sayasith, K. et al., Expert Opin. Ther. Targets 5(4): 443-464 (2001); Young, S. D., Curr. Opin. Drug Disc. & Devel. 4(4): 402-410 (2001); Wai, J. S. et al., J. Med. Chem. 43(26): 4923-4926 (2000); Debyser, Z. et al., Assays for the Evaluation of HIV-1 Integrase Inhibitors, from Methods in Molecular Biology 160:139-155, Schein, C. H. (ed.), Humana Press Inc., Totowa, N.J. (2001); and Hazuda, D. et al., Drug Design and Disc. 13:17-24 (1997).

Currently, AIDS and other HIV-caused disease are treated with an “HIV cocktail” containing multiple drugs including RT and protease inhibitors. However, numerous side effects and the rapid emergence of drug resistance limit the ability of the RT and protease inhibitors to safely and effectively treat AIDS and other HIV-caused diseases. In view of the shortcomings of RT and protease inhibitors, there is a need for another mechanism through which HIV replication can be inhibited. Integration, and thus integrase, a virally encoded enzyme with no mammalian counterpart, is a logical alternative. See, e.g., Wai, J. S. et al., J. Med. Chem. 43:4923-4926 (2000); Grobler, J. et al., PNAS 99: 6661-6666 (2002); Pais, G. C. G. et al., J. Med. Chem. 45: 3184-3194 (2002); Young, S. D., Curr. Opin. Drug Disc. & Devel. 4(4): 402-410 (2001); Godwin, C. G. et al., J. Med. Chem. 45: 3184-3194 (2002); Young, S. D. et al., “L-870, 810: Discovery of a Potent HIV Integrase Inhibitor with Potential Clinical Utility,” Poster presented at the XIV International AIDS Conference, Barcelona (Jul. 7-12, 2002); and WO 02/070491.

It has been suggested that for an integrase inhibitor to function, it should inhibit the strand transfer integrase function. See, e.g., Young, S. D., Curr. Opin. Drug Disc. & Devel. 4(4): 402-410 (2001). Thus, there is a need for HIV inhibitors, specifically, integrase inhibitors, and, more specifically, strand transfer inhibitors, to treat AIDS and other HIV-caused diseases.

### SUMMARY OF THE INVENTION

In one aspect of the present invention are compounds of formula (I), 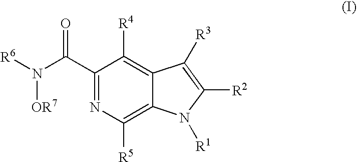
wherein:

R¹ is hydrogen, C₁-C₈ alkyl, C₂-C₈ alkenyl, or C₁-C₈ heteroalkyl, wherein said C₁-C₈ alkyl, C₂-C₈ alkenyl, or C₁-C₈ heteroalkyl groups may be optionally substituted with one or more substituent independently selected from:
halo, OR^{15a}, —N(R^{15a}R^{15b}), —C(O)N(R^{15a}R^{15b}), —NR^{15a}C(O)N(R^{15a}R^{15b}), —NR^{15a}C(O)R^{15a}, —NR^{15a}C(NR^{15a})N(R^{15a}R^{15b}), —SR^{15a}, —S(O)R^{15a}, —S(O)₂R^{15a}, —S(O)₂N(R^{15a}R^{15b}), C₁-C₈ alkyl, C₆-C₁₄ aryl, C₃-C₈ cycloalkyl, and C₂-C₉ heteroaryl, wherein said C₁-C₈ alkyl, C₆-C₁₄ aryl, C₃-C₈ cycloalkyl, and C₂-C₉ heteroaryl groups are optionally substituted with one or more substituent independently selected from halo, —C(R^{15a}R^{15b}R^{15c}), —OH, and C₁-C₈ alkoxy;

R² is hydrogen;

R³ is —(CR⁸R⁹)ₜNR¹⁰R¹¹ or —(CR⁸R⁹)ₜN(R^{15a}R¹⁶);

R⁴ is hydrogen, halo, C₁-C₈ alkyl, OR^{15a}, NR^{15a}R^{15b}, C₁-C₈ heteroalkyl, C₂-C₈ alkenyl, or C₂-C₈ alkynyl, wherein said C₂-C₈ alkenyl or C₂-C₈ alkynyl are optionally substituted with one or more R¹² group;

R⁵ is hydrogen;

R⁶ is hydrogen, C₁-C₈ alkyl, C₁-C₈ heteroalkyl, or C₂-C₈ alkenyl, wherein said C₁-C₈ alkyl and C₂-C₈ alkenyl groups are optionally substituted with one or more C₆-C₁₄ aryl or —OR^{15a} group;

R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with one or more C₃-C₈ cycloalkyl or C₆-C₁₄ aryl group;

each R⁸ and R⁹, which may be the same or different, are independently selected from hydrogen and C₁-C₈ alkyl;

R¹⁰ and R¹¹, together with the nitrogen atom to which they are attached, form a C₂-C₉ heterocyclyl group, substituted with at least one R¹³ group;

each R¹² is independently selected from —OR^{15a}, halo, C₆-C₁₄ aryl, C₂-C₉ heteroaryl, C₁-C₈ heteroalkyl, C₃-C₈ cycloalkyl, C₂-C₉ heterocyclyl, and —C(R^{15a}R^{15b}R^{15c});

R¹³ is selected from —(CR⁸R⁹)ₜ—OR^{15a}, —(CR⁸R⁹)ₜ—C(O)R^{15a}, —(CR⁸R⁹)ₜ—C(O)NR^{15a}R^{15b}, —(CR⁸R⁹)ₜ—S—R^{15a}, —(CR⁸R⁹)ₜ—S(O)—R^{15a}, —(CR⁸R⁹)ₜ—S(O)₂—R^{15a}, —(CR⁸R⁹)ₜ—(C₂-C₉ heterocyclyl), —(CR⁸R⁹)ₜ—(C₆-C₁₄ aryl) and —(CR⁸R⁹)ₜ—(C₂-C₉ heteroaryl);

each R^{15a}, R^{15b}, and R^{15c}, which may be the same or different, is independently selected from hydrogen and C₁-C₈ alkyl;

R¹⁶ is —(CH₂)ₘ—(C₂-C₉ heterocyclyl) or —(CH₂)ₘ—(C₃-C₈ cycloalkyl), wherein said C₂-C₉ heterocyclyl and C₃-C₈ cycloalkyl groups are substituted with one or more groups selected from C₃-C₈ cycloalkyl and —(CR⁸R⁹)ₜ—OR^{15a};

each m is independently selected from 0, 1, and 2; and

each t is independently selected from 0, 1, 2, and 3; or

pharmaceutically acceptable salts or solvates thereof.

Further provided herein are compounds of formula (I), wherein:

R¹ is hydrogen, C₁-C₈ alkyl, C₂-C₈ alkenyl, or C₁-C₈ heteroalkyl, wherein said C₁-C₈ alkyl, C₂-C₈ alkenyl, or C₁-C₈ heteroalkyl groups may be optionally substituted with one or more substituent independently selected from:
halo, —OR^{15a}, —N(R^{15a}R^{15b}), —C(O)N(R^{15a}R^{15b}), —NR^{15a}C(O)N(R^{15a}R^{15b}), —NR^{15a}C(O)R^{15a}, —NR^{15a}C(NR^{15a})N(R^{15a}R^{15b}), —SR^{15a}, —S(O)R^{15a}, —S(O)₂R^{15a}, —S(O)₂N(R^{15a}R^{15b}), C₁-C₈ alkyl, C₆-C₁₄ aryl, C₃-C₈ cycloalkyl, and C₂-C₉ heteroaryl, wherein said C₁-C₈ alkyl, C₆-C₁₄ aryl, C₃-C₈ cycloalkyl, and C₂-C₉ heteroaryl groups are optionally substituted with one or more substituent independently selected from halo, —C(R^{15a}R^{15b}R^{15c}), —OH, and C₁-C₈ alkoxy;

R² is hydrogen;

R³ is —(CR⁸R⁹)ₜNR¹⁰R¹¹;

R⁴ is hydrogen, halo, C₁-C₈ alkyl, —OR^{15a}, —NR^{15a}R^{15b}, C₁-C₈ heteroalkyl, C₂-C₈ alkenyl, or C₂-C₈ alkynyl, wherein said C₂-C₈ alkenyl or C₂-C₈ alkynyl are optionally substituted with one or more R¹² group;

R⁵ is hydrogen;

R⁶ is hydrogen, C₁-C₈ alkyl, C₁-C₈ heteroalkyl, or C₂-C₈ alkenyl, wherein said C₁-C₈ alkyl and C₂-C₈ alkenyl groups are optionally substituted with one or more C₆-C₁₄ aryl or —OR^{15a} group;

R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with one or more C₃-C₈ cycloalkyl or C₆-C₁₄ aryl group;

each R⁸ and R⁹, which may be the same or different, are independently selected from hydrogen and C₁-C₈ alkyl;

R¹⁰ and R¹¹, together with the nitrogen atom to which they are attached, form a C₂-C₉ heterocyclyl group, substituted with at least one R¹³ group;

each R¹² is independently selected from —OR^{15a}, halo, C₆-C₁₄ aryl, C₂-C₉ heteroaryl, C₁-C₈ heteroalkyl, C₃-C₈ cycloalkyl, C₂-C₉ heterocyclyl, and —C(R^{15a}R^{15b}R^{15c});

R¹³ is selected from —(CR⁸R⁹)ₜ—OR^{15a}, —(CR⁸R⁹)ₜ—C(O)R^{15a}, —(CR⁸R⁹)ₜ—C(O)NR^{15a}R^{15b}, —(CR⁸R⁹)ₜ—S—R^{15a}, —(CR⁸R⁹)ₜ—S(O)—R^{15a}, —(CR⁸R⁹)ₜ—S(O)₂—R^{15a}, —(CR⁸R⁹)ₜ—(C₂-C₉ heterocyclyl), —(CR⁸R⁹)ₜ—(C₆-C₁₄ aryl) and —(CR⁸R⁹)ₜ—(C₂-C₉ heteroaryl);

each R^{15a}, R^{15b}, and R^{15c}, which may be the same or different, is independently selected from hydrogen and C₁-C₈ alkyl; and

each t is independently selected from 0, 1, 2, and 3; or

pharmaceutically acceptable salts or solvates thereof.

In yet another aspect are provided compounds of formula (I), wherein:

R¹ is C₁-C₈ alkyl substituted with C₆-C₁₄ aryl, wherein said C₆-C₁₄ aryl group is optionally substituted with one or more substituent independently selected from halo, —C(R^{15a}R^{15b}R^{15c}), —OH, and C₁-C₈ alkoxy;

R² is hydrogen;

R³ is —(CR⁸R⁹)ₜNR¹⁰R¹¹;

R⁴ is hydrogen, halo, C₁-C₈ alkyl, —OR^{15a}, NR^{15a}R^{15b}, C₁-C₈ heteroalkyl, C₂-C₈ alkenyl, or C₂-C₈ alkynyl, wherein said C₂-C₈ alkenyl or C₂-C₈ alkynyl are optionally substituted with one or more R¹² group;

R⁵ is hydrogen;

R⁶ is hydrogen, C₁-C₈ alkyl, C₁-C₈ heteroalkyl, or C₂-C₈ alkenyl, wherein said C₁-C₈ alkyl and C₂-C₈ alkenyl groups are optionally substituted with one or more C₆-C₁₄ aryl or —OR^{15a} group;

R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with one or more C₃-C₈ cycloalkyl or C₆-C₁₄ aryl group;

each R⁸ and R⁹, which may be the same or different, are independently selected from hydrogen and C₁-C₈ alkyl;

R¹⁰ and R¹¹, together with the nitrogen atom to which they are attached, form a C₂-C₉ heterocyclyl group, substituted with at least one R¹³ group;

each R¹² is independently selected from —OR^{15a}, halo, C₆-C₁₄ aryl, C₂-C₉ heteroaryl, C₁-C₈ heteroalkyl, C₃-C₈ cycloalkyl, C₂-C₉ heterocyclyl, and —C(R^{15a}R^{15b}R^{15c});

R¹³ is selected from —(CR⁸R⁹)ₜ—OR^{15a}, —(CR⁸R⁹)ₜ—C(O)R^{15a}, —(CR⁸R⁹)ₜ—C(O)NR^{15a}R^{15b}, —(CR⁸R⁹)ₜ—S—R^{15a}, —(CR⁸R⁹)ₜ—S(O)—R^{15a}, —(CR⁸R⁹)ₜ—S(O)₂—R^{15a}, —(CR⁸R⁹)ₜ—(C₂-C₉ heterocyclyl), —(CR⁸R⁹)ₜ—(C₆-C₁₄ aryl) and —(CR⁸R⁹)ₜ—(C₂-C₉ heteroaryl);

each R^{15a}, R^{15b}, and R^{15c}, which may be the same or different, is independently selected from hydrogen and C₁-C₈ alkyl; and

each t is independently selected from 0, 1, 2, and 3; or

pharmaceutically acceptable salts or solvates thereof.

In still another aspect are compounds of formula (I), wherein:

R¹ is C₁-C₈ alkyl substituted with C₆-C₁₄ aryl, wherein said C₆-C₁₄ aryl group is optionally substituted with one or more substituent independently selected from halo, —C(R^{15a}R^{15b}R^{15c}), —OH, and C₁-C₈ alkoxy;

R² is hydrogen;

R³ is —(CR⁸R⁹)ₜNR¹⁰R¹¹;

R⁴ is hydrogen;

R⁵ is hydrogen;

R⁶ is hydrogen, C₁-C₈ alkyl, C₁-C₈ heteroalkyl, or C₂-C₈ alkenyl, wherein said C₁-C₈ alkyl and C₂-C₈ alkenyl groups are optionally substituted with one or more C₆-C₁₄ aryl or —OR^{15a} group;

R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with one or more C₃-C₈ cycloalkyl or C₆-C₁₄ aryl group;

each R⁸ and R⁹, which may be the same or different, are independently selected from hydrogen and C₁-C₈ alkyl;

R¹⁰ and R¹¹, together with the nitrogen atom to which they are attached, form a C₂-C₉ heterocyclyl group, substituted with at least one R¹³ group;

R¹³ is selected from —(CR⁸R⁹)ₜ—OR^{15a}, —(CR⁸R⁹)ₜ—C(O)R^{15a}, —(CR⁸R⁹)ₜ—C(O)NR^{15a}R^{15b}, —(CR⁸R⁹)ₜ—S—R^{15a}, —(CR⁸R⁹)ₜ—S(O)—R^{15a}, —(CR⁸R⁹)ₜ—S(O)₂—R^{15a}, —(CR⁸R⁹)ₜ—(C₂-C₉ heterocyclyl), —(CR⁸R⁹)ₜ—(C₆-C₁₄ aryl) and —(CR⁸R⁹)ₜ—(C₂-C₉ heteroaryl);

each R^{15a}, R^{15b}, and R^{15c}, which may be the same or different, is independently selected from hydrogen and C₁-C₈ alkyl; and

each t is independently selected from 0, 1, 2, and 3; or

pharmaceutically acceptable salts or solvates thereof.

In yet another aspect are provided compounds of formula (I), wherein:

R¹ is C₁-C₈ alkyl substituted with C₆-C₁₄ aryl, wherein said C₆-C₁₄ aryl group is substituted with one or more halo;

R² is hydrogen;

R³ is —(CH₂)NR¹⁰R¹¹;

R⁴ is hydrogen;

R⁵ is hydrogen;

R⁶ is hydrogen or C₁-C₈ alkyl;

R⁷ is hydrogen or C₁-C₈ alkyl;

each R⁸ and R⁹, which may be the same or different, are independently selected from hydrogen and C₁-C₈ alkyl;

R¹⁰ and R¹¹, together with the nitrogen atom to which they are attached, form a C₂-C₉ heterocyclyl group, substituted with at least one R¹³ group;

R¹³ is selected from —(CR⁸R⁹)ₜ—OR^{15a}, —(CR⁸R⁹)ₜ—C(O)R^{15a}, —(CR⁸R⁹)ₜ—C(O)NR^{15a}R^{15b}, —(CR⁸R⁹)ₜ—S—R^{15a}, —(CR⁸R⁹)ₜ—S(O)—R^{15a}, —(CR⁸R⁹)ₜ—S(O)₂—R^{15a}, —(CR⁸R⁹)ₜ—(C₂-C₉ heterocyclyl), —(CR⁸R⁹)ₜ—(C₆-C₁₄ aryl) and —(CR⁸R⁹)ₜ—(C₂-C₉ heteroaryl);

each R^{15a}, R^{15b}, and R^{15c}, which may be the same or different, is independently selected from hydrogen and C₁-C₈ alkyl; and

each t is independently selected from 0, 1, 2, and 3; or

pharmaceutically acceptable salts or solvates thereof.

Further still are provided compounds of formula (I), wherein:

R¹ is C₁-C₈ alkyl substituted with C₆-C₁₄ aryl, wherein said C₆-C₁₄ aryl group is substituted with one or more fluorine;

R² is hydrogen;

R³ is —(CH₂)NR¹⁰R¹¹;

R⁴ is hydrogen;

R⁵ is hydrogen;

R⁶ is hydrogen or C₁-C₈ alkyl;

R⁷ is hydrogen or C₁-C₈ alkyl;

each R⁸ and R⁹, which may be the same or different, are independently selected from hydrogen and C₁-C₈ alkyl;

R¹⁰ and R¹¹, together with the nitrogen atom to which they are attached, form a C₂-C₉ heterocyclyl group, substituted with at least one R¹³ group;

R¹³ is selected from —(CR⁸R⁹)ₜ—OR^{15a}, —(CR⁸R⁹)ₜ—C(O)R^{15a}, —(CR⁸R⁹)ₜ—C(O)NR^{15a}R^{15b}, —(CR⁸R⁹)ₜ—S—R^{15a}, —(CR⁸R⁹)ₜ—S(O)—R^{15a}, —(CR⁸R⁹)ₜ—S(O)₂—R^{15a}, —(CR⁸R⁹)ₜ—(C₂-C₉ heterocyclyl), —(CR⁸R⁹)ₜ—(C₆-C₁₄ aryl) and —(CR⁸R⁹)ₜ—(C₂-C₉ heteroaryl);

each R^{15a} and R^{15b}, which may be the same or different, is independently selected from hydrogen and C₁-C₈ alkyl; and

each t is independently selected from 0, 1, 2, and 3; or

pharmaceutically acceptable salts or solvates thereof.

In still another aspect are provided compounds of formula (I), wherein:

R¹ is C₁-C₈ alkyl substituted with C₆-C₁₄ aryl, wherein said C₆-C₁₄ aryl group is substituted with one or more fluorine;

R² is hydrogen;

R³ is —(CH₂)NR¹⁰R¹¹;

R⁴ is hydrogen;

R⁵ is hydrogen;

R⁶ is hydrogen or C₁-C₈ alkyl;

R⁷ is hydrogen or C₁-C₈ alkyl;

R¹⁰ and R¹¹, together with the nitrogen atom to which they are attached, form a C₂-C₉ heterocyclyl group, substituted with at least one R¹³ group;

R¹³ is selected from —OR^{15a}, —C(O)R^{15a}, —C(O)NR^{15a}R^{15b}, —S—R^{15a}, —S(O)—R^{15a}, —S(O)₂R^{15a}, C₂-C₉ heterocyclyl, C₆-C₁₄ aryl and C₂-C₉ heteroaryl; and

each R^{15a} and R^{15b}, which may be the same or different, is independently selected from hydrogen and C₁-C₈ alkyl; or

pharmaceutically acceptable salts or solvates thereof.

Further still are afforded compounds of formula (I), wherein:

R¹ is 4-fluorobenzyl or 2,4-difluorobenzyl;

R² is hydrogen;

R³ is —(CH₂)NR¹⁰R¹¹;

R⁴ is hydrogen;

R⁵ is hydrogen;

R⁶ is hydrogen or C₁-C₈ alkyl;

R⁷ is hydrogen or C₁-C₈ alkyl;

R¹⁰ and R¹¹, together with the nitrogen atom to which they are attached, form a C₂-C₉ heterocyclyl group, substituted with at least one R¹³ group;

R¹³ is selected from —OR^{15a}, —C(O)R^{15a}, —C(O)NR^{15a}R^{15b}, —S—R^{15a}, —S(O)—R^{15a}, —S(O)₂—R^{15a}, C₂-C₉ heterocyclyl, C₆-C₁₄ aryl and C₂-C₉ heteroaryl; and

each R^{15a} and R^{15b}, which may be the same or different, is independently selected from hydrogen and C₁-C₈ alkyl; or

pharmaceutically acceptable salts or solvates thereof.

In still a further aspect are provided compounds of formula (I), wherein:

R¹ is 4-fluorobenzyl or 2,4-difluorobenzyl;

R² is hydrogen;

R³ is —(CH₂)NR¹⁰R¹¹;

R⁴ is hydrogen;

R⁵ is hydrogen;

R⁶ is hydrogen or —CH₃;

R⁷ is hydrogen;

R¹⁰ and R¹¹, together with the nitrogen atom to which they are attached, form a C₂-C₉ heterocyclyl group, substituted with at least one R¹³ group;

R¹³ is selected from —OR^{15a}, —C(O)R^{15a}, —C(O)NR^{15a}R^{15b}, —S—R^{15a}, —S(O)—R^{15a}, —S(O)R^{15a}, C₂-C₉ heterocyclyl, C₆-C₁₄ aryl and C₂-C₉ heteroaryl; and

each R^{15a} and R^{15b}, which may be the same or different, is independently selected from hydrogen and C₁-C₈ alkyl; or

pharmaceutically acceptable salts or solvates thereof.

In another aspect are provided compounds of formula (I), wherein:

R¹ is 4-fluorobenzyl or 2,4-difluorobenzyl;

R² is hydrogen;

R³ is —(CH₂)NR¹⁰R¹¹;

R⁴ is hydrogen;

R⁵ is hydrogen;

R⁶ is hydrogen or —CH₃;

R⁷ is hydrogen;

R¹⁰ and R¹¹, together with the nitrogen atom to which they are attached, form a C₂-C₉ heterocyclyl group, substituted with at least one R¹³ group; and

R¹³ is selected from —OH, —C(O)CH₃, —C(O)NH₂, —S(O)₂CH₃, C₂-C₉ heterocyclyl, C₆-C₁₄ aryl and C₂-C₉ heteroaryl; or

pharmaceutically acceptable salts or solvates thereof.

Further still are compounds of formula (I), wherein:

R¹ is 4-fluorobenzyl or 2,4-difluorobenzyl;

R² is hydrogen;

R³ is —(CH₂)NR¹⁰R¹¹;

R⁴ is hydrogen;

R⁵ is hydrogen;

R⁶ is hydrogen or —CH₃;

R⁷ is hydrogen;

R¹⁰ and R¹¹, together with the nitrogen atom to which they are attached, form a C₂-C₉ heterocyclyl group, substituted with at least one R¹³ group; and

R¹³ is selected from —OH, —C(O)CH₃, —C(O)NH₂, —S(O)₂CH₃; or

pharmaceutically acceptable salts or solvates thereof.

Further still are provided compounds of formula (I), wherein:

R¹ is 4-fluorobenzyl or 2,4-difluorobenzyl;

R² is hydrogen;

R³ is —(CH₂)NR¹⁰R¹¹;

R⁴ is hydrogen;

R⁵ is hydrogen;

R⁶ is hydrogen or —CH₃;

R⁷ is hydrogen; and

R¹⁰ and R¹¹, together with the nitrogen atom to which they are attached, form a C₂-C₉ heterocyclyl group substituted with —C(O)NH₂; or

pharmaceutically acceptable salts or solvates thereof.

In still a further aspect are provided compounds of formula (I), wherein:

R¹ is hydrogen, C₁-C₈ alkyl, C₂-C₈ alkenyl, or C₁-C₈ heteroalkyl, wherein said C₁-C₈ alkyl, C₂-C₈ alkenyl, or C₁-C₈ heteroalkyl groups may be optionally substituted with one or more substituent independently selected from:
halo, —OR^{15a}, N(R^{15a}R^{15b}), —C(O)N(R^{15a}R^{15b}), —NR^{15a}C(O)N(R^{15a}R^{15b}), —NR^{15a}C(O)R^{15a}, —NR^{15a}C(NR^{15a})N(R^{15a}R^{15b}), —SR^{15a}, —S(O)R^{15a}, —S(O)₂R^{15a}, —S(O)₂N(R^{15a}R^{15b}), C₁-C₈ alkyl, C₆-C₁₄ aryl, C₃-C₈ cycloalkyl, and C₂-C₉ heteroaryl, wherein said C₁-C₈ alkyl, C₆-C₁₄ aryl, C₃-C₈ cycloalkyl, and C₂-C₉ heteroaryl groups are optionally substituted with one or more substituent independently selected from halo, —C(R^{15a}R^{15b}R^{15c}), —OH, and C₁-C₈ alkoxy;

R² is hydrogen;

R³ is —(CR⁸R⁹)ₜN(R^{15a}R¹⁶);

R⁴ is hydrogen, halo, C₁-C₈ alkyl, —OR^{15a}, —NR^{15a}R^{15b}, C₁-C₈ heteroalkyl, C₂-C₈ alkenyl, or C₂-C₈ alkynyl, wherein said C₂-C₈ alkenyl or C₂-C₈ alkynyl are optionally substituted with one or more R¹² group;

R⁵ is hydrogen;

R⁶ is hydrogen, C₁-C₈ alkyl, C₁-C₈ heteroalkyl, or C₂-C₈ alkenyl, wherein said C₁-C₈ alkyl and C₂-C₈ alkenyl groups are optionally substituted with one or more C₆-C₁₄ aryl or —OR^{15a} group;

R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with one or more C₃-C₈ cycloalkyl or C₆-C₁₄ aryl group;

each R⁸ and R⁹, which may be the same or different, are independently selected from hydrogen and C₁-C₈ alkyl;

each R¹² is independently selected from —OR^{15a}, halo, C₆-C₁₄ aryl, C₂-C₉ heteroaryl, C₁-C₈ heteroalkyl, C₃-C₈ cycloalkyl, C₂-C₉ heterocyclyl, and —C(R^{15a}R^{15b}R^{15c});

each R^{15a}, R^{15b}, and R^{15c}, which may be the same or different, is independently selected from hydrogen and C₁-C₈ alkyl;

R¹⁶ is —(CH₂)ₘ—(C₂-C₉ heterocyclyl) or —(CH₂)ₘ—(C₃-C₈ cycloalkyl), wherein said C₂-C₉ heterocyclyl and C₃-C₈ cycloalkyl groups are substituted with one or more groups selected from C₃-C₈ cycloalkyl and —(CR⁸R⁹)ₜ—OR^{15a};

each m is independently selected from 0, 1, and 2; and

each t is independently selected from 0, 1, 2, and 3; or

pharmaceutically acceptable salts or solvates thereof.

In yet another aspect are provided compounds of formula (I), wherein:

R¹ is hydrogen, C₁-C₈ alkyl, C₂-C₈ alkenyl, or C₁-C₈ heteroalkyl, wherein said C₁-C₈ alkyl, C₂-C₃ alkenyl, or C₁-C₈ heteroalkyl groups may be optionally substituted with one or more substituent independently selected from:
halo, —OR^{15a}, —N(R^{15a}R^{15b}), —C(O)N(R^{15a}R^{15b}), —NR^{15a}C(O)N(R^{15a}R^{15b}), —NR^{15a}C(O)R^{15a}, —NR^{15a}C(NR^{15a})N(R^{15a}R^{15b}), —SR^{15a}, —S(O)R^{15a}, —S(O)₂R^{15a}, —S(O)₂N(R^{15a}R^{15b}), C₁-C₈ alkyl, C₆-C₁₄ aryl, C₃-C₈ cycloalkyl, and C₂-C₉ heteroaryl, wherein said C₁-C₈ alkyl, C₆-C₁₄ aryl, C₃-C₈ cycloalkyl, and C₂-C₉ heteroaryl groups are optionally substituted with one or more substituent independently selected from halo, —C(R^{15a}R^{15b}R^{15c}), —OH, and C₁-C₈ alkoxy;

R² is hydrogen;

R³ is —(CH₂)N(R^{15a}R¹⁶);

R⁴ is hydrogen, halo, C₁-C₈ alkyl, —OR^{15a}, —NR^{15a}R^{15b}, C₁-C₈ heteroalkyl, C₂-C₈ alkenyl, or C₂-C₈ alkynyl, wherein said C₂-C₈ alkenyl or C₂-C₈ alkynyl are optionally substituted with one or more R¹² group;

R⁵ is hydrogen;

R⁶ is hydrogen, C₁-C₈ alkyl, C₁-C₈ heteroalkyl, or C₂-C₈ alkenyl, wherein said C₁-C₈ alkyl and C₂-C₈ alkenyl groups are optionally substituted with one or more C₆-C₁₄ aryl or —OR^{15a} group;

R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with one or more C₈-C₈ cycloalkyl or C₆-C₁₄ aryl group;

each R⁸ and R⁹, which may be the same or different, are independently selected from hydrogen and C₁-C₈ alkyl;

each R¹² is independently selected from —OR^{15a}, halo, C₆-C₁₄ aryl, C₂-C₉ heteroaryl, C₁-C₈ heteroalkyl, C₃-C₈ cycloalkyl, C₂-C₉ heterocyclyl, and —C(R^{15a}R^{15b}R^{15c});

each R^{15a}, R^{15b}, and R^{15c}, which may be the same or different, is independently selected from hydrogen and C₁-C₈ alkyl;

R¹⁶ is —(CH₂)ₘ—(C₂-C₉ heterocyclyl) or —(CH₂)ₘ—(C₃-C₈ cycloalkyl), wherein said C₂-C₉ heterocyclyl and C₃-C₈ cycloalkyl groups are substituted with one or more groups selected from C₃-C₈ cycloalkyl and —(CR⁸R⁹)ₜ—OR^{15a};

each m is independently selected from 0, 1, and 2; and

each t is independently selected from 0, 1, 2, and 3; or

pharmaceutically acceptable salts or solvates thereof.

In still a further aspect are afforded compounds of formula (I), wherein:

R¹ is C₁-C₈ alkyl substituted with C₆-C₁₄ aryl wherein said C₆-C₁₄ aryl is optionally substituted with one or more substituent independently selected from halo, —C(R^{15a}R^{15b}R^{15c}), —OH, and C₁-C₈ alkoxy;

R² is hydrogen;

R³ is —(CH₂)N(R^{15a}R¹⁶);

R⁴ is hydrogen;

R⁵ is hydrogen;

R⁶ is hydrogen or C₁-C₈ alkyl;

R⁷ is hydrogen or C₁-C₈ alkyl;

each R^{15a}, R^{15b}, and R^{15c}, which may be the same or different, is independently selected from hydrogen and C₁-C₈ alkyl;

R¹⁶ is —(CH₂)ₘ—(C₂-C₉ heterocyclyl) or —(CH₂)ₘ—(C₃-C₈ cycloalkyl), wherein said C₂-C₉ heterocyclyl and C₃-C₈ cycloalkyl groups are substituted with one or more groups selected from C₃-C₈ cycloalkyl and —(CH₂)ₜ—OR^{15a};

each m is independently selected from 0, 1, and 2; and

each t is independently selected from 0, 1, 2, and 3; or

pharmaceutically acceptable salts or solvates thereof.

Also included herein are compounds of formula (I), wherein:

R¹ is C₁-C₈ alkyl substituted with C₆-C₁₄ aryl wherein said C₆-C₁₄ aryl is optionally substituted with one or more halo;

R² is hydrogen;

R³ is —(CH₂)N(R^{15a}R¹⁶);

R⁴ is hydrogen;

R⁵ is hydrogen;

R⁶ is hydrogen or C₁-C₈ alkyl;

R⁷ is hydrogen or C₁-C₈ alkyl;

each R^{15a}, which may be the same or different, is independently selected from hydrogen and C₁-C₈ alkyl;

R¹⁶ is —(CH₂)ₘ—(C₂-C₉ heterocyclyl) or —(CH₂)ₘ—(C₃-C₈ cycloalkyl), wherein said C₂-C₉ heterocyclyl and C₃-C₈ cycloalkyl groups are substituted with one or more groups selected from C₃-C₈ cycloalkyl and —(CH₂)ₜ—OR^{15a};

each m is independently selected from 0, 1, and 2; and

each t is independently selected from 0, 1, 2, and 3; or

pharmaceutically acceptable salts or solvates thereof.

Further still are provided compounds of formula (I), wherein:

R¹ is C₁-C₈ alkyl substituted with C₆-C₁₄ aryl wherein said C₆-C₁₄ aryl is optionally substituted with one or more fluorine;

R² is hydrogen;

R³ is —(CH₂)N(R^{15a}R¹⁶);

R⁴ is hydrogen;

R⁵ is hydrogen;

R⁶ is hydrogen or C₁-C₈ alkyl;

R⁷ is hydrogen or C₁-C₈ alkyl;

each R^{15a}, which may be the same or different, is independently selected from hydrogen and C₁-C₈ alkyl;

R¹⁶ is —(CH₂)ₘ—(C₂-C₉ heterocyclyl) or —(CH₂)ₘ—(C₃-C₈ cycloalkyl), wherein said C₂-C₉ heterocyclyl and C₃-C₈ cycloalkyl groups are substituted with one or more groups selected from C₃-C₈ cycloalkyl and —(CH₂)ₜ—OR^{15a};

each m is independently selected from 0, 1, and 2; and

each t is independently selected from 0, 1, 2, and 3; or

pharmaceutically acceptable salts or solvates thereof.

In a further aspect are afforded compounds of formula (I), wherein:

R¹ is 4-fluorobenzyl or 2,4-difluorobenzyl;

R² is hydrogen;

R³ is —(CH₂)N(R^{15a}R¹⁶);

R⁴ is hydrogen;

R⁵ is hydrogen;

R⁶ is hydrogen or C₁-C₈ alkyl;

R⁷ is hydrogen or C₁-C₈ alkyl;

each R^{15a}, which may be the same or different, is independently selected from hydrogen and C₁-C₈ alkyl;

R¹⁶ is —(CH₂)ₘ—(C₂-C₉ heterocyclyl) or —(CH₂)ₘ—(C₃-C₈ cycloalkyl), wherein said C₂-C₉ heterocyclyl and C₃-C₈ cycloalkyl groups are substituted with one or more groups selected from C₃-C₈ cycloalkyl and —(CH₂)ₜ—OR^{15a};

each m is independently selected from 0, 1, and 2; and

each t is independently selected from 0, 1, 2, and 3; or

pharmaceutically acceptable salts or solvates thereof.

In yet another aspect are provided compounds of formula (I), wherein:

R¹ is 4-fluorobenzyl or 2,4-difluorobenzyl;

R² is hydrogen;

R³ is —(CH₂)N(R^{15a}R¹⁶);

R⁴ is hydrogen;

R⁵ is hydrogen;

R⁶ is hydrogen or C₁-C₈ alkyl;

R⁷ is hydrogen or C₁-C₈ alkyl;

each R^{15a}, which may be the same or different, is independently selected from hydrogen and C₁-C₈ alkyl;

R¹⁶ is —(CH₂)ₘ—(C₂-C₉ heterocyclyl) or —(CH₂)ₘ—(C₃-C₈ cycloalkyl), wherein said C₂-C₉ heterocyclyl and C₃-C₈ cycloalkyl groups are substituted with one or more groups selected from C₃-C₈ cycloalkyl and —(CH₂)ₜ—OR^{15a};

each m is independently selected from 0, 1, and 2; and

each t is independently selected from 1 and 2; or

pharmaceutically acceptable salts or solvates thereof.

In yet another aspect are provided compounds of formula (I) selected from:
1-(2,4-difluorobenzyl)-N-hydroxy-3-{[3-(methylsulfonyl)pyrrolidin-1-yl]methyl}-1H-pyrrolo[2,3-c]pyridine-5-carboxamide;
3-{[(2S)-2-(aminocarbonyl)pyrrolidin-1-yl]methyl}-1-(2,4-difluorobenzyl)-N-hydroxy-1H-pyrrolo[2,3-c]pyridine-5-carboxamide;
1-(2,4-difluorobenzyl)-N-hydroxy-3-[(4-pyridin-2-ylpiperazin-1-yl)methyl]-1H-pyrrolo[2,3-c]pyridine-5-carboxamide;
1-(2,4-difluorobenzyl)-3-(3,4-dihydroisoquinolin-2(1H)-ylmethyl)-N-hydroxy-1H-pyrrolo[2,3-c]pyridine-5-carboxamide;
3-{[4-(aminocarbonyl)piperidin-1-yl]methyl}-1-(2,4-difluorobenzyl)-N-hydroxy-1H-pyrrolo[2,3-c]pyridine-5-carboxamide;
1-(2,4-difluorobenzyl)-N-hydroxy-3-[(3-hydroxypyrrolidin-1-yl)methyl]-1H-pyrrolo[2,3-c]pyridine-5-carboxamide;
3-[(4-acetylpiperazin-1-yl)methyl]-1-(2,4-difluorobenzyl)-N-hydroxy-1H-pyrrolo[2,3-c]pyridine-5-carboxamide;
1-(2,4-difluorobenzyl)-N-hydroxy-3-[(4-hydroxypiperidin-1-yl)methyl]-1H-pyrrolo[2,3-c]pyridine-5-carboxamide;
3-{[3-(aminocarbonyl)piperidin-1-yl]methyl}-1-(4-fluorobenzyl)-N-hydroxy-1H-pyrrolo[2,3-c]pyridine-5-carboxamide;
3-{[(2S)-2-(aminocarbonyl)pyrrolidin-1-yl]methyl}-1-(4-fluorobenzyl)-N-hydroxy-N-methyl-1H-pyrrolo[2,3-c]pyridine-5-carboxamide;
3-{[(2S)-2-(aminocarbonyl)pyrrolidin-1-yl]methyl}-1-(4-fluorobenzyl)-N-hydroxy-1H-pyrrolo[2,3-c]pyridine-5-carboxamide;
1-(4-fluorobenzyl)-N,4-dihydroxy-1H-pyrrolo[2,3-c]pyridine-5-carboxamide;
1-(4-fluorobenzyl)-N-hydroxy-4-methoxy-N-methyl-1H-pyrrolo[2,3-c]pyridine-5-carboxamide;
3-{[(2S)-2-(aminocarbonyl)pyrrolidin-1-yl]methyl}-1-(2,4-difluorobenzyl)-N-hydroxy-N-methyl-1H-pyrrolo[2,3-c]pyridine-5-carboxamide;
1-(4-fluorobenzyl)-N-hydroxy-3-{[3-(methylsulfonyl)pyrrolidin-1-yl]methyl}-1H-pyrrolo[2,3-c]pyridine-5-carboxamide;
1-(4-fluorobenzyl)-N-hydroxy-3-[(4-hydroxypiperidin-1-yl)methyl]-1H-pyrrolo[2,3-c]pyridine-5-carboxamide;
1-(4-fluorobenzyl)-N-hydroxy-3-[(4-hydroxypiperidin-1-yl)methyl]-N-methyl-1H-pyrrolo[2,3-c]pyridine-5-carboxamide;
1-(4-fluorobenzyl)-N-hydroxy-3-[(3-hydroxypyrrolidin-1-yl)methyl]-N-methyl-1H-pyrrolo[2,3-c]pyridine-5-carboxamide;
1-(4-fluorobenzyl)-N-hydroxy-N-methyl-3-{[3-(methylsulfonyl)pyrrolidin-1-yl]methyl}-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-{[(7R,8aS)-7-hydroxyhexahydropyrrolo[1,2-a]pyrazin-2(1H)-yl]methyl}-1H-pyrrolo[2,3-c]pyridine-5-carboxamide;
3-{[[2-(1-cyclopropyl-5-oxopyrrolidin-2-yl)ethyl](methyl)amino]methyl}-1-(2,4-difluorobenzyl)-N-hydroxy-1H-pyrrolo[2,3-c]pyridine-5-carboxamide;
1-(2,4-difluorobenzyl)-N-hydroxy-3-({[1-(hydroxymethyl)cyclopentyl]amino}methyl)-1H-pyrrolo[2,3-c]pyridine-5-carboxamide;
1-(2,4-difluorobenzyl)-N-hydroxy-3-({[1-(hydroxymethyl)cyclopentyl]amino}methyl)-N-methyl-1H-pyrrolo[2,3-c]pyridine-5-carboxamide;
1-(2,4-difluorobenzyl)-3-({[1-(hydroxymethyl)cyclopentyl]amino}methyl)-N-methoxy-1H-pyrrolo[2,3-c]pyridine-5-carboxamide;
3-{[[2-(1-cyclopropyl-5-oxopyrrolidin-2-yl)ethyl](methyl)amino]methyl}-1-(2,4-difluorobenzyl)-N-hydroxy-N-methyl-1H-pyrrolo[2,3-c]pyridine-5-carboxamide;
3-{[[(2-(1-cyclopropyl-5-oxopyrrolidin-2-yl)ethyl](methyl)amino]methyl}-1-(2,4-difluorobenzyl)-N-methoxy-1H -pyrrolo[2,3-c]pyridine-5-carboxamide;
3-{[3-(aminocarbonyl)piperidin-1-yl]methyl}-1-(4-fluorobenzyl)-N-hydroxy-N-methyl-1H-pyrrolo[2,3-c]pyridine-5-carboxamide;
1-(2,4-difluorobenzyl)-3-({4-hydroxy-4-[(2-oxopyrrolidin-1-yl)methyl]piperidin-1-yl}methyl)-N-methoxy-1H-pyrrolo[2,3-c]pyridine-5-carboxamide;
1-(2,4-difluorobenzyl)-N-ethyl-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-propyl-1H-pyrrolo[2,3-c]pyridine-5-carboxamide;
N-benzyl-1-(2,4-difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-methyl-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-(3-hydroxypropyl)-1H-pyrrolo[2,3-c]pyridine-5-carboxamide;
1-(2,4-difluorobenzyl)-N-ethoxy-3-({4-hydroxy-4-[(2-oxopyrrolidin-1-yl)methyl]piperidin-1-yl}methyl)-1H -pyrrolo[2,3-c]pyridine-5-carboxamide;
N-(benzyloxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide;
N-(cyclopropylmethoxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-3-({4-hydroxy-4-[(2-oxopyrrolidin-1-yl)methyl]piperidin-1-yl}methyl)-N-phenoxy-1H-pyrrolo[2,3-c]pyridine-5-carboxamide; and
1-(4-fluorobenzyl)-4-hydroxy-N-methoxy-1H-pyrrolo[2,3-c]pyridine-5-carboxamide; or
   a pharmaceutically acceptable salt or solvate thereof.

Further provided herein are compounds of formula (I) selected from:
1-(2,4-difluorobenzyl)-N-hydroxy-3-{[3-(methylsulfonyl)pyrrolidin-1-yl]methyl}-1H-pyrrolo[2,3-c]pyridine-5-carboxamide;
3-{[(2S)-2-(aminocarbonyl)pyrrolidin-1-yl]methyl}-1-(2,4-difluorobenzyl)-N-hydroxy-1H-pyrrolo[2,3-c]pyridine-5-carboxamide;
1-(2,4-difluorobenzyl)-N-hydroxy-3-[(4-pyridin-2-ylpiperazin-1-yl)methyl]-1H-pyrrolo[2,3-c]pyridine-5-carboxamide;
1-(2,4-difluorobenzyl)-3-(3,4-dihydroisoquinolin-2(1H)-ylmethyl)-N-hydroxy-1H-pyrrolo[2,3-c]pyridine-5-carboxamide;
3-{[4-(aminocarbonyl)piperidin-1-yl]methyl}-1-(2,4-difluorobenzyl)-N-hydroxy-1H-pyrrolo[2,3-c]pyridine-5-carboxamide;
1-(2,4-difluorobenzyl)-N-hydroxy-3-[(3-hydroxypyrrolidin-1-yl)methyl]-1H-pyrrolo[2,3-c]pyridine-5-carboxamide;
3-[(4-acetylpiperazin-1-yl)methyl]-1-(2,4-difluorobenzyl)-N-hydroxy-1H-pyrrolo[2,3-c]pyridine-5-carboxamide;
1-(2,4-difluorobenzyl)-N-hydroxy-3-[(4-hydroxypiperidin-1-yl)methyl]-1H-pyrrolo[2,3-c]pyridine-5-carboxamide;
3-{[3-(aminocarbonyl)piperidin-1-yl]methyl}-1-(4-fluorobenzyl)-N-hydroxy-1H-pyrrolo[2,3-c]pyridine-5-carboxamide;
3-{[(2S)-2-(aminocarbonyl)pyrrolidin-1-yl]methyl}-1-(4-fluorobenzyl)-N-hydroxy-N-methyl-1H-pyrrolo[2,3-c]pyridine-5-carboxamide;
3-{[(2S)-2-(aminocarbonyl)pyrrolidin-1-yl]methyl}-1-(4-fluorobenzyl)-N-hydroxy-1H-pyrrolo[2,3-c]pyridine-5-carboxamide;
3-{[(2S)-2-(aminocarbonyl)pyrrolidin-1-yl]methyl}-1-(2,4-difluorobenzyl)-N-hydroxy-N-methyl-1H-pyrrolo[2,3-c]pyridine-5-carboxamide;
1-(4-fluorobenzyl)-N-hydroxy-3-{[3-(methylsulfonyl)pyrrolidin-1-yl]methyl}-1H-pyrrolo[2,3-c]pyridine-5-carboxamide;
1-(4-fluorobenzyl)-N-hydroxy-3-[(4-hydroxypiperidin-1-yl)methyl]-1H-pyrrolo[2,3-c]pyridine-5-carboxamide;
1-(4-fluorobenzyl)-N-hydroxy-3-[(4-hydroxypiperidin-1-yl)methyl]-N-methyl-1H-pyrrolo[2,3-c]pyridine-5-carboxamide;
1-(4-fluorobenzyl)-N-hydroxy-3-[(3-hydroxypyrrolidin-1-yl)methyl]-N-methyl-1H-pyrrolo[2,3-c]pyridine-5-carboxamide;
1-(4-fluorobenzyl)-N-hydroxy-N-methyl-3-{[3-(methylsulfonyl)pyrrolidin-1-yl]methyl}-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-{[(7R,8aS)-7-hydroxyhexahydropyrrolo[1,2-a]pyrazin-2(1H)-yl]methyl}-1H-pyrrolo[2,3-c]pyridine-5-carboxamide;
3-{[3-(aminocarbonyl)piperidin-1-yl]methyl}-1-(4-fluorobenzyl)-N-hydroxy-N-methyl-1H-pyrrolo[2,3-c]pyridine-5-carboxamide;
1-(2,4-difluorobenzyl)-3-({4-hydroxy-4-[(2-oxopyrrolidin-1-yl)methyl]piperidin-1-yl}methyl)-N-methoxy-1H-pyrrolo[2,3-c]pyridine-5-carboxamide;
1-(2,4-difluorobenzyl)-N-ethyl-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-propyl-1H-pyrrolo[2,3-c]pyridine-5-carboxamide;
N-benzyl-1-(2,4-difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-methyl-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-(3-hydroxypropyl)-1H-pyrrolo[2,3-c]pyridine-5-carboxamide;
1-(2,4-difluorobenzyl)-N-ethoxy-3-({4-hydroxy-4-[(2-oxopyrrolidin-1-yl)methyl]piperidin-1-yl}methyl)-1H -pyrrolo[2,3-c]pyridine-5-carboxamide;
N-(benzyloxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide;
N-(cyclopropylmethoxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide; and
1-(2,4-difluorobenzyl)-3-({4-hydroxy-4-[(2-oxopyrrolidin-1-yl)methyl]piperidin-1-yl}methyl)-N-phenoxy-1H-pyrrolo[2,3-c]pyridine-5-carboxamide; or
   a pharmaceutically acceptable salt or solvate thereof.

In still a further aspect are provided compounds of formula (I) selected from:
3-{[[2-(1-cyclopropyl-5-oxopyrrolidin-2-yl)ethyl](methyl)amino]methyl}-1-(2,4-difluorobenzyl)-N-hydroxy-1H-pyrrolo[2,3-c]pyridine-5-carboxamide;
1-(2,4-difluorobenzyl)-N-hydroxy-3-({[1-(hydroxymethyl)cyclopentyl]amino}methyl)-1H-pyrrolo[2,3-c]pyridine-5-carboxamide;
1-(2,4-difluorobenzyl)-N-hydroxy-3-({[1-(hydroxymethyl)cyclopentyl]amino}methyl)-N-methyl-1H-pyrrolo[2,3-c]pyridine-5-carboxamide;
3-{[(2S)-2-(aminocarbonyl)pyrrolidin-1-yl]methyl}-1-(4-fluorobenzyl)-N-hydroxy-N-methyl-1H-pyrrolo[2,3-c]pyridine-5-carboxamide;
1-(2,4-difluorobenzyl)-3-({[1-(hydroxymethyl)cyclopentyl]amino}methyl)-N-methoxy-1H-pyrrolo[2,3-c]pyridine-5-carboxamide;
3-{[[2-(1-cyclopropyl-5-oxopyrrolidin-2-yl)ethyl](methyl)amino]methyl}-1-(2,4-difluorobenzyl)-N-hydroxy-N-methyl-1H-pyrrolo[2,3-c]pyridine-5-carboxamide; and
3-{[[2-(1-cyclopropyl-5-oxopyrrolidin-2-yl)ethyl](methyl)amino]methyl}-1-(2,4-difluorobenzyl)-N-methoxy-1H -pyrrolo[2,3-c]pyridine-5-carboxamide; or
   a pharmaceutically acceptable salt or solvate thereof.

In a further aspect are provided pharmaceutical compositions, comprising a therapeutically effective amount of at least one of any of the compounds herein, or a pharmaceutically acceptable salt or solvate thereof, and a pharmaceutically acceptable carrier or diluent.

Further provided are methods of inhibiting HIV replication in a mammal, comprising administering to said mammal an HIV-inhibiting amount of at least one of any of the compounds herein, or a pharmaceutically acceptable salt or solvate thereof.

Also afforded herein are methods of inhibiting HIV replication in a cell, comprising contacting said cell with an HIV-inhibiting amount of at least one of any of the compounds herein, or a pharmaceutically acceptable salt or solvate thereof.

Still further are provided methods of inhibiting HIV integrase enzyme activity, comprising contacting said integrase enzyme with a HIV integrase-inhibiting amount of at least one of any of the compounds herein, or a pharmaceutically acceptable salt or solvate thereof.

In yet another aspect of the present invention are afforded methods of treating acquired immune deficiency syndrome in a mammal, comprising administering to said mammal a therapeutically effective amount of at least one of any of the compounds herein, or a pharmaceutically acceptable salt or solvate thereof.

Further provided are methods of inhibiting HIV replication in a mammal, wherein said HIV is resistant to at least one HIV protease inhibitor, said method comprising administering to said mammal a therapeutically effective amount of at least one of any of the compounds herein, or a pharmaceutically acceptable salt or solvate thereof.

Also afforded herein are methods of inhibiting HIV replication in a mammal, wherein said HIV is resistant to at least one HIV reverse transcriptase inhibitor, said methods comprising administering to said mammal a therapeutically effective amount of at least one of any of the compounds herein, or a pharmaceutically acceptable salt or solvate thereof.

Further provided herein are methods of inhibiting HIV replication in mammal, comprising administering to said mammal a therapeutically effective amount of at least one of any of the compounds herein, or a pharmaceutically acceptable salt or solvate thereof, and at least one other anti-HIV agent.

Also provided herein are methods of reducing HIV viral load in a mammal infected with HIV, comprising administering to said mammal a therapeutically effective amount of at least one of any of the compounds herein, or a pharmaceutically acceptable salt or solvate thereof.

Further provided are uses of compounds herein, or a pharmaceutically acceptable salt or solvate thereof, in the manufacture of a medicament for the treatment of acquired immune deficiency syndrome (AIDS) or AIDS-related complex in a mammal.

Also provided herein are methods for treating HIV and HCV infections in a co-infected mammal, comprising administering at least one compound according to formula (I), or a pharmaceutically acceptable salt or solvate thereof, in combination with at least one anti-HCV agent.

As used herein, the terms “comprising” and “including” are used in their open, non-limiting sense.

As used herein, the term “HIV” means Human Immunodeficiency Virus. The term “HIV integrase,” as used herein, means the Human Immunodeficiency Virus integrase enzyme.

The term “C₁-C₈ alkyl”, as used herein, means saturated monovalent hydrocarbon radicals having straight or branched moieties and containing from 1 to 8 carbon atoms. Examples of such groups include, but are not limited to, methyl, ethyl, propyl, iso-propyl, n-butyl, iso-butyl, and tert-butyl.

The term “C₁-C₈ heteroalkyl” refers to a straight- or branched-chain alkyl group having a total of from 2 to 12 atoms in the chain, including from 1 to 8 carbon atoms, and one or more atoms of which is a heteroatom selected from S, O, and N, with the proviso that said chain may not contain two adjacent O atoms or two adjacent S atoms, and with the proviso that no heteroatom may be attached directly to the five-membered ring at the R³ position in the compounds of formula (I), directly to the five-membered ring at the R¹⁴ position in the compounds of formula (II), or directly to the five-membered ring in any other compounds of the present invention. The S atoms in said chains may be optionally oxidized with one or two oxygen atoms, to afford sulfoxides and sulfones, respectively. Furthermore, the C₁-C₈ heteroalkyl groups in the compounds of the present invention can contain an oxo group at any carbon or heteroatom that will result in a stable compound, with the proviso that no carbonyl (C═O) group may be attached directly to the 1H-pyrrolo[2,3-c]pyridine core at the R³ position in the compounds of formula (I), directly to the 1H-pyrazolo[3,4-c]pyridine core at the R¹⁴ position in the compounds of formula (II), or directly to the five-membered ring of the core in any other compound of the present invention. Exemplary C₁-C₈ heteroalkyl groups include, but are not limited to, alcohols, alkyl ethers, primary, secondary, and tertiary alkyl amines, amides, ketones, esters, alkyl sulfides, and alkyl sulfones.

The term “C₂-C₈ alkenyl”, as used herein, means an alkyl moiety comprising 2 to 8 carbons having at least one carbon-carbon double bond. The carbon-carbon double bond in such a group may be anywhere along the 2 to 8 carbon chain that will result in a stable compound. Such groups include both the E and Z isomers of said alkenyl moiety. Examples of such groups include, but are not limited to, ethenyl, propenyl, butenyl, allyl, and pentenyl. The term “allyl,” as used herein, means a —CH₂CH═CH₂ group.

As used herein, the term “C₂-C₈ alkynyl” means an alkyl moiety comprising from 2 to 8 carbon atoms and having at least one carbon-carbon triple bond. The carbon-carbon triple bond in such a group may be anywhere along the 2 to 8 carbon chain that will result in a stable compound. Examples of such groups include, but are not limited to, ethyne, propyne, 1-butyne, 2-butyne, 1-pentyne, 2-pentyne, 1-hexyne, 2-hexyne, and 3-hexyne.

The term “C₃-C₈ cycloalkyl group” means a saturated, monocyclic, fused, or spiro, polycyclic ring structure having a total of from 3 to 8 carbon ring atoms. Examples of such groups include, but are not limited to, cyclopropyl, cyclobutyl, cyclopentyl, cyclopentenyl, cyclohexyl, cycloheptyl, and adamantyl.

The term “C₆-C₁₄ aryl”, as used herein, means a group derived from an aromatic hydrocarbon containing from 6 to 14 carbon atoms. Examples of such groups include, but are not limited to, phenyl or naphthyl. The terms “Ph” and “phenyl,” as used herein, mean a —C₆H₅ group.

The term “benzyl,” as used herein, means a —CH₂C₆H₅ group.

The term “C₂-C₉ heteroaryl,” as used herein, means an aromatic heterocyclic group having a total of from 5 to 10 atoms in its ring, and containing from 2 to 9 carbon atoms and from one to four heteroatoms each independently selected from O, S and N, and with the proviso that the ring of said group does not contain two adjacent O atoms or two adjacent S atoms. The heterocyclic groups include benzo-fused ring systems. Examples of aromatic heterocyclic groups include, but are not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The C₂-C₉ heteroaryl groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “C₂-C₉ heterocyclyl,” as used herein, means a non-aromatic, monocyclic, bicyclic, tricyclic, tetracyclic, or spirocyclic group having a total of from 3 to 10 atoms in its ring system, and containing from 2 to 9 carbon atoms and from one to four heteroatoms each independently selected from O, S and N, and with the proviso that the ring of said group does not contain two adjacent O atoms or two adjacent S atoms. Furthermore, such C₂-C₉ cycloheteroalkyl groups may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a C₂-C₉ cycloheteroalkyl group contains a sulfur atom, said sulfur atom may be oxidized with one or two oxygen atoms to afford either a sulfoxide or sulfone. An example of a 4 membered heterocyclic group is azetidinyl (derived from azetidine). An example of a 5 membered heterocyclic group is thiazolyl and an example of a 10 membered heterocyclic group is quinolinyl. Further examples of such C₂-C₉ cycloheteroalkyl groups include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl.

The term “C₁-C₈ alkoxy”, as used herein, means an O-alkyl group wherein said alkyl group contains from 1 to 8 carbon atoms and is straight, branched, or cyclic. Examples of such groups include, but are not limited to, methoxy, ethoxy, n-propyloxy, iso-propyloxy, n-butoxy, iso-butoxy, tert-butoxy, cyclopentyloxy, and cyclohexyloxy.

The terms “halogen” and “halo,” as used herein, mean fluorine, chlorine, bromine or iodine.

The term “substituted,” means that the specified group or moiety bears one or more substituents. The term “unsubstituted,” means that the specified group bears no substituents. The term “optionally substituted” means that the specified group is unsubstituted or substituted by one or more substituents. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one to which the remainder of the compound of the present invention is bonded, minus an additional substituent, to leave 4). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C6 aryl has 3 carbon atoms remaining that are unsubstituted. Those three unsubstituted carbon atoms are each bound to one hydrogen atom to fill their valencies.

The term “solvate,” as used herein, means a pharmaceutically acceptable solvate form of a compound of the present invention that retains the biological effectiveness of such compound. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof. It is specifically contemplated that in the present invention one solvent molecule can be associated with one molecule of the compounds of the present invention, such as a hydrate. Furthermore, it is specifically contemplated that in the present invention, more than one solvent molecule may be associated with one molecule of the compounds of the present invention, such as a dihydrate. Additionally, it is specifically contemplated that in the present invention less than one solvent molecule may be associated with one molecule of the compounds of the present invention, such as a hemihydrate. Furthermore, solvates of the present invention are contemplated as solvates of compounds of the present invention that retain the biological effectiveness of the non-hydrate form of the compounds.

The term “pharmaceutically acceptable salt,” as used herein, means a salt of a compound of the present invention that retains the biological effectiveness of the free acids and bases of the specified derivative and that is not biologically or otherwise undesirable.

The term “pharmaceutically acceptable formulation,” as used herein, means a combination of a compound of the invention, or a pharmaceutically acceptable salt or solvate thereof, and a carrier, diluent, and/or excipients that are compatible with a compound of the present invention, and is not deleterious to the recipient thereof. Pharmaceutical formulations can be prepared by procedures known to those of ordinary skill in the art. For example, the compounds of the present invention can be formulated with common excipients, diluents, or carriers, and formed into tablets, capsules, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as povidone, sodium starch glycolate, sodium carboxymethylcellulose, agar, calcium carbonate, and sodium bicarbonate; agents for retarding dissollution such as paraffin; resorption accelerators such as quaternary ammonium compounds; surfacelactive agents such as cetyl alcohol, glycerol monostearate; adsorptive carriers such as kaolin and bentonite; and lubricants such as talc, calcium and magnesium stearate and solid polyethylene glycols. Final pharmaceutical forms may be pills, tablets, powders, lozenges, saches, cachets, or sterile packaged powders, and the like, depending on the type of excipient used. Additionally, it is specifically contemplated that pharmaceutically acceptable formulations of the present invention can contain more than one active ingredient. For example, such formulations may contain more than one compound according to the present invention. Alternatively, such formulations may contain one or more compounds of the present invention and one or more additional anti-HIV agents.

The term “inhibiting HIV replication” means inhibiting human immunodeficiency virus (HIV) replication in a cell. Such a cell may be present in vitro, or it may be present in vivo, such as in a mammal, such as a human. Such inhibition may be accomplished by administering a compound of the present invention, or a pharmaceutically acceptable salt or solvate thereof, to the cell, such as in a mammal, in an HIV-inhibiting amount. The quantification of inhibition of HIV replication in a cell, such as in a mammal, can be measured using methods known to those of ordinary skill in the art. For example, an amount of a compound of the invention may be administered to a mammal, either alone or as part of a pharmaceutically acceptable formulation. Blood samples may then be withdrawn from the mammal and the amount of HIV virus in the sample may be quantified using methods known to those of ordinary skill in the art. A reduction in the amount of HIV virus in the sample compared to the amount found in the blood before administration of a compound of the invention would represent inhibition of the replication of HIV virus in the mammal. The administration of a compound of the invention to the cell, such as in a mammal, may be in the form of single dose or a series of doses. In the case of more than one dose, the doses may be administered in one day or they may be administered over more than one day.

An “HIV-inhibiting agent” means a compound of the present invention or a pharmaceutically acceptable salt or solvate thereof.

The term “anti-HIV agent,” as used herein, means a compound or combination of compounds capable of inhibiting the replication of HIV in a cell, such as a cell in a mammal. Such compounds may inhibit the replication of HIV through any mechanism known to those of ordinary skill in the art.

The terms “human immunodeficiency virus-inhibiting amount” and “HIV-inhibiting amount,” as used herein, refer to the amount of a compound of the present invention, or a pharmaceutically acceptable salt of solvate thereof, required to inhibit replication of the human immunodeficiency virus (HIV) in vivo, such as in a mammal, or in vitro. The amount of such compounds required to cause such inhibition can be determined without undue experimentation using methods described herein and those known to those of ordinary skill in the art.

The term “inhibiting HIV integrase enzyme activity,” as used herein, means decreasing the activity or functioning of the HIV integrase enzyme either in vitro or in vivo, such as in a mammal, such as a human, by contacting the enzyme with a compound of the present invention.

The term “HIV integrase enzyme-inhibiting amount,” as used herein, refers to the amount of a compound of the present invention, or a pharmaceutically acceptable salt or solvate thereof, required to decrease the activity of the HIV integrase enzyme either in vivo, such as in a mammal, or in vitro. Such inhibition may take place by the compound of the present invention binding directly to the HIV integrase enzyme. In addition, the activity of the HIV integrase enzyme may be decreased in the presence of a compound of the present invention when such direct binding between the enzyme and the compound does not take place. Furthermore, such inhibition may be competitive, non-competitive, or uncompetitive. Such inhibition may be determined using in vitro or in vivo systems, or a combination of both, using methods known to those of ordinary skill in the art.

The term “therapeutically effective amount,” as used herein, means an amount of a compound of the present invention, or a pharmaceutically acceptable salt or solvate thereof, that, when administered to a mammal in need of such treatment, is sufficient to effect treatment, as defined herein. Thus, a therapeutically effective amount of a compound of the present invention, or a pharmaceutically acceptable salt or solvate thereof, is a quantity sufficient to modulate or inhibit the activity of the HIV integrase enzyme such that a disease condition that is mediated by activity of the HIV integrase enzyme is reduced or alleviated.

The terms “treat”, “treating”, and “treatment” refer to any treatment of an HIV integrase mediated disease or condition in a mammal, particularly a human, and include: (i) preventing the disease or condition from occurring in a subject which may be predisposed to the condition, such that the treatment constitutes prophylactic treatment for the pathologic condition; (ii) modulating or inhibiting the disease or condition, i.e., arresting its development; (iii) relieving the disease or condition, i.e., causing regression of the disease or condition; or (iv) relieving and/or alleviating the disease or condition or the symptoms resulting from the disease or condition, e.g., relieving an inflammatory response without addressing the underlying disease or condition.

The terms “resistant,” “resistance,” and “resistant HIV,” as used herein, refer to HIV virus demonstrating a reduction in sensitivity to a particular drug. A mammal infected with HIV that is resistant to a particular anti-HIV agent or combination of agents usually manifests an increase in HIV viral load despite continued administration of the agent or agents. Resistance may be either genotypic, meaning that a mutation in the HIV genetic make-up has occurred, or phenotypic, meaning that resistance is discovered by successfully growing laboratory cultures of HIV virus in the presence of an anti-HIV agent or a combination of such agents.

The terms “protease inhibitor” and “HIV protease inhibitor,” as used herein, refer to compounds or combinations of compounds that interfere with the proper functioning of the HIV protease enzyme that is responsible for cleaving long strands of viral protein into the separate proteins making up the viral core.

The terms “reverse transcriptase inhibitor” and “HIV reverse transcriptase inhibitor,” as used herein, refer to compounds or combinations of compounds that interfere with the proper functioning of the HIV reverse transcriptase enzyme that is responsible for converting single-stranded HIV viral RNA into HIV viral DNA.

The terms “fusion inhibitor” and “HIV fusion inhibitor,” as used herein, refer to compounds or combinations of compounds that bind to the gp41 envelope protein on the surface of CD4 cells and thereby block the structural changes necessary for the virus to fuse with the cell.

The terms “integrase inhibitor” and “HIV integrase inhibitor,” as used herein, refer to a compound or combination of compounds that interfere with the proper functioning of the HIV integrase enzyme that is responsible for inserting the genes of HIV into the DNA of a host cell.

The term “CCR5 antagonist,” as used herein, refer to compounds or combinations of compounds that block the infection of certain cell types by HIV through the perturbation of CCR5 co-receptor activity.

The terms “viral load” and “HIV viral load,” as used herein, mean the amount of HIV in the circulating blood of a mammal, such as a human. The amount of HIV virus in the blood of mammal can be determined by measuring the quantity of HIV RNA in the blood using methods known to those of ordinary skill in the art.

The term “HCV,” as used herein, refers to Hepatitis C virus.

The term “HCV infected mammal,” as used herein, means a mammal, such as a human, that is infected with the Hepatitis C virus and is in need of treatment to prevent further progression of or a cure for HCV-related conditions or illnesses. Such treatment may take the form of administration to the mammal of a therapeutically effective amount of a combination of two or more compounds having anti-HCV activity or pharmaceutically acceptable formulations containing them.

An “HCV-inhibiting agent” or “an agent having anti-HCV activity,” means a compound capable of inhibiting replication of the Hepatitis C virus, either in vitro, such as in a cell culture, or in vivo, such as in a mammal, such as a human. It is specifically contemplated that the term “agent” is meant to include so-called “small molecules,” with molecular weights below about 500, as well as larger molecules, such as therapeutic proteins, such as interferons, with molecular weights above 500. All such compounds that are able to inhibit replication of the HCV virus, by whatever mechanism, are meant to be included within the scope of the present invention.

The term “target,” as used herein, refers to any Hepatitis C virus-specific protein or life-cycle event required for the virus to replicate in a normal fashion either in vitro, such as in cell culture, or in vivo, such as in a mammal, such as a human. Such “targets” include, but are not limited to, the HCV metalloprotease enzyme, the HCV serine protease enzyme, the HCV polymerase enzyme, the HCV helicase enzyme, the HCV NS4B protein, the HCV NS5A protein, the HCV entry event, the HCV assembly event, and the HCV egress event.

The term “HCV metalloprotease,” as used herein means a nonstructural protein (NS2/3), responsible for cis cleavage at the NS2/3 junction of the HCV polyproteins. See, for example, Whitney M., Stack, J. H., Darke, P. L., Zheng, W., Terzo, J., Inglese, J., Strulovici, B., Kuo, L. C., Pollock, B. A. “A collaborative screening program for the discovery of inhibitors of HCV NS2/3 cis-cleaving protease activity,” J Biomol Screen., 7, 149-154, 2002; and Pieroni, L., Santolini, E., Fipaldini, C., Pacini, L., Migliaccio, G., La Monica, N., “In vitro study of the NS2-3 protease of hepatitis C virus,” J Virol., 71, 6373-6380, 1997.

As used herein, the term “HCV serine protease,” is an HCV-specific protein, also called the NS3 protease, responsible for cleavage of the remaining HCV NS proteins in a host cell. See, for example, Lamarre, D., Anderson, P. C., Bailey, M., Beaulieu, P., Bolger, G., Bonneau, P., Bos, M., Cameron, D. R., Cartier, M., Cordingley, M. G., Faucher, A. M., Goudreau, N., Kawai, S. H., Kukoij, G., Lagace, L., LaPlante, S. R., Narjes, H., Poupart, M. A. Rancourt, J., Sentjens, R. E., St. George, R., Simoneau, B., Steinmann, G., Thibeault, D., Tsantrizos, Y S., Weldon, S. M., Yong, C. L., Llinas-Brunet, M., “An NS3 protease inhibitor with antiviral effects in humans infected with hepatitis C virus,” Nature, 426,186-189, 2003. Tomei, L., Failla, C., Santolini, E., De Francesco, R., La Monica, N., “NS3 is a serine protease required for processing of hepatitis C virus polyprotein,” J Virol., 67, 4017-4026, 1993. De Francesco, R., Tomei, L., Altamura, S., Summa, V., Migliaccio, G., “Approaching a new era for hepatitis C virus therapy: inhibitors of the NS3-4A serine protease and the NS5B RNA-dependent RNA polymerase,” Antiviral Res., 58, 1-16, 2003.

The term “HCV polymerase,” means an HCV-specific protein, also called the NS5B protein, and is an RNA-dependent RNA polymerase responsible for the synthesis of HCV RNA in a host cell. See, for example, Dhanak, D., Duffy, K. J., Johnston, V. K., Lin-Goerke, J., Darcy, M., Shaw, A. N., Gu, B., Silverman, C., Gates, A. T., Normemacher, M. R., Earnshaw, D. L., Casper, D. J., Kaura, A., Baker, A., Greenwood, C., Gutshall, L. L., Maley, D., DelVecchio, A., Macarron, R., Hofmann, G. A, Alnoah, Z., Cheng, H. Y., Chan, G., Khandekar, S., Keenan, R. M., Sarisky, R. T., “Identification and biological characterization of heterocyclic inhibitors of the hepatitis C virus RNA-dependent RNA polymerase,” J Biol. Chem., 277, 38322-38327, 2002. De Francesco, R., Tomei, L., Altamura, S., Summa, V. Migliaccio, G., “Approaching a new era for hepatitis C virus therapy: inhibitors of the NS3-4A serine protease and the NS5B RNA-dependent RNA polymerase,” Antiviral Res., 58, 1-16, 2003.

As used herein, the term “HCV helicase,” means an HCV-specific protein, also called the NS3 helicase domain, and is responsible for RNA unwinding during HCV RNA replication in a host cell. See, for example, Kwong A D, Kim J L, Lin C., “Structure and function of hepatitis C virus NS3 helicase,” Curr Top Microbiol Immunol., 242, 171-196, 2000. Yao, N., Weber, P C., “Helicase, a target for novel inhibitors of hepatitis C virus,” Antivir Ther., 3(S3), 93-97, 1998.

The term “HCV NS4B protein” means an HCV-specific protein, the function of which is currently unknown. Hugle, T., Fehrmann, F., Bieck, E., Kohara, M., Krausslich, H. G., Rice, C. M., Blum, H. E., Moradpour, D., “The hepatitis C virus nonstructural protein 4B is an integral endoplasmic reticulum membrane protein,” Virology, 284, 70-81, 2001.

The term “HCV NS5A protein,” means an HCV-specific protein, the function of which is currently unknown but is postulated to be related to interferon responsiveness. See, for example, PCT Publication No. WO2004014313. Tan, S. L., Katze, M. G., “How hepatitis C virus counteracts the interferon response: the jury is still out on NS5A,” Virology, 284, 1-12, 2001.

The term “HCV entry” means a series of processes that take place during HCV replication in a host cell. Steps included in HCV entry into a host cell include, but are not limited to envelope-receptor binding, membrane fusion and virus penetration into the host cell. It is specifically contemplated that the methods and compositions described herein can function by inhibiting or otherwise interrupting the normal functioning of any of these processes.

“HCV assembly refers to the process of the formation of HCV virus particles in a host cell. It is specifically contemplated that the methods and compositions described herein can function by inhibiting or otherwise interrupting the normal functioning of any of these processes.

“HCV egress” refers to the process of virus budding and release from infected host cells. It is specifically contemplated that the methods and compositions described herein can function by inhibiting or otherwise interrupting the normal functioning of any of these processes.

The term “HCV IRES,” as used herein, means an internal ribosomal entry site, required for the translation of HCV proteins. See, for example, Hanecak, R., Brown-Driver, V., Fox, M. C., Azad, R. F., Furusako, S., Nozaki, C., Ford, C., Sasmor, H., Anderson, K. P., “Antisense oligonucleotide inhibition of hepatitis C virus gene expression in transformed hepatocytes,” J Virol. 70, 5203-5212, 1996. Jubin, R., “Targeting hepatitis C virus translation: stopping HCV where it starts,” Curr Opin Investig Drugs, 4,162-167, 2003.

The term “interferon” means any of a family of glycoproteins, usually produced as a result of infection of a host cell, that exhibit virus-nonspecific but host-specific antiviral activity by inducing the transcription of cellular genes coding for anti-viral proteins that selectively inhibit the synthesis of viral RNA and proteins.

The term “ribavirin” means a compound also known as 1-beta-D-ribofuranosyl-1,2,4-triazole-3-carboxamide or 1-β-D-ribofuranosyl-1H-1,2,4-triazole-3-carboxamide, and having the Chemical Abstracts registry number [36791-04-5].

The term “ciluprevir” means a compound having the chemical name (2R,6S,12Z,13aS,14aR,16aS)-6-[[(cyclopentyloxy)carbonyl]amino]-2-[[7-methoxy-2-[2-[(1-methylethyl)amino]thiazol-4-yl]quinolin-4-yl]oxy]-5,16-dioxo-1,2,3,6,7,8,9,10,11,13a,14,15,16,16a-tetradecahydrocyclopropa[e]pyrrolo[1,2-a][1,4]diazacyclopentadecine-14a(51)-carboxylic acid and the Chemical Abstracts registry number [300832-84-2].

The term “a mammal co-infected with HCV and HIV,” as used herein, means a mammal, such as a human, that is suffering from infection with both the Hepatitis C virus and the Human Immunodeficiency virus at the same time and is in need of treatment to prevent further progression of HCV-related conditions or illnesses, or HIV-related conditions or illnesses, or conditions or illnesses related to infection with both HCV and HIV.

The terms “inhibiting Hepatitis C virus,” “inhibiting Hepatitis C virus replication,” and “anti-HCV activity,” mean inhibiting Hepatitis C virus replication either in vitro, such as in a cell culture, or in vivo, such as in a mammal, such as a human, by contacting the Hepatitis C virus with an HCV-replication inhibiting amount of an agent capable of affecting such inhibition by any mechanism, whether or not currently understood. Such inhibition may take place in vitro, such as in a cell culture, by brining the agent or combination of agents into contact with, for example, an HCV-infected cell or a purified protein derived from the HCV virus, or a derivative thereof, such as the HCV polymerase enzyme. Alternatively, such inhibition may take place in vivo, such as in a mammal, such as a human, by administering to the mammal a Hepatitis C virus-inhibiting amount of an agent according to the present invention. The amount of a particular anti-HCV agent according to the present invention that is necessary to inhibit replication of the HCV virus either in vitro or in vivo, such as in a mammal, such as a human, can be determined using methods known to those of ordinary skill in the art. For example, an amount of an agent or combination of agents according to the invention may be administered to a mammal, either alone or as part of a pharmaceutically acceptable formulation. Blood samples may then be withdrawn from the mammal and the amount of Hepatitis C virus in the sample may be quantified using methods known to those of ordinary skill in the art. A reduction in the amount of Hepatitis C virus in the sample compared to the amount found in the blood before administration of an agent or combination of agents according to the present invention would represent inhibition of the replication of Hepatitis C virus in the mammal. The administration to the mammal of an agent or combination of agents according to the present invention may be in the form of single dose or a series of doses over successive days. Furthermore, if two or more agents according to the invention are being used in combination, they may be administered as part of the same formulation or they may be administered in separate formulations. When administered in separate formulations, they may be administered at the same time or they may be administered sequentially with an appropriate amount of time in between.

The term “HCV-inhibiting amount,” as used herein, refers to an amount of a compound of the present invention that is sufficient to inhibit the replication of the Hepatitis C virus when administered to a mammal, such as a human.

The term “HCV polymerase-inhibiting amount,” as used herein, means an amount of a composition of the present invention that is sufficient to inhibit the function of the Hepatitis C virus polymerase enzyme when placed in contact with the enzyme.

The terms “co-administration” or “co-administering,” as used herein, refer to the administration of a combination of a first agent and a second agent according to the present invention. Such co-administration can be performed such that the first agent and the second agent are part of the same composition or part of the same unitary dosage form. Co-administration also includes administering a first agent and a second agent separately, but as part of the same therapeutic regimen. The two components, if administered separately, need not necessarily be administered at essentially the same time, although they can be if so desired. Thus co-administration includes, for example, administering a first agent and a second agent as separate dosages or dosage forms, but at the same time. Co-administration also includes separate administration at different times and in any order.

The term, “compound of the present invention” refers to any of the above-mentioned compounds, as well as those in the Examples that follow, and include those generically described or those described as species. The term also refers to pharmaceutically acceptable salts or solvates of these compounds.

### DETAILED DESCRIPTION

The compounds of the present invention are useful for modulating or inhibiting HIV integrase enzyme. More particularly, the compounds of the present invention are useful as modulators or inhibitors of HIV integrase activity, and thus are useful for the prevention and/or treatment of HIV mediated diseases or conditions (e.g., AIDS, and ARC), alone or in combination with other known antiviral agents.

In accordance with a convention used in the art, the symbol 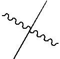
is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g., 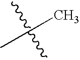
represents a methyl group, 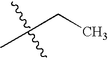
represents an ethyl group, 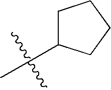
represents a cyclopentyl group, etc.

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another. The terms “racemic” or “racemic mixture,” as used herein, refer to a 1:1 mixture of enantiomers of a particular compound. The term “diastereomers”, on the other hand, refers to the relationship between a pair of stereoisomers that comprise two or more asymmetric centers and are not mirror images of one another.

The compounds of the present invention may have asymmetric carbon atoms. The carbon-carbon bonds of the compounds of the present invention may be depicted herein using a solid line 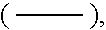
a solid wedge [symbol] or a dotted wedge [symbol] The use of a solid line to depict bonds from asymmetric carbon atoms is meant to indicate that all possible stereoisomers at that carbon atom are included. The use of either a solid or dotted wedge to depict bonds from asymmetric carbon atoms is meant to indicate that only the stereoisomer shown is meant to be included. It is possible that compounds of the invention may contain more than one asymmetric carbon atom. In those compounds, the use of a solid line to depict bonds from asymmetric carbon atoms is meant to indicate that all possible stereoisomers are meant to be included. The use of a solid line to depict bonds from one or more asymmetric carbon atoms in a compound of the invention and the use of a solid or dotted wedge to depict bonds from other asymmetric carbon atoms in the same compound is meant to indicate that a mixture of diastereomers is present.

If a derivative used in the method of the invention is a base, a desired salt may be prepared by any suitable method known to the art, including treatment of the free base with an inorganic acid, such as hydrochloric acid; hydrobromic acid; sulfuric acid; nitric acid; phosphoric acid; and the like, or with an organic acid, such as acetic acid; maleic acid; succinic acid; mandelic acid; fumaric acid; malonic acid; pyruvic acid; oxalic acid; glycolic acid; salicylic acid; pyranosidyl acid, such as glucuronic acid or galacturonic acid; alpha-hydroxy acid, such as citric acid or tartaric acid; amino acid, such as aspartic acid or glutamic acid; aromatic acid, such as benzoic acid or cinnamic acid; sulfonic acid, such as p-toluenesulfonic acid or ethanesulfonic acid; and the like.

If a derivative used in the method of the invention is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

A “solvate” is intended to mean a pharmaceutically acceptable solvate form of a specified compound that retains the biological effectiveness of such compound. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof.

A “pharmaceutically acceptable salt” is intended to mean a salt that retains the biological effectiveness of the free acids and bases of the specified derivative, containing pharmacologically acceptable anions, and is not biologically or otherwise undesirable. Examples of pharmaceutically acceptable salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phospate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds, agents and salts may exist in different crystal or polymorphic forms, all of which are intended to be within the scope of the present invention and specified formulas.

The compounds of the present invention may be formulated into pharmaceutical compositions as described below in any pharmaceutical form recognizable to the skilled artisan as being suitable. Pharmaceutical compositions of the invention comprise a therapeutically effective amount of at least one compound of the present invention and an inert, pharmaceutically acceptable carrier or diluent.

To treat or prevent diseases or conditions mediated by HIV, a pharmaceutical composition of the invention is administered in a suitable formulation prepared by combining a therapeutically effective amount (i.e., an HIV Integrase modulating, regulating, or inhibiting amount effective to achieve therapeutic efficacy) of at least one compound of the present invention (as an active ingredient) with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol®, Gelucire® or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g., parenteral or oral administration.

To obtain a stable water-soluble dose form, a pharmaceutically acceptable salt of a compound of the present invention may be dissolved in an aqueous solution of an organic or inorganic acid, such as 0.3 M solution of succinic acid or citric acid. If a soluble salt form is not available, the agent may be dissolved in a suitable cosolvent or combinations of cosolvents. Examples of suitable cosolvents include alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds of the present invention may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use.

In addition to the formulations described above, the compounds of the present invention may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD: 5 W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil, Labrasol®, Lauroglycol®, Plurol®, Peceol® Transcutol® and the like may be used. Further, the pharmaceutical composition may be incorporated into a skin patch for delivery of the drug directly onto the skin.

It will be appreciated that the actual dosages of the agents of this invention will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations of the present invention may contain a compound of the present invention, or a pharmaceutically acceptable salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg.

Additionally, the pharmaceutically acceptable formulations of the present invention may contain a compound of the present invention, or a pharmaceutically acceptable salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds of the present invention, or a pharmaceutically acceptable salt or solvate thereof, may be administered to a mammal suffering from infection with HIV, such as a human, either alone or as part of a pharmaceutically acceptable formulation, once a day, twice a day, or three times a day.

Those of ordinary skill in the art will understand that with respect to the compounds of the present invention, the particular pharmaceutical formulation, the dosage, and the number of doses given per day to a mammal requiring such treatment, are all choices within the knowledge of one of ordinary skill in the art and can be determined without undue experimentation. For example, see “Guidelines for the Use of Antiretroviral Agents in HIV-1 Infected Adults and Adolescents,” United States Department of Health and Human Services, available at http://www.aidsinfo.nih.gov/quidelines/ as of Apr. 16, 2004.

The compounds of the present invention may be administered in combination with an additional agent or agents for the treatment of a mammal, such as a human, that is suffering from an infection with the HIV virus, AIDS, AIDS-related complex (ARC), or any other disease or condition which is related to infection with the HIV virus. The agents that may be used in combination with the compounds of the present invention include, but are not limited to, those useful as HIV protease inhibitors, HIV reverse transcriptase inhibitors, non-nucleoside HIV reverse transcriptase inhibitors, inhibitors of HIV integrase, CCR5 inhibitors, HIV fusion inhibitors, compounds useful as immunomodulators, compounds that inhibit the HIV virus by an unknown mechanism, compounds useful for the treatment of herpes viruses, compounds useful as anti-infectives, and others as described below.

Compounds useful as HIV protease inhibitors that may be used in combination with the compounds of the present invention include, but are not limited to, 141 W94 (amprenavir), CGP-73547, CGP-61755, DMP-450, nelfinavir, saquinavir (invirase), TMC-126, atazanavir, palinavir, GS-3333, KNI-413, KNI-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium (Lexiva), benzenesulfonamide derivatives disclosed in WO 03053435, R-944, Ro-03-34649, VX-385, GS-224338, OPT-TL3, PL-100, SM-309515, AG-148, DG-35-VIII, DMP-850, GW-5950X, KNI-1039, L-756423, LB-71262, LP-130, RS-344, SE-063, UIC-94-003, Vb-19038, A-77003, BMS-182193, BMS-186318, SM-309515, JE-2147, GS-9005.

Compounds useful as inhibitors of the HIV reverse transcriptase enzyme that may be used in combination with the compounds of the present invention include, but are not limited to, abacavir, FTC, GS-840, lamivudine, adefovir dipivoxil, beta-fluoro-ddA, zalcitabine, didanosine, stavudine, zidovudine, tenofovir, amdoxovir, SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine, and alovudine.

Compounds useful as non-nucleoside inhibitors of the HIV reverse transcriptase enzyme that may be used in combination with the compounds of the present invention include, but are not limited to, efavirenz, HBY-097, nevirapine, TMC-120 (dapivirine), TMC-125, etravirine, delavirdine, DPC-083, DPC-961, TMC-120, capravirine, GW-678248, GW-695634, calanolide, and tricyclic pyrimidinone derivatives as disclosed in WO 03062238.

Compounds useful as CCR5 inhibitors that may be used in combination with the compounds of the present invention include, but are not limited to, TAK-779, SC-351125, SCH-D, UK-427857, PRO-140, and GW-873140 (Ono-4128, AK-602).

Compounds useful as inhibitors of HIV integrase enzyme that may be used in combination with the compounds of the present invention include, but are not limited to, GW-810781, 1,5-naphthyridine-3-carboxamide derivatives disclosed in WO 03062204, compounds disclosed in WO 03047564, compounds disclosed in WO 03049690, and 5-hydroxypyrimidine-4-carboxamide derivatives disclosed in WO 03035076.

Fusion inhibitors for the treatment of HIV that may be used in combination with the compounds of the present invention include, but are not limited to enfuvirtide (T-20), T-1249, AMD-3100, and fused tricyclic compounds disclosed in JP 2003171381.

Other compounds that are useful inhibitors of HIV that may be used in combination with the compounds of the present invention include, but are not limited to, Soluble CD4, TNX-355, PRO-542, BMS-806, tenofovir disoproxil fumarate, and compounds disclosed in JP 2003119137.

Compounds useful in the treatment or management of infection from viruses other than HIV that may be used in combination with the compounds of the present invention include, but are not limited to, acyclovir, fomivirsen, penciclovir, HPMPC, oxetanocin G, AL-721, cidofovir, cytomegalovirus immune globin, cytovene, fomivganciclovir, famciclovir, foscarnet sodium, Isis 2922, KNI-272, valacyclovir, virazole ribavirin, valganciclovir, ME-609, PCL-016 Compounds that act as immunomodulators and may be used in combination with the compounds of the present invention include, but are not limited to, AD-439, AD-519, Alpha Interferon, AS-101, bropirimine, acemannan, CL246,738, EL10, FP-21399, gamma interferon, granulocyte macrophage colony stimulating factor, IL-2, immune globulin intravenous, IMREG-1, IMREG-2, imuthiol diethyl dithio carbamate, alpha-2 interferon, methionine-enkephalin, MTP-PE, granulocyte colony stimulating sactor, remune, rCD4, recombinant soluble human CD4, interferon alfa-2, SK&F106528, soluble T4 yhymopentin, tumor necrosis factor (TNF), tucaresol, recombinant human interferon beta, and interferon alfa n-3.

Anti-infectives that may be used in combination with the compounds of the present invention include, but are not limited to, atovaquone, azithromycin, clarithromycin, trimethoprim, trovafloxacin, pyrimethamine, daunorubicin, clindamycin with primaquine, fluconazole, pastill, ornidyl, eflornithine pentamidine, rifabutin, spiramycin, intraconazole-R51211, trimetrexate, daunorubicin, recombinant human erythropoietin, recombinant human growth hormone, megestrol acetate, testerone, and total enteral nutrition.

Antifungals that may be used in combination with the compounds of the present invention include, but are not limited to, anidulafungin, C31G, caspofungin, DB-289, fluconzaole, itraconazole, ketoconazole, micafungin, posaconazole, and voriconazole.

Other compounds that may be used in combination with the compounds of the present invention include, but are not limited to, acmannan, ansamycin, LM 427, AR177, BMS-232623, BMS-234475, CI-1012, curdlan sulfate, dextran sulfate, STOCRINE EL10, hypericin, lobucavir, novapren, peptide T octabpeptide sequence, trisodium phosphonoformate, probucol, and RBC-CD4.

In addition, the compounds of the present invention may be used in combination with anti-proliferative agents for the treatment of conditions such as Kaposi's sarcoma. Such agents include, but are not limited to, inhibitors of metallo-matrix proteases, A-007, bevacizumab, BMS-275291, halofuginone, interleukin-12, rituximab, paclitaxel, porfimer sodium, rebimastat, and COL-3.

The particular choice of an additional agent or agents will depend on a number of factors that include, but are not limited to, the condition of the mammal being treated, the particular condition or conditions being treated, the identity of the compound or compounds of the present invention and the additional agent or agents, and the identity of any additional compounds that are being used to treat the mammal. The particular choice of the compound or compounds of the invention and the additional agent or agents is within the knowledge of one of ordinary skill in the art and can be made without undue experimentation.

The compounds of the present invention may be administered in combination with any of the above additional agents for the treatment of a mammal, such as a human, that is suffering from an infection with the HIV virus, AIDS, AIDS-related complex (ARC), or any other disease or condition which is related to infection with the HIV virus. Such a combination may be administered to a mammal such that a compound or compounds of the present invention are present in the same formulation as the additional agents described above. Alternatively, such a combination may be administered to a mammal suffering from infection with the HIV virus such that the compound or compounds of the present invention are present in a formulation that is separate from the formulation in which the additional agent is found. If the compound or compounds of the present invention are administered separately from the additional agent, such administration may take place concomitantly or sequentially with an appropriate period of time in between. The choice of whether to include the compound or compounds of the present invention in the same formulation as the additional agent or agents is within the knowledge of one of ordinary skill in the art.

Additionally, the compounds of the present invention may be administered to a mammal, such as a human, in combination with an additional agent that has the effect of increasing the exposure of the mammal to a compound of the invention. The term “exposure,” as used herein, refers to the concentration of a compound of the invention in the plasma of a mammal as measured over a period of time. The exposure of a mammal to a particular compound can be measured by administering a compound of the invention to a mammal in an appropriate form, withdrawing plasma samples at predetermined times, and measuring the amount of a compound of the invention in the plasma using an appropriate analytical technique, such as liquid chromatography or liquid chromatography/mass spectroscopy. The amount of a compound of the invention present in the plasma at a certain time is determined and the concentration and time data from all the samples are plotted to afford a curve. The area under this curve is calculated and affords the exposure of the mammal to the compound. The terms “exposure,” “area under the curve,” and “area under the concentration/time curve” are intended to have the same meaning and may be used interchangeably throughout.

Among the agents that may be used to increase the exposure of a mammal to a compound of the present invention are those that can as inhibitors, of at least one isoform of the cytochrome P450 (CYP450) enzymes. The isoforms of CYP450 that may be beneficially inhibited include, but are not limited to, CYP1A2, CYP2d6, CYP2C9, CYP2C19 and CYP3A4. Suitable agents that may be used to inhibit CYP 3A4 include, but are not limited to, delavirdine and ritonavir.

Such a combination may be administered to a mammal such that a compound or compounds of the present invention are present in the same formulation as the additional agents described above. Alternatively, such a combination may be administered such that the compound or compounds of the present invention are present in a formulation that is separate from the formulation in which the additional agent is found. If the compound or compounds of the present invention are administered separately from the additional agent, such administration may take place concomitantly or sequentially with an appropriate period of time in between. The choice of whether to include the compound or compounds of the present invention in the same formulation as the additional agent or agents is within the knowledge of one of ordinary skill in the art.

The present invention provides methods of treating a mammal co-infected with HIV and HCV, comprising administering to said mammal at least one compound of formula (I), or a pharmaceutically acceptable salt or solvate thereof, and at least one additional agent having anti-HCV activity. Among the at least one additional agents having anti-HCV activity, that are useful according to the present invention, is 2-[4-(2-{2-cyclopentyl-5-[(5,7-dimethyl[1,2,4]triazolo[1,5-a]pyrimidin-2-yl)methyl]-4-hydroxy-6-oxo-3,6-dihydro-2H-pyran-2-yl}ethyl)-2-fluorophenyl]-2-methylpropanenitrile or 2-[2-chloro-4-(2-{2-cyclopentyl-5-[(5,7-dimethyl[1,2,4]triazolo[1,5-a]pyrimidin-2-yl)methyl]-4-hydroxy-6-oxo-3,6-dihydro-2H-pyran-2-yl}ethyl)phenyl]-2-methylpropanenitrile, or a pharmaceutically acceptable salt or solvate of either. Furthermore, the at least one additional agent having anti-HCV activity includes, but is not limited to, those found in Table I, below.

TABLE I

| Agent name | Type of Inhibitor or Target | Source |
|---|---|---|
| Omega IFN | IFN-ù | BioMedicines Inc., Emeryville, |
| | | CA |
| BILN-2061 | serine protease inhibitor | Boehringer Ingelheim Pharma |
| | | KG, Ingelheim Germany |
| Summetrel | antiviral | Endo Pharmaceuticals |
| | | Holdings Inc., Chadds Ford, |
| | | PA |
| Roferon A | IFN αa2a | F. Hoffmann-LaRoche LTD, |
| | | Basel, Switzerland |
| Pegasys | PEGylated IFN-α2a | F. Hoffmann-LaRoche LTD, |
| | | Basel, Switzerland |
| Pegasys and Ribavirin | PEGylated IFN- | F. Hoffmann-LaRoche LTD, |
| | α2a/ribavirin | Basel, Switzerland |
| CellCept | HCV IgG | F. Hoffmann-LaRoche LTD, |
| | Immunosuppressant | Basel, Switzerland |
| Wellferon | Lymphoblastoid IFN-αn1 | GlaxoSmithKline plc, Uxbridge, |
| | | UK |
| A1 Buferon-α | Albumin IFN-α2b | Human Genome Sciences |
| | | Inc., Rockville MD |
| Levovirin | Ribavirin | ICN Pharmaceuticals, Cost |
| | | Mesa, CA |
| IDN-6556 | caspase inhibitor | Idun Pharmaceuticals Inc., |
| | | San Diego, CA |
| IP-501 | Antifibrotic | Indevus Pharmaceuticals Inc., |
| | | Lexington, MA |
| Actimmune | INF-γ | InterMune Pharmaceuticals |
| | | Inc., Brisbane, CA |
| Infergen A | IFN alfacon-1 | InterMune Pharmaceuticals |
| | | Inc., Brisbane, CA |
| ISIS 14803 | Antisense | ISIS Pharmaceuticals Inc., |
| | | Carlsbad, CA/Elan |
| | | Pharmaceuticals Inc., New |
| | | York, NY |
| Pegasys and Ceplene | pegylated IFN-α2a/ | Maxim Pharmaceuticals Inc., |
| | immune modulator | San Diego, CA |
| Ceplene | immune modulator | Maxim Pharmaceuticals inc., |
| | | San Diego, CA |
| Civacir | HCV lgG | Nabi Biopharmaceuticals Inc., |
| | immunosuppressant | Boca Raton, FL |
| Intron A and Zadaxin | IFN-α2b/α1-thymosin | RegeneRx Biopharmaceuticals |
| | | inc., Bethesda, MD/SciClone |
| | | Pharmaceuticals Inc., San |
| | | Mateo, CA |
| Levorvirin | IMPDH inhibitor | Ribapharm Inc., Cost Mesa, |
| | | CA |
| Viramidine | IMPDH inhibitor | Ribapharm Inc., Cost Mesa, |
| | | CA |
| Heptazyme | Ribozyme | Ribozyme Pharmaceuticals |
| | | Inc., Boulder, CO |
| Intron A | IFN-α2b | Schering-Plough Corporation, |
| | | Kenilworth, NJ |
| PEG-Intron | PEGylated IFN-α2b | Schering-Plough Corporation, |
| | | Kenilworth, NJ |
| Rebetron | IFN-α2b/ribavirin | Schering-Plough Corporation, |
| | | Kenilworth, NJ |
| Ribavirin | Ribavirin | Schering-Plough Corporation, |
| | | Kenilworth, NJ |
| PEG-Intron/Ribavirin | PEGylated IFN- | Schering-Plough Corporation, |
| | α2b/ribavirin | Kenilworth, NJ |
| Zadazim | Immune modulator | SciClone Pharmaceuticals |
| | | Inc., San Mateo, CA |
| Rebif | IFN-β1a | Serono, Genevea, Switzerland |
| IFN-β and EMZ701 | IFN-β and EMZ701 | Transition Therapeutics inc. |
| | | Ontario, Canada |
| T67 | β-tubulin inhibitor | Tularik Inc., South San |
| | | Francisco, CA |
| VX-497 | IMPDH inhibitor | Vertex Pharmaceuticals inc., |
| | | Cambridge, MA |
| VX-497 | IMPDH inhibitor | Vertex Pharmaceuticals inc., |
| | | Cambridge, MA/Eli Lilly and |
| | | Co. Inc., Indianapolis, IN |
| Omniferon | natural IFN-α | Viragen Inc., Plantation, FL |
| XTL-002 | Monoclonal antibody | XTL Biopharmaceuticals |
| R-803 | Polymerase (RdRp) | Rigel |
| | inhibitor | |
| NM283 | Nucleoside (RdRp) | Idenix |
| NM107 | inhibitor | |
| BMS-124 | NS5A inhibitors | BMS |
| ANA245 | TLR7 | Anadys |
| ANA971 | | |
| Actilon | TLR9 agonist | Coley Pharmaceutical |
| Zadaxin | Immune System | SciClone Pharmaceuticals |
| | | Inc., San Mateo, CA |
| Celgosivin (MBI-3253) | a-glucosidase | Micrologix Biotech |
| Compounds found in | Polymerase (RdRp) | Boehringer Ingelheim Pharma |
| WO03010140 | inhibitor | KG, Ingelheim Germany |
| Compound found in | Polymerase (RdRp) | Boehringer Ingelheim Pharma |
| WO03007945 | inhibitor | KG, Ingelheim Germany |
| Compounds found in | Polymerase (RdRp) | GlaxoSmithKline plc, Uxbridge, |
| WO03099801 | inhibitor | UK |
| 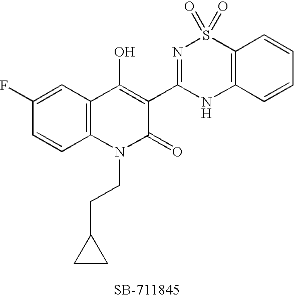 | Polymerase (RdRp) inhibitor | GlaxoSmithKline plc, Uxbridge, UK |
| 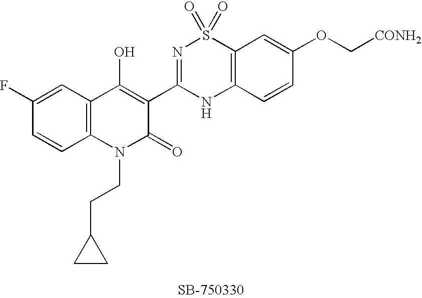 | Polymerase (RdRp) inhibitor | GlaxoSmithKline plc, Uxbridge, UK |
| Compounds found in | Polymerase (RdRp) | NeoGenesis, Inc. |
| WO03101993 | inhibitor | |
| Compounds found in | | Rigel |
| WO04018463 | | |
| Compounds found in | Polymerase (RdRp) | Wyeth, ViroPharma |
| WO03099275 | inhibitor | |
| 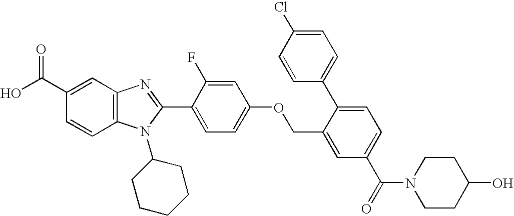 | Polymerase (RdRp) inhibitor | Japan Tabacco |
| 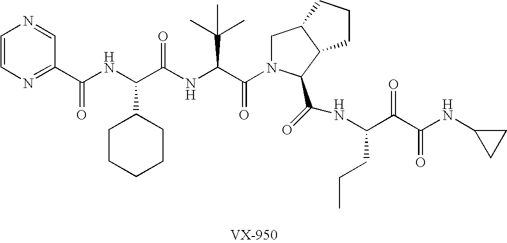 | Protease inhibitor | Vertex Pharmaceuticals inc., Cambridge, MA |
| 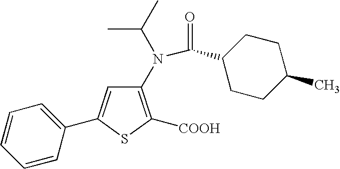 | Polymerase (RdRp) inhibitor | Shire Biochem |

Several different assay formats are available to measure integrase-mediated integration of viral DNA into target (or host) DNA and thus, identify compounds that modulate (e.g., inhibit) integrase activity. In general, for example, ligand-binding assays may be used to determine interaction with an enzyme of interest. When binding is of interest, a labeled enzyme may be used, wherein the label is a fluorescer, radioisotope, or the like, which registers a quantifiable change upon binding to the enzyme. Alternatively, the skilled artisan may employ an antibody for binding to the enzyme, wherein the antibody is labeled allowing for amplification of the signal. Thus, binding may be determined through direct measurement of ligand binding to an enzyme. In addition, binding may be determined by competitive displacement of a ligand bound to an enzyme, wherein the ligand is labeled with a detectable label. When inhibitory activity is of interest, an intact organism or cell may be studied, and the change in an organismic or cellular function in response to the binding of the inhibitory compound may be measured. Alternatively, cellular response can be determined microscopically by monitoring viral induced syncytium-formation (HIV-1 syncytium-formation assays), for example. Thus, there are various in vitro and in vivo assays useful for measuring HIV integrase inhibitory activity. See, e.g., Lewin, S. R. et al., Journal of Virology 73(7): 6099-6103 (July 1999); Hansen, M. S. et al., Nature Biotechnology 17(6):578-582 (June 1999); and Butler, S. L. et al., Nature Medicine 7(5): 631-634 (May 2001).

Exemplary specific assay formats used to measure integrase-mediated integration include, but are not limited to, ELISA, DELFIA® (PerkinElmer Life Sciences Inc. (Boston, Mass.)) and ORIGEN® (IGEN International, Inc. (Gaithersburg, Md.)) technologies. In addition, gel-based integration (detecting integration by measuring product formation with SDS-PAGE) and scintillation proximity assay (SPA) disintegration assays that use a single unit of double stranded-DNA (ds-DNA) may be used to monitor integrase activity.

In one embodiment of the invention, the preferred assay is an integrase strand-transfer SPA (stINTSPA) which uses SPA to specifically measure the strand-transfer mechanism of integrase in a homogenous assay scalable for miniaturization to allow high-throughput screening. The assay focuses on strand transfer and not on DNA binding and/or 3′ processing. This sensitive and reproducible assay is capable of distinguishing non-specific interactions from true enzymatic function by forming 3′ processed viral DNA/integrase complexes before the addition of target DNA. Such a formation creates a bias toward compound modulators (e.g., inhibitors) of strand-transfer and not toward compounds that inhibit integrase 3′ processing or prevent the association of integrase with viral DNA. This bias renders the assay more specific than known assays. In addition, the homogenous nature of the assay reduces the number of steps required to run the assay since the wash steps of a heterogenous assay are not required.

The integrase strand-transfer SPA format consists of 2 DNA components that model viral DNA and target DNA. The model viral DNA (also known as donor DNA) is biotinylated ds-DNA preprocessed at the 3′ end to provide a CA nucleotide base overhang at the 5′ end of the duplex. The target DNA (also known as host DNA) is a random nucleotide sequence of ds-DNA generally containing [³H]-thymidine nucleotides on both strands, preferably, at the 3′ ends, to enable detection of the integrase strand-transfer reaction that occurs on both strands of target ds-DNA.

Integrase (created recombinantly or synthetically and preferably, purified) is pre-complexed to the viral DNA bound to a surface, such as for example, streptavidin-coated SPA beads. Generally, the integrase is pre-complexed in a batch process by combining and incubating diluted viral DNA with integrase and then removing unbound integrase. The preferred molar ratio of viral DNA:integrase is about 1:about 5. The integrase/viral DNA incubation is optional, however, the incubation does provide for an increased specificity index with an integrase/viral DNA incubation time of about 15 to about 30 minutes at room temperature or at about 37° C. The preferred incubation is at about room temperature for about 15 minutes.

The reaction is initiated by adding target DNA, in the absence or presence of a potential integrase modulator compound, to the integrase/viral DNA beads (for example) and allowed to run for about 20 to about 50 minutes (depending on the type of assay container employed), at about room temperature or about 37° C., preferably, at about 37° C. The assay is terminated by adding stop buffer to the integrase reaction mixture. Components of the stop buffer, added sequentially or at one time, function to terminate enzymatic activity, dissociate integrase/DNA complexes, separate non-integrated DNA strands (denaturation agent), and, optionally, float the SPA beads to the surface of the reaction mixture to be closer in range to the detectors of, for example, a plate-based scintillation counter, to measure the level of integrated viral DNA which is quantified as light emitted (radiolabeled signal) from the SPA beads. The inclusion of an additional component in the stop buffer, such as for example CsCl or functionally equivalent compound, is optionally, and preferably, used with a plate-based scintillation counter, for example, with detectors positioned above the assay wells, such as for example a TopCount® counter (PerkinElmer Life Sciences Inc. (Boston, Mass.)). CsCl would not be employed when PMT readings are taken from the bottom of the plate, such as for example when a MicroBeta® counter (PerkinElmer Life Sciences Inc. (Boston, Mass.)) is used.

The specificity of the reaction can be determined from the ratio of the signal generated from the target DNA reaction with the viral DNA/integrase compared to the signal generated from the di-deoxy viral DNA/integrase. High concentrations (e.g., ≧50 nM) of target DNA may increase the d/dd DNA ratio along with an increased concentration of integrase in the integrase/viral DNA sample.

The results can be used to evaluate the integrase modulatory, such as for example inhibitory, activity of test compounds. For example, the skilled artisan may employ a high-throughput screening method to test combinatorial compound libraries or synthetic compounds. The percent inhibition of the compound may be calculated using an equation such as for example (1−((CPM sample−CPM min)/(CPM max−CPM min)))*100. The min value is the assay signal in the presence of a known modulator, such as for example an inhibitor, at a concentration about 100-fold higher than the IC₅₀ for that compound. The min signal approximates the true background for the assay. The max value is the assay signal obtained for the integrase-mediated activity in the absence of compound. In addition, the IC₅₀ values of synthetic and purified combinatorial compounds may be determined whereby compounds are prepared at about 10 or 100-fold higher concentrations than desired for testing in assays, followed by dilution of the compounds to generate an 8-point titration curve with ½-log dilution intervals, for example. The compound sample is then transferred to an assay well, for example. Further dilutions, such as for example, a 10-fold dilution, are optional. The percentage inhibition for an inhibitory compound, for example, may then be determined as above with values applied to a nonlinear regression, sigmoidal dose response equation (variable slope) using GraphPad Prism curve fitting software (GraphPad Software, Inc., San Diego, Calif.) or functionally equivalent software.

The stINTSPA assay conditions are preferably optimized for ratios of integrase, viral DNA and target DNA to generate a large and specific assay signal. A specific assay signal is defined as a signal distinguishing true strand-transfer catalytic events from complex formation of integrase and DNA that does not yield product. In other integrase assays, a large non-specific component (background) often contributes to the total assay signal unless the buffer conditions are rigorously optimized and counter-tested using a modified viral DNA oligonucleotide. The non-specific background is due to formation of integrase/viral DNA/target DNA complexes that are highly stable independent of a productive strand-transfer mechanism.

The preferred stINTSPA distinguishes complex formation from productive strand-transfer reactions by using a modified viral DNA oligonucleotide containing a di-deoxy nucleoside at the 3′ end as a control. This modified control DNA can be incorporated into integrase/viral DNA/target DNA complexes, but cannot serve as a substrate for strand-transfer. Thus, a distinct window between productive and non-productive strand-transfer reactions can be observed. Further, reactions with di-deoxy viral DNA beads give an assay signal closely matched to the true background of the assay using the preferred optimization conditions of the assay. The true background of the assay is defined as a reaction with all assay components (viral DNA and [³H]-target DNA) in the absence of integrase.

Assay buffers used in the integrase assay generally contain at least one reducing agent, such as for example 2-mercaptoethanol or DTT, wherein DTT as a fresh powder is preferred; at least one divalent cation, such as for example Mg⁺⁺, Mn⁺⁺, or Zn⁺⁺, preferably, Mg⁺⁺; at least one emulsifier/dispersing agent, such as for example octoxynol (also known as IGEPAL-CA or NP-40) or CHAPS; NaCl or functionally equivalent compound; DMSO or functionally equivalent compound; and at least one buffer, such as for example MOPS. Key buffer characteristics are the absence of PEG; inclusion of a high concentration of a detergent, such as for example about 1 to about 5 mM CHAPS and/or about 0.02 to about 0.15% IGEPAL-CA or functionally equivalent compound(s) at least capable of reducing non-specific sticking to the SPA beads and assay wells and, possibly, enhancing the specificity index; inclusion of a high concentration of DMSO (about 1 to about 12%); and inclusion of modest levels of NaCl (≦50 mM) and MgCl₂ (about 3 to about 10 mM) or functionally equivalent compounds capable of reducing the dd-DNA background. The assay buffers may optionally contain a preservative, such as for example NaN₃, to reduce fungal and bacterial contaminants during storage.

The stop buffer preferably contains EDTA or functionally equivalent compound capable of terminating enzymatic activity, a denaturation agent comprising, for example, NaOH or guanidine hydrochloride, and, optionally, CsCl or functionally equivalent compound capable of assisting in floating the SPA beads to the top of the assay container for scintillation detection at the top of the reservoir and, possibly, minimizing compound interference. An example of an integrase strand-transfer SPA is set forth in Example 13.

Alternatively, the level of activity of the modulatory compounds may be determined in an antiviral assay, such as for example an assay that quantitatively measures the production of viral antigens (e.g., HIV-1 p24) or the activities of viral enzymes (e.g., HIV-1 reverse transcriptase) as indicators of virus replication, or that measures viral replication by monitoring the expression of an exogenous reporter gene introduced into the viral genome (HIV-1 reporter virus assays) (Chen, B. K. et al., J. Virol. 68(2): 654-660 (1994); Terwilliger, E. F. et al., PNAS 86.3857-3861 (1989)). A preferred method of measuring antiviral activity of a potential modulator compound employs an HIV-1 cell protection assay, wherein virus replication is measured indirectly by monitoring viral induced host-cell cytopathic effects using, for example, dye reduction methods.

In one embodiment, the compounds of the present invention include those having an EC₅₀ value against HIV integrase of at least 10⁻⁵ M (or at least 10 μM) when measured with an HIV cell protection assay. In another embodiment are compounds of the present invention with an EC₅₀ value against HIV integrase of at least 1 μM when measured with an HIV cell protection assay. In yet another embodiment, the compounds of the present invention have an EC₅₀ against HIV integrase of at least 0.1 μM when measured with an HIV cell protection assay.

The inventive agents may be prepared using the reaction routes and synthesis schemes as described below, employing the techniques available in the art using starting materials that are readily available. The preparation of certain embodiments of the present invention is described in detail in the following examples, but those of ordinary skill in the art will recognize that the preparations described may be readily adapted to prepare other embodiments of the present invention. For example, the synthesis of non-exemplified compounds according to the invention may be performed by modifications apparent to those skilled in the art, e.g., by appropriately protecting interfering groups, by changing to other suitable reagents known in the art, or by making routine modifications of reaction conditions. Alternatively, other reactions disclosed herein or known in the art will be recognized as having adaptability for preparing other compounds of the invention.

General Procedures

The compounds of the present invention can be prepared directly from compound 1-1 (preferably a methyl or ethyl ester) and a substituted or unsubstituted hydroxylamine in the presence of a base, including but not limited to, for example, sodium hydroxide or sodium alkoxide in methanol or ethanol (Hauser, C. R. et al., Org. Synth. Coll. Vol. 2, p. 67, John Wiley, New York (1943)). Alternatively, the compound 1-1 can be saponified to the free acid 1-2 using lithium hydroxide or sodium hydroxide in methanol/water mixtures and heating the mixture to 100° C. in a SmithCreator® microwave for 1 to 5 min. Compound 1-2 can be coupled with a substituted or unsubstituted hydroxyl amine using a coupling reagent. Typical coupling reagents and conditions can be used, such as, for example, O-(azabenzotriazole-1-yl)-1,1,3,3-tetramethyl uronium hexafluorophosphate (HATU), N-(3-dimethylaminopropyl)-N′-ethylcarbodiimide (EDC) in DMF at ambient temperature, or many others that are familiar to those skilled in the art. Other suitable methods are described, for example, in M. B. Smith, J. March, Advanced Organic Chemistry, 5th edition, John Whiley & Sons, p. 508-511 (2001). The use of the preferred conditions described in this scheme would allow for parallel preparation or combinatorial libraries of such hydroxamates 1-3. 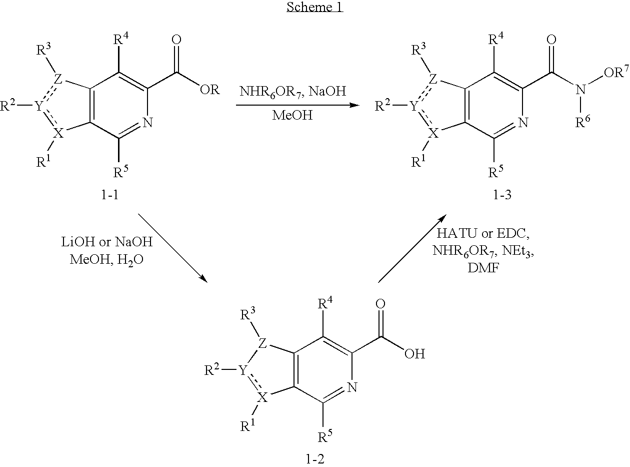
Preparation of Intermediates and Starting Materials

The precursors of type 1-1 with X=N, Y=C, Z=C (Compound 2-7) can be prepared from an arylsulfonyl or alkylsulfonyl protected pyrrole compound 2-2 formed from pyrrole compound 2-1 and an arylsulfonylchloride or an alkylsulfonylchloride in the presence of a base, such as, for example, triethylamine, using methods described, for example, in T. W. Greene, Protective Groups in Organic Chemistry, 3^{rd} edition, John Wiley & Sons, pp. 615-617 (1999). Reductive amination with a suitable substituted glycine ester compound 2-3 and a reducing agent, such as, for example, NaBH₃CN or NaBH(OAc)₃ (Abdel-Magid, A. F. et al., Tetrahedron Lett., 31, 5595-5598 (1990)) can provide the amine compound 2-4. Additional methods for reductive amination exist and are reviewed in C. F. Lane, Synthesis, p. 135 (1975). Titanium tetrachloride mediated cyclization (Dekhane, M. et al., Tetrahedron, 49, pp. 8139-8146 (1993); and Singh, S. K., Heterocycles, 44, pp. 379-391 (1997)) in a solvent, such as, for example, benzene or toluene, at the boiling temperature of the solvent can provide the arylsulfonyl or alkylsulfonyl protected precursor compound 2-5, which can be converted to the desired unprotected indole compound 2-6 using sodium alkoxide in alcohol (M. Dekhane, P. Potier, R. H. Dodd, Tetrahedron, 49, 8139-8146 (1993)). Alkylation of compound 2-6 with an alkylhalide in a polar solvent such as DMF or DMSO using sodium hydride as base (Eberle, M. K., J. Org. Chem. 41, pp. 633-636 (1976); Sundberg, R. J. et al., J. Org. Chem. 38, pp. 3324-3330 (1973)) can provide the desired precursor compound 2-7.

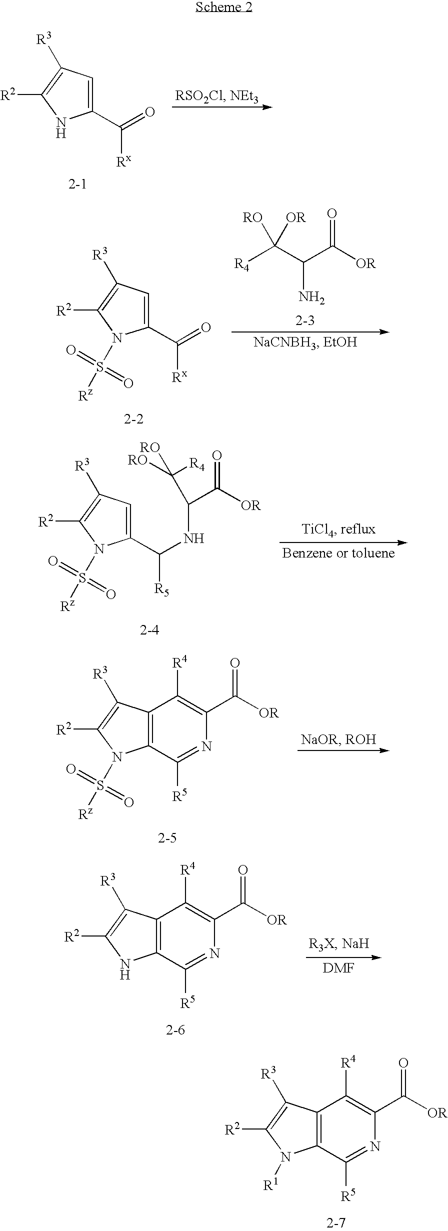

Scheme 3 depicts an alternative method for obtaining intermediate compound 2-5 adapted from the literature (Rousseau, J. F. et al., J. Org. Chem., 63, pp. 2731-2737 (1998) and citations therein) starting from the substituted pyrrole compound 3-1. The pyrrole nitrogen can be protected as a sulfonamide using the same methods described in Scheme 2. Addition of the anion of an N-Cbz glycine ester can provide the intermediate compound 3-4. Removal of the Cbz protecting group can be achieved using palladium catalyzed hydrogenation or other methods, such as those described in T. W. Greene, P. G. M. Wuts, Protective Groups in Organic Chemistry, 3^{rd} edition, John Wiley & Sons, pp. 531-537 (1999). Pictet-Spengler condensation followed by palladium catalyzed dehydrogenation in xylene can afford the intermediate compound 2-5.

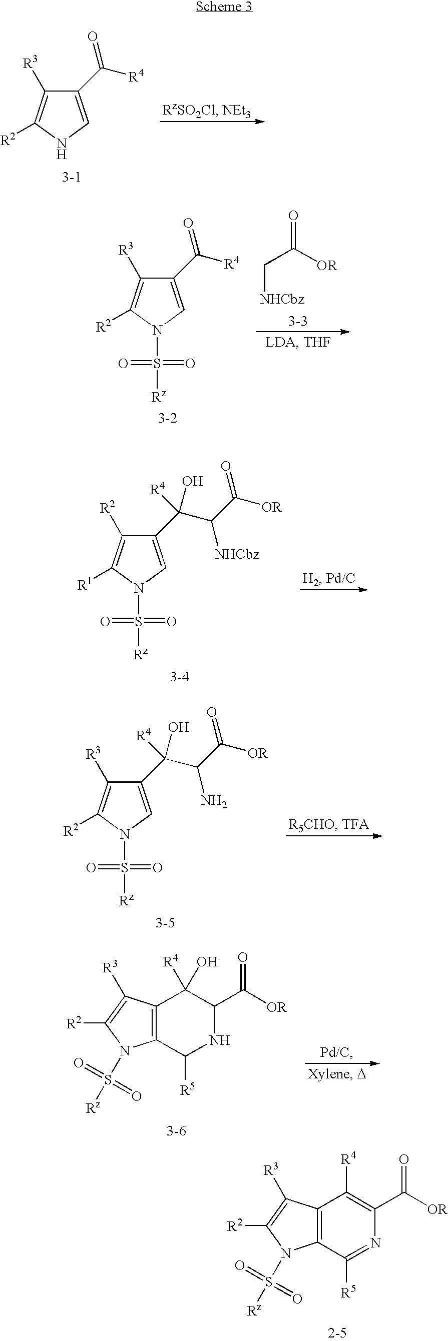

Scheme 4 depicts an alternative method for the formation of the azaindole core 4-9. The hydroxypyridine 4-1 can be converted to the corresponding triflate or bromide 4-2 using POBr₃ or trifluoromethanesulfonic anhydride and a base such as triethylamine. Reaction of 4-2 with zinc cyamide in the presence of a catalyst such as Pd(PPh₃)₄ (D. M. Tschaen et al. Synthetic Comm. 1994, 24, 887-890) can provide nitrile 4-3, which can be converted to ester 4-4 under acidic conditions. Reaction of 4-4 with dimethylformamide dimethyacetal followed by reduction can provide azaindole 4-6 (Prokopov, A. A. et al. Khim. Geterotsikl. Soedin. 1977, 1135, M. Sloan, R. S. Philipps, Bioorg. Med. Chem. Lett., 1992, 2, 1053-1056), which can be alkylated to 4-7 using a alkyl or benzyl halide and a base such as sodium hydride. Formylation of the pyrrole ring system in 4-7 can be accomplished using 1,1-dichloromethylmethyl ether in the presence of aluminum chloride as described by X. Doisy e. al. Bioorg. Med. Chem. 1999, 7, 921-932 to provide compound 4-8, which can react with an amine and a reducing agent such as sodium triacetoxy borohydride to provide 4-9.

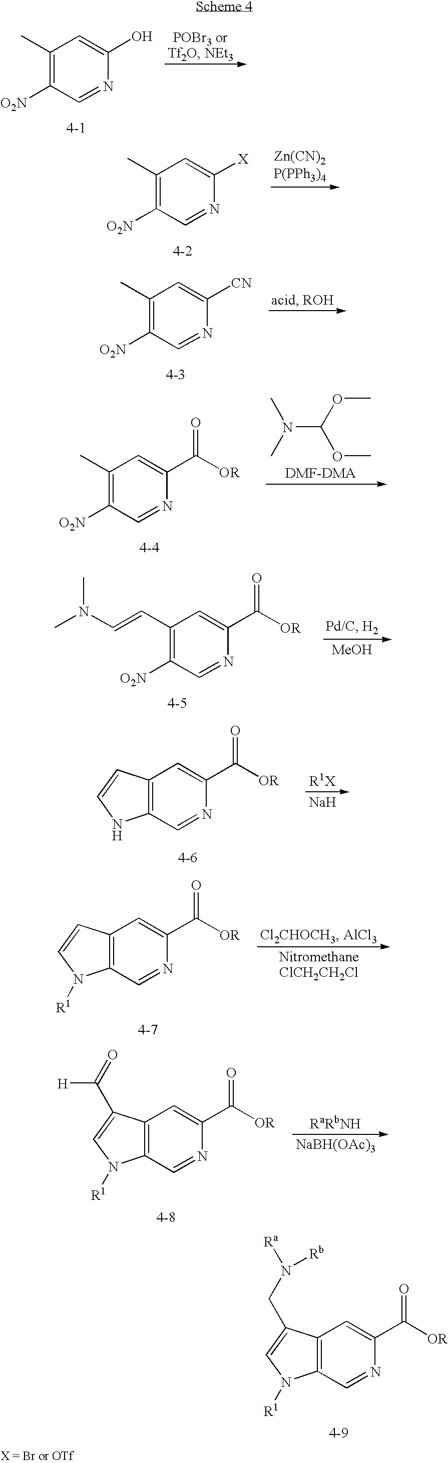

An alternative route that can provide 3-substituted pyrrolo[2,3-c]pyridines 5-6 and 5-7 from the unsubstituted precursor 5-1 is depicted in Scheme 5. Reaction of compound 5-1 with dimethylmethyleneimmonium chloride (A. P. Kozikowski, H. Ishida, Heterocycles 1980, 14, 55-58) can give the dimethylaminomethyl derivative 5-2. Alternatively, this step can be performed using classic Mannich reaction conditions (review: J. H. Brewster, E. L. Eliel, Org. Reactions, 1953, 7, 99). Upon treatment of 5-2 with sodium acetate and acetic anhydride in acetonitrile (J. N. Cocker, O. B. Mathre, W. H. Todd, J. Org. Chem., 1963, 28, 589-590) the corresponding acetate 5-3 can be obtained, which, on hydrolysis with a base such as potassium carbonate in methanol, can provide the precursor 5-5. Alkylation of the alcohol 5-5 can be achieved using an alkylhalide in the presence of a base such as sodium hydride in DMF as solvent to give 5-7. Alternatively, 5-2 can be treated with ethyl chloroformate (Shinohara, H.; Fukuda, T. and Iwao, M. Tetrahedron 1999, 55, 10989-11000) to form chloride 5-4 which can react with a thiol, alcohol, or amine to form 5-6 as described, in part, by Naylor, M. A. et al. J. Med. Chem. 1998, 41, 2720-2731.

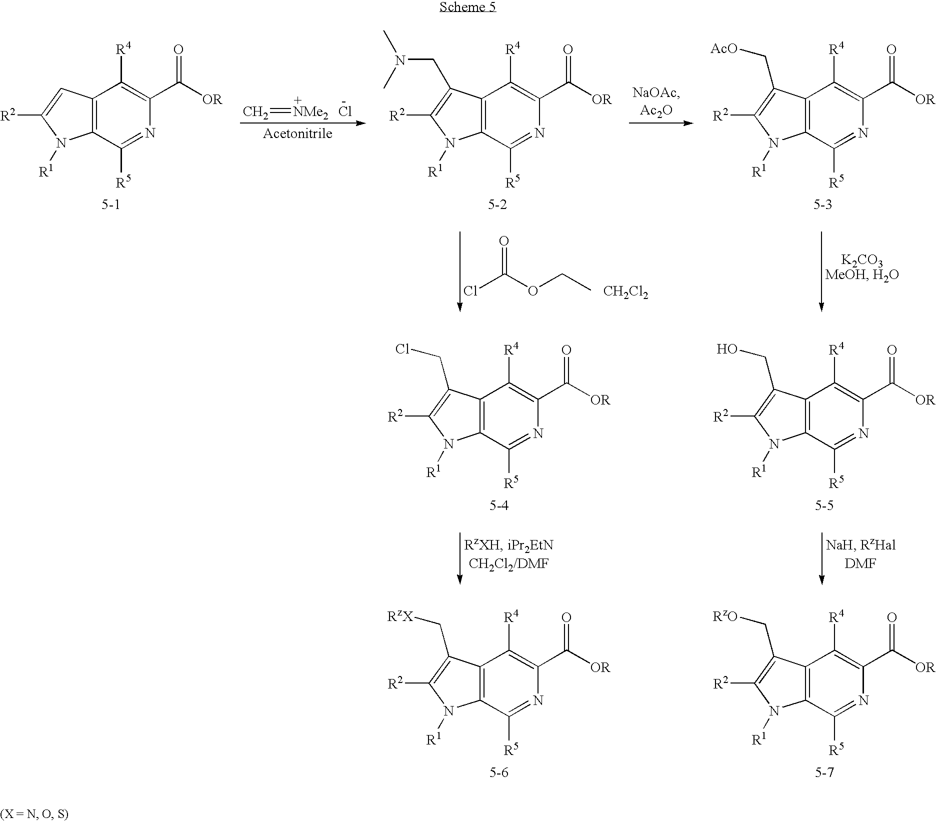

Imidazo[4,5-C]pyridine derivatives of type 1-1 (X=N, Y=C, Z=N) can be obtained according to Scheme 6. The histidine precursor 6-1 is commercially available or can be prepared according published methods (J. L. Kelley, C. A. Miller, E W. McLean, J. Med. Chem. 1977, 20, 721-723, G. Trout, J. Med. Chem. 1972, 15, 1259-1261). Pictet-Spengler reaction of 6-1 (F. Guzman et al., J. Med. Chem. 1984, 27, 564-570, M. Cain, F. Guzman, M. Cook, Heterocycles, 1982, 19, 1003-1007) can give the 1,2,3,4-tetrahydro-imidazo[4,5-c]pyridine-3-carboxylate 6-2, which can be converted to the methyl ester 6-3 via the corresponding acyl chloride or similar methods of ester formation known to those skilled in the art. Dehydrogenation to the unsaturated intermediate 6-4 can be achieved with selenium dioxide (J. G. Lee, K. C. Kim, Tetrahedron Lett, 1992, 33, 6363-6366), or a catalyst such as palladium or platinum in a solvent such as xylene at the boiling temperature of the solvent (D. Soerens et al. J. Org. Chem. 1979, 44, 535-545). Alkylation of 6-4 with an alkylhalide in the presence of a base such as sodium hydride similar to the methods described in Scheme 2 can provide the desired precursors as a mixture of regioisomers 6-5 and 6-6 that can be separated by column chromatography or other methods known to those skilled in the art.

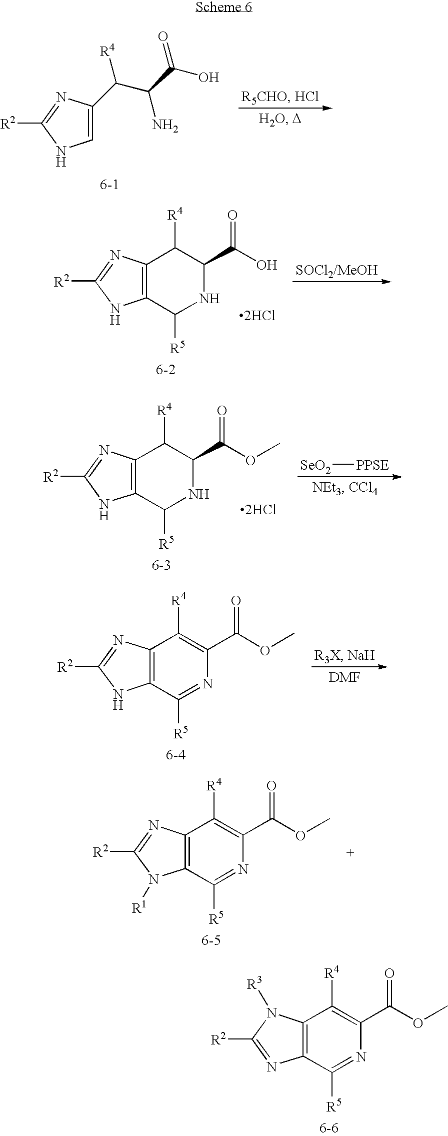

Scheme 7 sets forth a method for producing pyrrolo[3,2-c]pyridine derivatives 1-1 where X=C, Y=C, Z=N, and preferably R=an alkyl group (compound 7-3) via a substituted pyrrole compound of type 7-1 and a 2-azabutadiene compound of type 7-2 (Kantlehner, W. et al., Liebigs Ann. Chem., pp. 344-357 (1980)) under proton catalysis, following the procedures described in Biere, H. et al., Liebigs Ann. Chem., pp. 491-494 (1987). Friedel-Crafts acylation can provide ketone 7-5 which upon reduction with a reducing agent such as borane-t-butyl amine complex in THF can give compound 7-6 and alcohol 7-7.

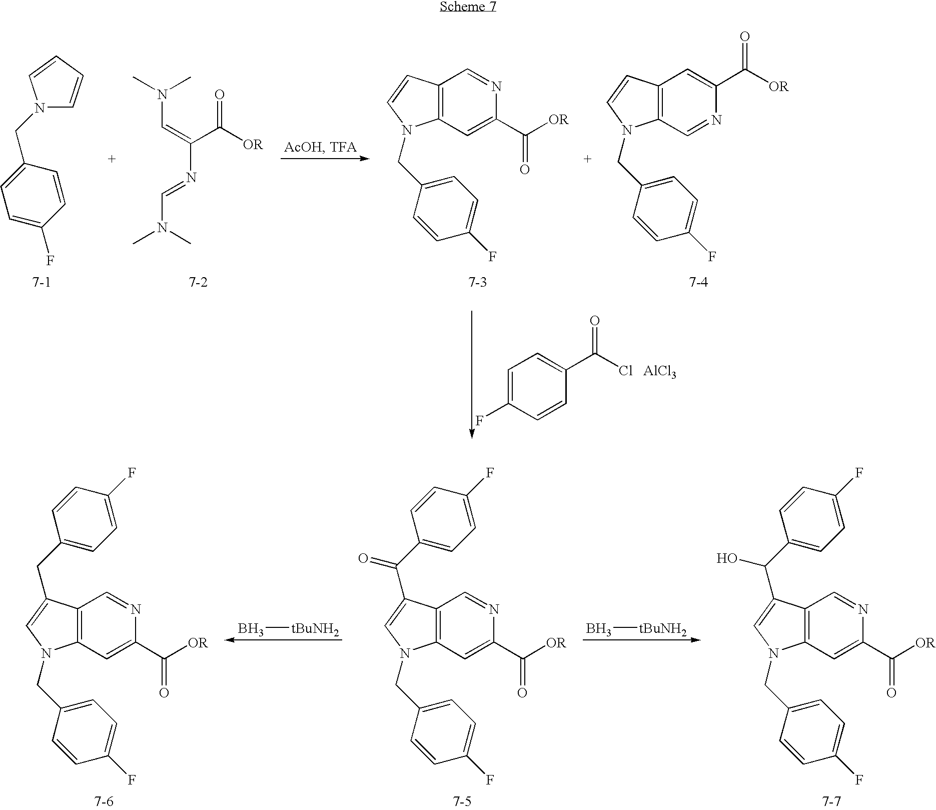

Scheme 8 depicts a general method (T. L. Gilchrist, C. W. Rees, J. A. R. Rodriguez, J. C. S. Chem. Comm. 1979, 627-628, L. Henn, D. M. B. Hickey, C. J. Moody, C. W. Rees, J. Chem. Soc. Perkin Trans. 1 1984, 2189-2196, A. Shafiee, H. Ghazar, J. Heterocyclic Chem. 1986, 23, 1171-1173) for the formation of compounds of general structure 1-1. Reaction of a substituted heteroaromatic aldehyde or ketone 8-1 with ethyl or methyl azidoacetate 8-2 in the presence of a base such as sodium hydride can provide azidocinnamate 8-3, which on thermolysis in boiling toluene or xylenes, can provide the desired product 8-4.

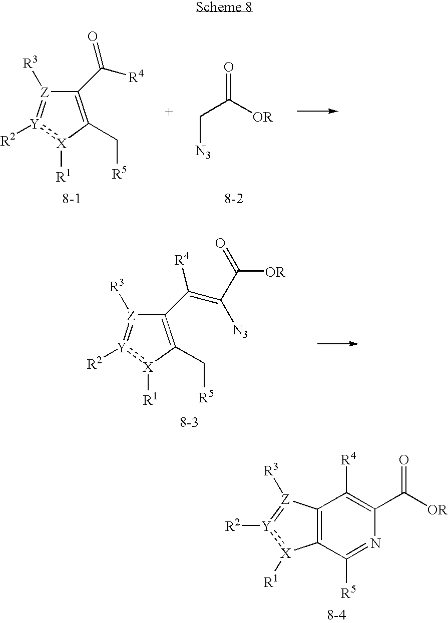

Another general method for formation the desired precursors (R⁵=H, Scheme 9) relies on the condensation of a dicarbonyl compound 9-1 with ethyl glycinate 9-2 (S. Mataka, K. Takahashi, M. Tashiro, J. Heterocyclic. Chem. 1981, 18, 1073-1075, R. P. Kreher, J. Pfister Chemiker-Zeitung 1984, 9, 275-277) that can provide a mixture of regioisomers 9-3 and 9-4, that can be separated by column chromatography or any other methods known to those skilled in the art.

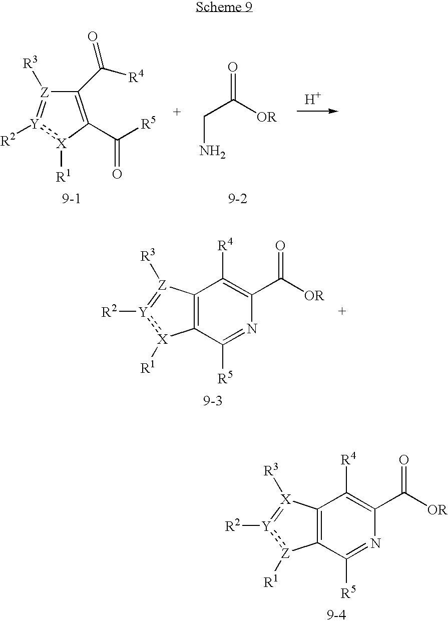

N-Alkylated hydroxylamines can be prepared by various methods described in the literature [for a review see H. J. Wroblowsky in Houben-Weyl, Methoden der Organischen Chemie, Suppl. Vol. E16, Part 1, Thieme, Stuttgart, N.Y., 1990, page 1-96. Scheme 11 describes a method developed by G. Doleschall, Tetrahedron Lett. 1987, 28, 2993-2994, which is based on N-alkylation of 3-methyl 5-hydroxy-4-isoxazole carboxylate 10-1 followed by treatment of 10-2 with hydrochloric acid. Another viable approach relies on the alkylation of bis-t-BOC hydroxylamine 10-4 followed by deprotection of the intermediate 10-5 with hydrochloric acid as described by M. A. Staszak C. W. Doecke, Tetrahedron Lett. 1994, 35, 6021-6024.

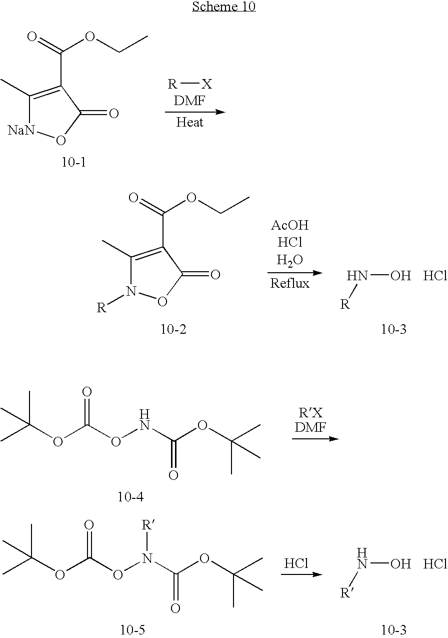

Scheme 11 shows a method for preparation of azaindazole 11-3 and 11-4 from 4-nitro-5-methylpyridine 11-1. Hydrogenation of 11-1 followed by treatment of the intermediate with sodium nitrite in acetic acid can provide azaindazole 11-2. This intermediate can be treated with 4-fluorobenzyl bromide and a base such as potassium carbonate to give both azaindazaole isomers 11-3 and 11-4, which can be separated by chromatography or other methods known to those of ordinary skill in the art. Alternative routes to 5-azaindazoles 11-3 and 11-4 have been described in the literature (Henn, L. J. Chem. Soc. Perkin Trans. 1 1984, 2189; Molina, P. Tetrahedron 1991, 47, 6737).

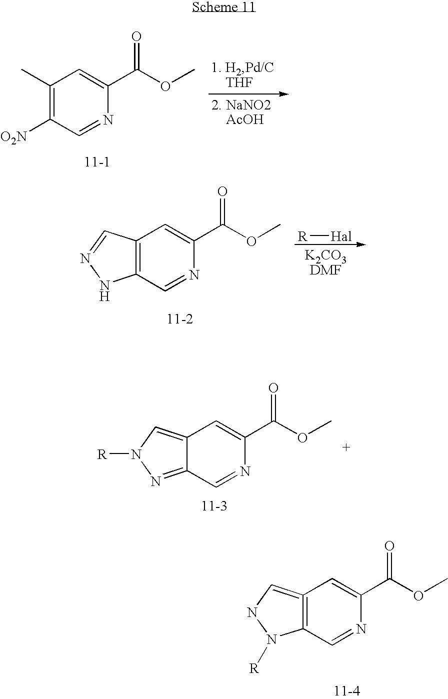

Scheme 12 depicts the synthesis of a 4-substituted azaindole 12-12. Ethyl 2-methyl-1H-pyrrole-3-carboxylate 12-1 (Wee, A. G. H.; Shu, A. Y. L.; Djerassi, C. J. Org. Chem. 1984, 49, 3327-3336) can be treated with a organo halide in the presence of a base such as NaH to provide pyrrole 12-3. Bromination using a bromine source such as NBS followed by radical bromination after the addition of a radical initiator such as benzoyl peroxide can give compound 12-4 which can react with a tosyl glycine ester 12-5 (Ginzel K. D., Brungs, P.; Steckan, E. Tetrahedron, 1989, 45, 1691-1701) to provide 12-6. Cyclization of 12-6 to 12-7 can be effected upon treatment with a base such as lithium hexamethyl disilazide. Catalytic hydrogenolysis (with e.g. Pd/C) can provide ester 12-8. Treatment of 12-8 with an organo halide and a base such as NaH can give 12-9. The hydroxy group in 12-8 can be converted to the triflate 12-10 using trifluoromethanesulfonic anhydride and a base such as triethyl amine. Triflate 12-10 can undergo palladium catalyzed couplings such as the Stille coupling with tributylstannylethene 12-11 in the presence of LiCl (J. K. Stille, Angew. Chem. 1986, 98, 504; Angew. Chem. Int. Ed. Engl. 1986, 25, 508; W. J. Scott, J. K. Stille, J. Am. Chem. Soc. 1986, 108, 3033; C. Amatore, A. Jutand, and A. Suarez J. Am. Chem. Soc. 1993, 115, 9531-9541) using a catalyst such Pd(PPh₃)₂Cl₂ (T. Sakamoto, C. Satoh, Y. Kondo, H. Yamanaka, Chem. Pharm. Bull. 1993, 41, 81-86).

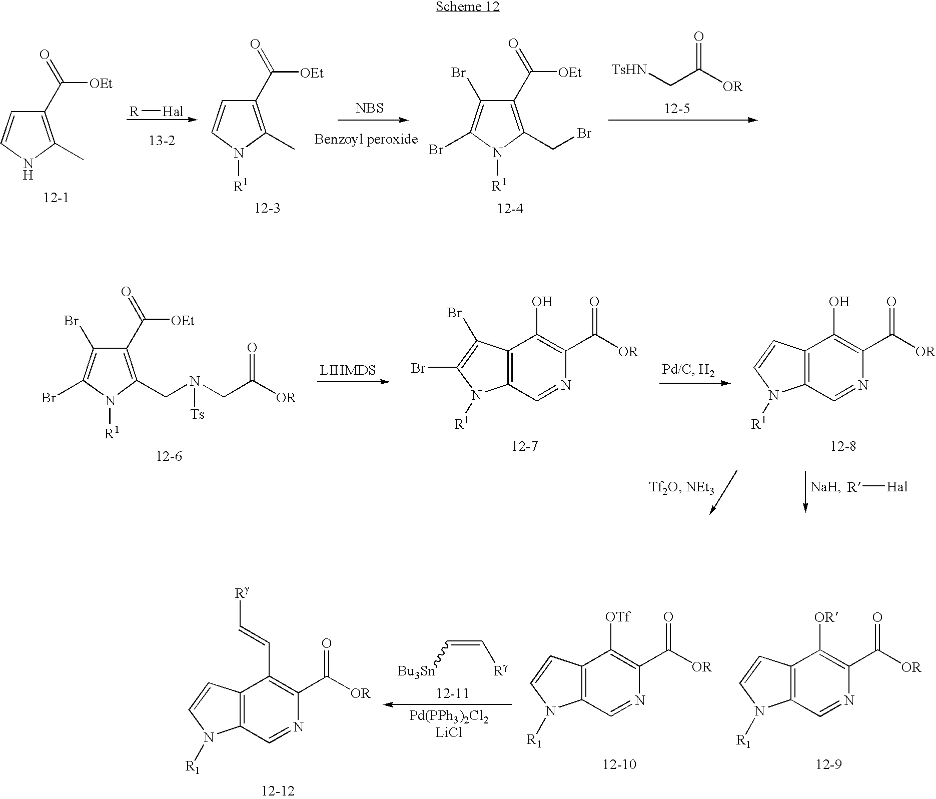

### EXAMPLES

The examples below are intended only to illustrate particular embodiments of the present invention and are not meant to limit the scope of the invention in any manner.

In the examples described below, unless otherwise indicated, all temperatures in the following description are in degrees Celsius (° C.) and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated.

The reactions set forth below were performed under a positive pressure of nitrogen, argon or with a drying tube, at ambient temperature (unless otherwise stated), in anhydrous solvents. Analytical thin-layer chromatography was performed on glass-backed silica gel 60° F. 254 plates (Analtech (0.25 mm)) and eluted with the appropriate solvent ratios (v/v). The reactions were assayed by high-pressure liquid chromotagraphy (HPLC) or thin-layer chromatography (TLC) and terminated as judged by the consumption of starting material. The TLC plates were visualized by UV, phosphomolybdic acid stain, or iodine stain.

Unless otherwise indicated, ¹H-NMR spectra were recorded on a Bruker instrument operating at 300 MHz and ¹³C-NMR spectra were recorded at 75 MHz. NMR spectra were obtained as DMSO-d6 or CDCl₃ solutions (reported in ppm), using chloroform as the reference standard (7.25 ppm and 77.00 ppm) or DMSO-d6 ((2.50 ppm and 39.52 ppm)). Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Infrared spectra were recorded on a Perkin-Elmer FT-IR Spectrometer as neat oils, as KBr pellets, or as CDCl₃ solutions, and when reported are in wave numbers (cm⁻¹). The mass spectra were obtained using LC/MS or APCI. All melting points are uncorrected.

All elemental analyses for compounds herein, unless otherwise specified, provided values for C, H, and N analysis that were within 0.4% of the theoretical value, and are reported as “C, H, N.”

In the following examples and preparations, “LDA” means lithium diisopropyl amide, “Et” means ethyl, “Ac” means acetyl, “Me” means methyl, “Ph” means phenyl, (PhO)₂POCl means chlorodiphenylphosphate, “HCl” means hydrochloric acid, “EtOAc” means ethyl acetate, “Na₂CO₃” means sodium carbonate, “NaOH” means sodium hydroxide, “NaCl” means sodium chloride, “NEt₃” means triethylamine, “THF” means tetrahydrofuran, “DIC” means diisopropylcarbodiimide, “HOBt” means hydroxy benzotriazole, “H₂O” means water, “NaHCO₃” means sodium hydrogen carbonate, “K₂CO₃” means potassium carbonate, “MeOH” means methanol, “i-PrOAc” means isopropyl acetate, “MgSO₄” means magnesium sulfate, “DMSO” means dimethylsulfoxide, “AcCl” means acetyl chloride, “CH₂Cl₂” means methylene chloride, “MTBE” means methyl t-butyl ether, “DMF” means dimethyl formamide, “SOCl₂” means thionyl chloride, “H₃PO₄” means phosphoric acid, “CH₃SO₃H” means methanesulfonic acid, “Ac₂O” means acetic anhydride, “CH₃CN” means acetonitrile, and “KOH” means potassium hydroxide.

### Example 1

### 1-(2,4-Difluorobenzyl)-N-hydroxy-3-{[3-(methylsulfonyl)pyrrolidin-1-yl]methyl}-1H-pyrrolo[2,3-c]pyridine-5-carboxamide

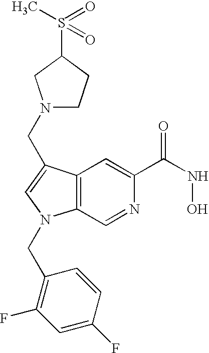

¹H NMR (MeOH-d₄) δ: 8.75 (s, 1H), 8.40 (s, 1H), 7.60 (s, 1H), 7.22 (m, 1H), 6.90-7.04 (m, 2H), 5.56 (s, 2H), 3.89 (m, 2H), 3.73 (m, 1H), 2.94 (d, 2H, J=7.16), 2.88 (s, 3H), 2.68-2.79 (b, 2H), 2.22 (b, 2H). HRMS calcd for C₂₁H₂₃F₂N₄O₄S (M+H⁺) 465.1408, found 465.1411. HPLC: >95% purity.

### Example 2

### 3-{[(2S)-2-(Aminocarbonyl)pyrrolidin-1-yl]methyl}-1-(2,4-difluorobenzyl)-N-hydroxy-1H -pyrrolo[2,3-c]pyridine-5-carboxamide

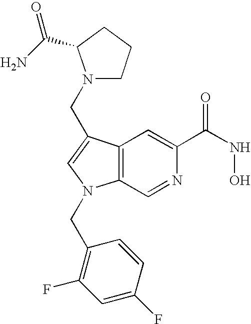

¹H NMR (MeOH-d₄) δ: 8.72 (s, 1H), 8.40 (s, 1H), 7.62 (s, 1H), 7.14 (m, 1H), 6.90-7.04 (m, 2H), 5.55 (s, 2H), 4.00 (s, 2H), 3.21 (m, 2H), 2.59 (m, 1H), 2.23 (m, 1H), 1.82 (b, 3H). HRMS calcd for C₂₁H₂₂F₂N₅O₃ (M+H⁺) 430.1691, found 430.1691. HPLC: >95% purity.

### Example 3

### 1-(2,4-Difluorobenzyl)-N-hydroxy-3-[(4-pyridin-2-ylpiperazin-1-yl)methyl]-1H-pyrrolo[2,3-c]pyridine-5-carboxamide

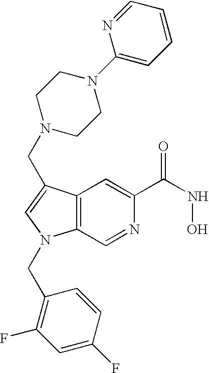

¹H NMR (MeOH-d₄) δ: 8.77 (s, 1H), 8.43 (s, 1H), 8.04 (m, 1H), 7.64 (s, 1H), 7.53 (m, 1H), 7.27 (m, 1H), 6.89-7.04 (m, 2H), 6.78 (d, 1H, J=8.47), 6.66 (m, 1H), 5.57 (s, 2H), 3.91 (m, 2H), 3.53 (m, 4H), 2.71 (m, 4H). HRMS calcd for C₂₅H₂₅F₂N₆O₂ (M+H⁺) 479.2007, found 479.1982. Anal. (C₂₅H₂₄F₂N₆O₂×1.2H₂O×0.1AcOH)C, H, N. HPLC: >95% purity.

### Example 4

### 1-(2,4-Difluorobenzyl)-3-(3,4-dihydroisoquinolin-2(1H)-ylmethyl)-N-hydroxy-1H-pyrrolo[2,3-c]pyridine-5-carboxamide

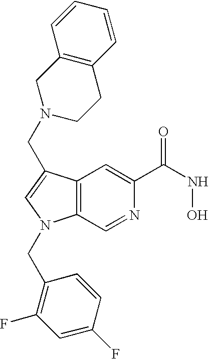

¹H NMR (MeOH-d₄) δ: 8.79 (s, 1H), 8.44 (s, 1H), 7.69 (s, 1H), 7.27 (m, 1H), 6.90-7.10 (m, 6H), 5.59 (s, 2H), 4.05 (s, 2H), 3.79 (s, 2H), 2.92 (s, 4H). HRMS calcd for C₂₅H₂₃F₂N₄O₂ (M+H⁺) 449.1789, found 449.1787. Anal. (C₂₅H₂₂F₂N₄O₂×0.8H₂O×0.1AcOH)C, H, N. HPLC: >95% purity.

### Example 5

### 3-{[4-(Aminocarbonyl)piperidin-1-yl]methyl}-1-(2,4-difluorobenzyl)-N-hydroxy-1H-pyrrolo[2,3-c]pyridine-5-carboxamide

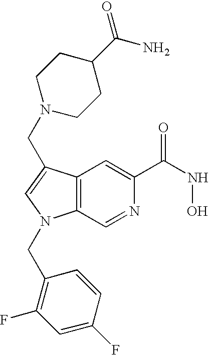

¹H NMR (MeOH-d₄) δ: 8.78 (s, 1H), 8.39 (s, 1H), 7.64 (s, 1H), 7.28 (m, 1H), 6.91-7.04 (m, 2H), 5.57 (s, 2H), 3.93 (s, 2H), 3.09 (m, 2H), 2.32 (m, 3H), 1.81 (m, 4H). HRMS calcd for C₂₂H₂₄F₂N₅O₃ (M+H⁺) 444.1847, found 444.1858. HPLC: >95% purity.

### Example 6

### 1-(2,4-Difluorobenzyl)-N-hydroxy-3-[(3-hydroxypyrrolidin-1-yl)methyl]-1H-pyrrolo[2,3-c]pyridine-5-carboxamide

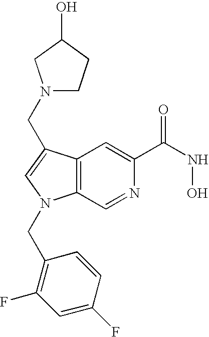

¹H NMR (MeOH-d₄) δ: 8.79 (s, 1H), 8.41 (s, 1H), 7.69 (s, 1H), 7.27 (m, 1H), 6.91-7.05 (m, 2H), 5.58 (s, 2H), 4.38 (b, 1H), 4.09 (m, 2H), 3.01 (b, 2H), 2.73-2.87 (m, 2H), 2.16 (m, 1H), 1.79 (b, 1H). HRMS calcd for C₂₀H₂₁F₂N₄O₃ (M+H⁺) 403.1582, found 403.1590. HPLC: >95% purity.

### Example 7

### 3-[(4-Acetylpiperazin-1-yl)methyl]-1-(2,4-difluorobenzyl)-N-hydroxy-1H-pyrrolo[2,3-c]pyridine-5-carboxamide

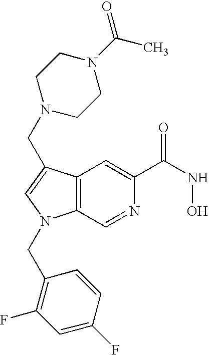

¹H NMR (MeOH-d₄) δ: 8.76 (s, 1H), 8.42 (s, 1H), 7.58 (s, 1H), 7.25 (m, 1H), 6.90-7.04 (m, 2H), 5.56 (s, 2H), 3.78 (s, 2H), 3.53 (b, 4H), 2.47 (b, 4H), 2.89 (s, 3H). HRMS calcd for C₂₂H₂₄F₂N₅O₃ (M+H⁺) 444.1847, found 444.1847. HPLC: >95% purity.

### Example 8

### 1-(2,4-Difluorobenzyl)-N-hydroxy-3-[(4-hydroxypiperidin-1-yl)methyl]-1H-pyrrolo[2,3-c]pyridine-5-carboxamide

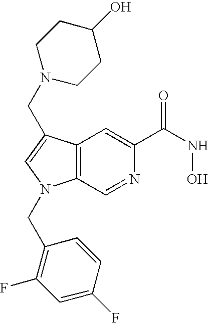

¹H NMR (MeOH-d₄) δ: 8.79 (s, 1H), 8.40 (s, 1H), 7.66 (s, 1H), 7.28 (m, 1H), 6.91-7.05 (m, 2H), 5.58 (s, 2H), 3.97 (s, 2H), 3.68 (m, 1H), 2.98 (m, 2H), 2.51 (m, 2H), 1.86 (m, 2H), 1.62 (m, 2H). HRMS calcd for C₂₁H₂₃F₂N₄O₃ (M+H⁺) 417.1738, found 417.1753. Anal. (C₂₁H₂₂F₂N₄O₃×0.4H₂O×0.7AcOH)C, H, N. HPLC: >95% purity.

### Example 9

### 3-{[3-(Aminocarbonyl)piperidin-1-yl]methyl}-1-(4-fluorobenzyl)-N-hydroxy-1H-pyrrolo[2,3-c]pyridine-5-carboxamide

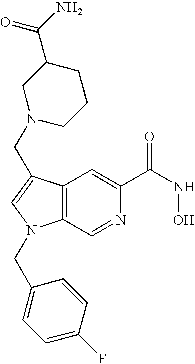

¹H NMR (MeOH-d₄) δ: 8.67 (s, 1H), 8.39 (s, 1H), 7.61 (s, 1H), 7.24 (m, 2H), 7.04 (m, 2H), 5.51 (s, 2H), 3.84 (s, 2H), 2.88 (m, 2H), 2.49 (m, 1H), 2.26-2.40 (m, 2H), 1.78 (m, 2H), 1.47-1.66 (m, 2H). HRMS calcd for C₂₂H₂₅F₂N₅O₃ (M+H⁺) 426.1941, found 426.1946. HPLC: >95% purity.

### Example 10

### 3-{[(2S)-2-(aminocarbonyl)pyrrolidin-1-yl]methyl}-1-(4-fluorobenzyl)-N-hydroxy-N-methyl-1H-pyrrolo[2,3-c]pyridine-5-carboxamide

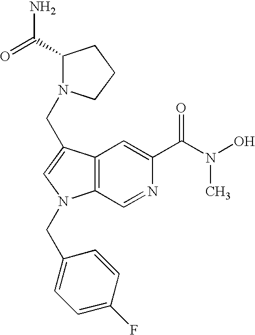

(a) Methyl 4-methyl-5-nitropyridine-2-carboxylate: HCl gas was bubbled into the solution of 2-cyano-4-methyl-5-nitropyridine (30 g) in methanol (200 mL) with cooling in a ice-water bath for 5 minutes. Then 3.3 mL water (1 eq.) was added to the flask. The resulting solution was heated to reflux for 3 hrs. The desired product precipitated as HCl salt (white crystals). The mixture was cooled to room temperature and the precipitate was collected by vacuum filtration. The solid was transferred to a 1 L separation funnel, neutralized with satd. aqueous NaHCO₃ (400 mL), and extracted with CH₂Cl₂. (400 mL). The organic layer was dried over Na₂SO₄, concentrated and dried in vacuum to give the title compound as white solid (33 g, 92% yield). ¹H NMR (DMSO-D₆) δ: 9.19 (s, 1H), 8.21 (s, 1H), 3.91 (s, 3H), 2.63 (s, 3H). LCMS (APCI, M+H⁺): 197.0.

### Example 11

### 3-{[(2S)-2-(aminocarbonyl)pyrrolidin-1-yl]methyl}-1-(4-fluorobenzyl)-N-hydroxy-1H -pyrrolo[2,3-c]pyridine-5-carboxamide

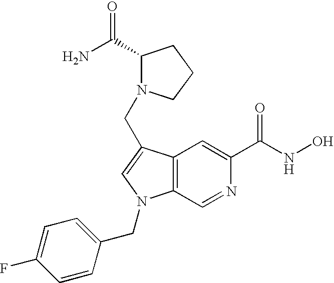

To a stirred solution of methyl 3-{[(2S)-2-(aminocarbonyl)pyrrolidin-1-yl]methyl}-1-(4-fluorobenzyl)-1H -pyrrolo[2,3-c]pyridine-5-carboxylate in MeOH (5 ml) was added 1M NaOH_{(aq)} (0.326 ml, 1 eq) and H₂NOH (0.400 ml, 20 eq, 50% wt. sol. in water). The resulting mixture was stirred for 16 hours at ambient temperature. The solvent was evaporated and the crude product was purified by prep-HPLC to provide the title compound as a white solid (0.0196 g, 15%). 1H NMR (300 MHz, MeOH) δ ppm 8.55 (s, 1H) 8.31 (d, J=0.94 Hz, 1H) 7.53 (s, 1H) 7.13 (dd, J=8.67, 5.27 Hz, 2H) 6.96 (t, J=8.76 Hz, 2H) 5.41 (s, 2H) 3.90 (s, 2H) 3.05-3.16 (m, 2H) 2.43-2.55 (m, 1H) 2.06-2.21 (m, 1H) 1.69-1.80 (m, 3H). LCMS (APCI, M+H⁺): 412.3. Anal. (C₂₁H₂₂FN₅O₃×1.1H₂O) C, H, N.

### Example 12

### 1-(4-fluorobenzyl)-N,4-dihydroxy-1H-pyrrolo[2,3-c]pyridine-5-carboxamide

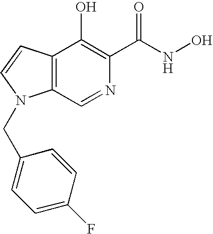

(a) Ethyl 1-(4-fluorobenzyl)-2-methyl-1H-pyrrole-3-carboxylate:

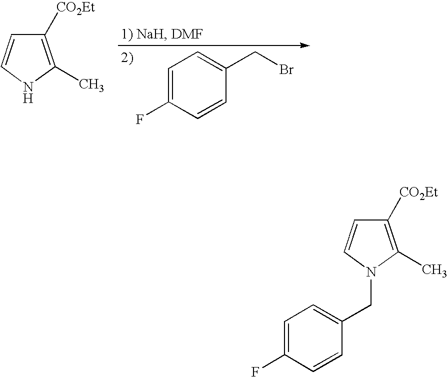

To a solution of ethyl 2-methyl-1H-pyrrole-3-carboxylate (106.26 g, 0.694 mol) (prepared by the method of: Wee, A. G. H.; Shu, A. Y. L.; Djerassi, C. J. Org. Chem. 1984, 49, 3327-3336) in anhydrous DMF (1.0 L), under nitrogen, was added sodium hydride (60% in oil, 30.5 g, 0.763 mol, 1.1 eq.) in 5 portions over 1 hour. When gas evolution ceased, 4-fluorobenzyl bromide (131.13 g, 0.694 mol) in anhydrous DMF (0.2 L) was added via pressure equalized addition funnel over 45 minutes. The mixture was allowed to stir at room temperature for 16 hours after the addition was complete, then was poured into water (1.4 L) in a 4 L separatory funnel. The mixture was extracted with diethyl ether (5×1.0 L) and the combined organic phases were washed with brine (3.0 L) and dried (Na₂SO₄). Filtration, rinsing of the filter cake with diethyl ether (0.5 L) and concentration in vacuo (house vacuum) gave the crude product and DMF. Residual DMF was removed on a cold finger trap rotary evaporator at full pump vacuum in a 40° C. water bath to give the crude benzylate pyrrole as an orange oil. The crude product was purified by chromatography on a column of silica gel (125 mm OD, 1 kg 230-400 mesh, packed with hexanes-EtOAc 95:5) eluted with hexanes:EtOAc (95:5, 2.0 L) and hexanes:EtOAc (90:10, 8.0 L) while collecting 500 mL fractions, using the flash technique. Fractions 4-18 were combined to afford ethyl 1-(4-fluorobenzyl)-2-methyl-1H-pyrrole-3-carboxylate (172.3 g, 95%) as a clear, pale yellow, viscous liquid.

TLC (Merck, hexanes:EtOAc 85:15, UV-+, cerium molybdate-+): R_{f}=0.26

LC-MS (Eclipse XDB-C8, 0.8 mL/min, gradient 80:20 to 5:95 H₂O (+0.1% HOAc):CH₃CN—5 minutes, APCI, + mode): RT− 3.711 min, m/e=262.1 (base), 263.2 (30)

¹H-NMR (300 MHz, CDCl₃): δ=1.33 (t, J=7.06 Hz, 3), 2.43 (s, 3), 4.26 (q, J=7.06 Hz, 2), 5.00 (s, 2), 6.52 (d, J=3.20 Hz, 1), 6.58 (d, J=3.20 Hz, 1), 6.92-7.04 (4).

(b) Ethyl 4,5-dibromo-2-(bromomethyl)-1-(4-fluorobenzyl)-1H-pyrrole-3-carboxylate

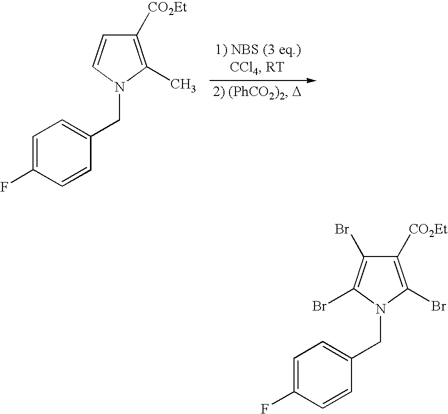

To NBS (267.5 g, 1.503 mol, 3 eq.) in anhydrous CCl₄ (0.5 L) in a 3 L, 3N round bottom flask, equipped with an internal temperature monitoring probe, addition funnel, and reflux condenser, was added ethyl 1-(4-fluorobenzyl)-2-methyl-1H-pyrrole-3-carboxylate (130.9 g, 0.501 mol) in anhydrous CCl₄ (0.5 L) over 15 minutes. The internal temperature rose to 43° C. during the addition and a transient red color developed, which faded upon completion of the addition. The mixture was allowed to stir for 15 minutes, then benzoyl peroxide (1.21 g, 5 mmol, 0.01 eq.) was added and the mixture was heated to an internal temperature of 77° C. (reflux) and maintained at that temperature for 1.5 hours. At this time point LC-MS (APCI) indicated complete reaction. The mixture was cooled to room temperature, the precipitated solid was removed by filtration, the filter cake was rinsed with CCl₄ (0.3 L), and the combined filtrates were concentrated in vacuo to give the crude tribromide as a red-brown semi-solid. The crude material was treated with dichloromethane (50 mL) and hexanes (250 mL) to produce a tan solid and a red-brown liquid. The solid was isolated by filtration, was rinsed with dichloromethane:hexanes (10:90, 0.5 L) and was dried in vacuo at room temperature to furnish 170.03 g (69%) of ethyl 4,5-dibromo-2-(bromomethyl)-1-(4-fluorobenzyl)-1H-pyrrole-3-carboxylate as a pale, tan solid. The filtrates were concentrated in vacuo to give a mother liquor that was purified by chromatography on a column of silica gel (70 mm OD, 400 g 230-400 mesh, hexanes:EtOAc 90:10, 250 mL fractions) using the flash technique. Fractions 3-6 provided an additional 29.57 g of ethyl 4,5-dibromo-2-(bromomethyl)-1-(4-fluorobenzyl)-1H-pyrrole-3-carboxylate as a pale, tan solid. Total: 199.6 g (81%). TLC (Merck, hexanes:EtOAc 90:10, UV-+, cerium molybdate-+): R_{f}=0.33. LC-MS (Eclipse XDB-C8, 0.8 mL/min, gradient 80:20 to 5:95 H₂O (+0.1% HOAc):CH₃CN—5 minutes, APCI, + mode): RT− 4.109 min, m/e=416.0 (50), 417.9 (base), 419 (50), M-Br. ¹H-NMR (300 MHz, CDCl₃): δ=1.39 (t, J=7.16 Hz, 3), 4.35 (q, J=7.16 Hz, 2), 4.77 (s, 2), 5.36 (s, 2), 6.96-7.07 (4).

(c) Ethyl 4,5-dibromo-1-(4-fluorobenzyl)-2-({(2-methoxy-2-oxoethyl)[(4-methylphenyl)sulfonyl]amino}methyl)-1H-pyrrole-3-carboxylate

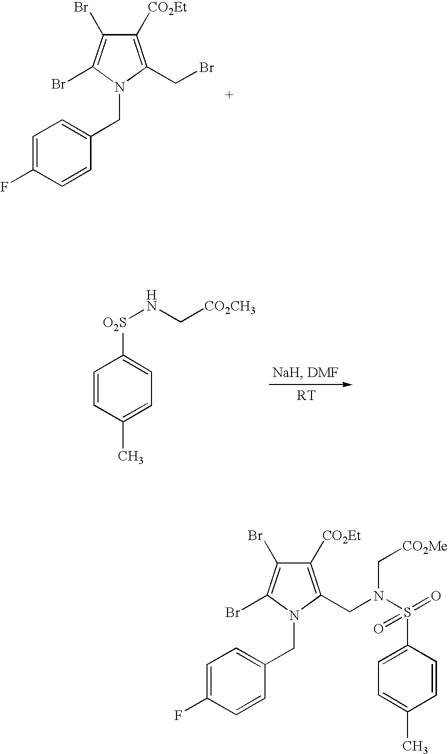

To a stirring solution of methyl N-[(4-methylphenyl)sulfonyl]glycinate (51.75 g, 0.213 mol, prepared by the method of: Ginzel, K. D.; Brungs, P.; Steckhan, E. Tetrahedron 1989, 45, 1691-1701) in anhydrous DMF (0.5 L) was added NaH (60% in oil, 8.59 g, 0.215 mol) in one portion. The mixture was allowed to stir for 30 minutes (warms and returns to room temperature) at which time a solution ethyl 4,5-dibromo-2-(bromomethyl)-1-(4-fluorobenzyl)-1H-pyrrole-3-carboxylate (105.92 g, 0.213 mol) in anhydrous DMF (0.5 L) was added over 1 hour. The mixture was allowed to stir at room temperature for 16 hours, then the DMF was removed in vacuo (full pump vacuum, 40° C. water bath) and the oily residue was dissolved in dichloromethane (0.75 L), the solution washed with saturated aq. NH₄Cl (0.5 L), brine (0.5 L0, and dried (Na₂SO₄). Filtration and concentration in vacuo provided the crude alkylated material as a viscous reddish oil. The crude material was heated in the presence of MeOH (0.75 L) until the MeOH was boiling, then dichloromethane was added slowly until solution was achieved. The red solution was cooled to room temperature (see off-white crystals) and the crystallization was completed by cooling in a refrigerator (4° C.) for 16 hours. The ivory solid was isolated by filtration, the solid was rinsed with diethyl ether:hexanes (0.5 L, 10:90) and the solid was dried in a vacuum oven (house vacuum, 50° C.) overnight to furnish ethyl 4,5-dibromo-1-(4-fluorobenzyl)-2-({(2-methoxy-2-oxoethyl)[(4-methylphenyl)sulfonyl]amino}methyl)-1H-pyrrole-3-carboxylate (108.2 g, 77%) as a free flowing, fine, ivory solid. TLC (Merck, hexanes:EtOAc 75:25, UV-+, cerium molybdate-+—purple): R_{f}=0.38. LC-MS (Eclipse XDB-C8, 0.8 mL/min, gradient 80:20 to 5:95 H₂O (+0.1% HOAc):CH₃CN—5 minutes, ESI, + mode): RT− 4.436 min, m/e=680.8 (55), 681.8 (18), 682.9 (base), 683.9 (30), 684.8 (62), 686.8 (10)—M+Na. ¹H-NMR (300 MHz, CDCl₃): δ=1.24 (t, J=7.16 Hz, 3), 2.14 (s, 3), 3.49 (s, 3), 3.89 (s, 2), 4.19 (q, J=7.16 Hz, 2), 4.55 (s, 2), 7.02 (d, J=2.45 Hz, 2), 7.04 (s, 2), 7.28 (d, J=8.19 Hz, 2), 7.59 (d, J=8.19 Hz, 2).

(d) Methyl 2,3-dibromo-1-(4-fluorobenzyl)-4-hydroxy-1 pyrrolo[2,3-c]pyridine-5-carboxylate

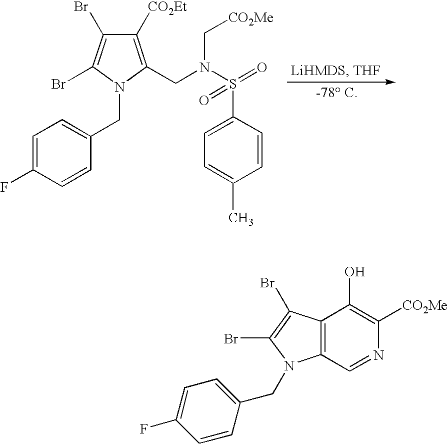

To solid LiHMDS (61.27 g, 0.366 mol), in a 3 L 3-neck round bottom flask equipped with a 0.5 L pressure equalized addition funnel and an internal temperature probe, was added anhydrous THF (0.5 L). The mixture was placed under nitrogen and immersed in a dry ice-i-PrOH bath. The solution was allowed to stir until the internal temperature reached −78° C. (1.25 h). To this stirring cold solution was added a solution of ethyl 4,5-dibromo-1-(4-fluorobenzyl)-2-({(2-methoxy-2-oxoethyl)[(4-methylphenyl)sulfonyl]amino}methyl)-1H-pyrrole-3-carboxylate (107.43 g, 0.163 mol) in anhydrous THF (0.5 L) at such a rate that the internal temperature does not exceed −70° C. (2 hours). During the course of the addition a yellow color was first noticed giving way to an orange/yellow solution, which then produced a precipitate and an orange/yellow solution. The reaction was allowed to stir for 30 minutes after the addition was complete, at which point HPLC/MS (sample taken at 15 minutes after addition) indicated the reaction was complete. The mixture was rapidly poured into a 6 L separatory funnel, which had been charged with saturated aq. NH₄Cl (1.5 L) and dichloromethane-methanol (95:5, 2 L). The mixture was rapidly shaken to distribute the reaction mixture and quench the reaction. The organic phase was separated, the aq. Layer was extracted with dichloromethane-methanol (95:5, 1 L) and the combined organic phases were filtered to remove a fine white precipitate, and then were dried (Na₂SO₄). Concentration in vacuo afforded the crude cyclized material as a yellow solid which was triturated with EtOH (0.6 L) and the resulting white solid was isolated by filtration, washed with anhydrous ethyl ether (50 mL) and dried in a vacuum oven (house vacuum, 50° C., 16 hours) to give 40.51 g (54.4%) of methyl 2,3-dibromo-1-(4-fluorobenzyl)-4-hydroxy-1H -pyrrolo[2,3-c]pyridine-5-carboxylate as a powdery white solid after drying in a vacuum oven. The filtrate was concentrated in vacuo and the residue was triturated with diethyl ether/hexanes (50:50, 0.25 L) to give 10.69 g (14.3%) of methyl 2,3-dibromo-1-(4-fluorobenzyl)-4-hydroxy-1H-pyrrolo[2,3-c]pyridine-5-carboxylate as a powdery white solid after drying in a vacuum oven (house vacuum, 50° C., 16 hours). The filtrate was again treated under the same conditions (0.1 L, 50:50 diethyl ether-hexanes) to give an additional 2.39 g (3.2%) for a total yield of 53.59 g (72%). TLC (Merck, CH₂Cl₂:EtOAc 50:50, UV-+, cerium molybdate-+): R_{f}=0.57. LC-MS (Eclipse XDB-C8, 0.8 mL/min, gradient 80:20 to 5:95 H₂O (+0.1% HOAc):CH₃CN—5 minutes, ESI, + mode): RT− 3.790 min, m/e=456.9 (55), 458.8 (base), 459.9 (15)—M+, 480.9—M+Na. ¹H-NMR (300 MHz, CDCl₃): δ=4.03 (s, 3), 5.48 (s, 2), 6.96-7.04 (2), 7.05-7.12 (2), 8.28 (s, 1), 11.60 (s, 1).

(e) Methyl 1-(4-fluorobenzyl)-4-hydroxy-1H-pyrrolo[2,3-c]pyridine-5-carboxylate

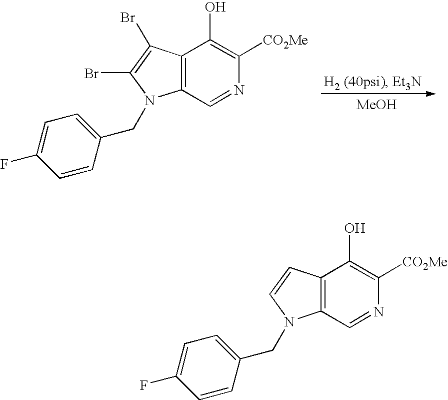

To a 2.5 L Parr flask was added methyl 2,3-dibromo-1-(4-fluorobenzyl)-4-hydroxy-1H-pyrrolo[2,3-c]pyridine-5-carboxylate (67.28 g, 0.147 mol), methanol (1.5 L) and triethyl amine (32.70 g, 0.323 mol, 2.2 eq.). Into this mixture was bubbled nitrogen for 10 minutes, then 10% Pd/C (15.6 g) was carefully added. The bottle was placed on a Parr apparatus, evacuated/purged with nitrogen (3×) and hydrogen was added to 40 psi. Shaking was commenced and after 5 minutes the pressure had gone to zero and the bottle was re-pressurized to 40 psi. This was repeated 2× at which point the pressure lowered to 35 psi and remained. TLC and LC/MS then indicated the reaction was complete (total time ca. 1 hour). The palladium was removed by filtration through a pad of celite, the filter cake was rinsed with dichloromethane (1.0 L) and the combined filtrates were concentrated in vacuo to give the crude product plus amine salts. The mixture was taken into EtOAc (2 L) and water (1.0 L), the organic phase was separated, the aqueous layer was extracted with EtOAc (0.6 L), and the combined organic phases were washed with brine (1.0 L) and dried (Na₂SO₄). Filtration and concentration in vacuo gave a powdery whit solid which was dried in a vacuum oven (house vacuum, 50° C., 16 hours) to afford 43.13 g (98%) of methyl 1-(4-fluorobenzyl)-4-hydroxy-1H-pyrrolo[2,3-c]pyridine-5-carboxylate as a free flowing, powdery white solid. TLC (Merck, CH₂Cl₂:EtOAc 50:50, UV-+, cerium molybdate-+): R_{f}=0.45 (fluorescent blue). LC-MS (Eclipse XDB-C8, 0.8 mL/min, gradient 80:20 to 5:95 H₂O (+0.1% HOAc):CH₃CN—5 minutes, APCI, + mode): RT− 3.217 min, m/e=301.1 (base, M+). ¹H-NMR (300 MHz, CDCl₃): δ=4.02 (s, 3), 5.36 (s, 2), 6.83 (d, J=3.10 Hz, 1), 6.96-7.04 (2), 7.07-7.13 (2), 7.17 (d, J=3.10 Hz, 1), 8.31 (s, 1), 11.40 (s, 1).

(f) 1-(4-Fluorobenzyl)-N,4-dihydroxy-1H-pyrrolo[2,3-c]pyridine-5-carboxamide. To methyl 1-(4-fluorobenzyl)-4-hydroxy-1H-pyrrolo[2,3-c]pyridine-5-carboxylate (0.22 g, 0.73 mmol) in methanol (10 mL) were added hydroxylamine (2 mL, 30.3 mmol, 50% in water) and sodium hydroxide (2.0 mL. 2.0 mmol, 1 N aqueous solution). The resulting solution was stirred for 16 h at ambient temperature. After addition of 1N hydrochloric acid (2.0 mL. 2.0 mmol) the product precipitated out. I was collected by was filtration, washed with water and ethyl acetate, and dried in vacuo to provide the title compound as a solid (0.18 g, 82% yield). ¹H NMR (DMSO-d₆) δ: 13.19 (s, 1H), 11.41 (s, 1H), 9.17 (s, 1H), 8.39 (s, 1H), 7.71 (d, 1H, J=3.1 Hz), 7.34 (t, 2H, J=8.8 Hz), 7.16 (t, 2H, J=8.8 Hz), 6.68 (d, 1H, J=3.1 Hz), 5.54 (s, 2H). LCMS (APCI, M+H⁺): 302.1. HRMS calcd for C₁₅H₁₂FN₃O₃ (M+H) 302.0936, found 302.0935. HPLC: 100% purity.

### Example 13

### 1-(4-Fluorobenzyl)-N-hydroxy-4-methoxy-N-methyl-1H-pyrrolo[2,3-c]pyridine-5-carboxamide

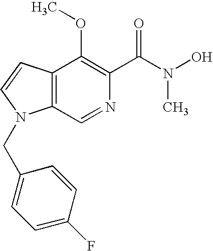

(a) Methyl 1-(4-fluorobenzyl)-4-methoxy-1H-pyrrolo[2,3-c]pyridine-5-carboxylate. To methyl 1-(4-fluorobenzyl)-4-hydroxy-1H-pyrrolo[2,3-c]pyridine-5-carboxylate (0.25 g, 0.83 mmol) in DMF (10 mL) were added sodium hydride (0.037 g, 0.92 mmol, 60% in mineral oil) and iodomethane (0.057 mL. 0.92 mmol). The solution was stirred for 3 h at ambient temperature. Then reaction mixture was quenched with saturated aqueous ammonium chloride solution (10 mL), and extracted with ethyl acetate (3×50 mL). The organic extracts were washed with brine (3×50 mL), dried over sodium 10, sulfate, concentrated in vacuo and purified by flash chromatography. Elution with ethyl acetate provided the title compound as a solid (0.10 g, 38% yield). ¹H NMR (CD₃OD) δ: 8.43 (s, 1H), 7.61 (d, 1H, J=3.1 Hz), 7.24 (t, 2H, J=8.8 Hz), 7.05 (t, 2H, J=8.8 Hz), 6.92 (d, 1H, J=3.1 Hz), 5.52 (s, 2H), 4.16 (s, 3H), 3.91 (s, 3H). LCMS (APCI, M+H⁺): 315.0.

(b) 1-(4-Fluorobenzyl)-4-methoxy-1H-pyrrolo[2,3-c]pyridine-5-carboxylic acid. The title compound was prepared by hydrolysis of methyl 1-(4-fluorobenzyl)-4-methoxy-1H-pyrrolo[2,3-c]pyridine-5-carboxylate in a manner similar to step (b) of example 1. ¹H NMR (DMSO-d₆): [symbol] : 8.30 (s, 1H), 7.65 (d, 1H, J=3.1 Hz), 7.24 (t, 2H, J=8.8 Hz), 7.14 (t, 2H, J=8.8 Hz), 6.58 (d, 1H, J=3.1 Hz), 5.47 (s, 2H), 3.92 (s, 3H). LCMS (APCI, M+H⁺): 301.1.

(c) 1-(4-Fluorobenzyl)-N-hydroxy-4-methoxy-N-methyl-1H-pyrrolo[2,3-c]pyridine-5-carboxamide. The title compound was prepared by coupling of 1-(4-fluorobenzyl)-4-methoxy-1H-pyrrolo[2,3-c]pyridine-5-carboxylic acid with N-methyl hydroxylamine hydrochloride in a manner similar to step (c) of example 1. ¹H NMR (DMSO-d₆) [symbol] : 9.70 (s, 1H), 8.50 (s, 1H), 7.76 (s, 1H), 7.33 (m, 2H), 7.16 (d, 2H, J=8.9 Hz), 6.76 (s, 1H), 5.51 (s, 2H), 4.00 (s, 3H), 2.96 (s, 3H). LCMS (APCI, M+H⁺): 330.1. HRMS calcd for C₁₇H₁₇FN₃O₃ (M+H) 330.1249, found 330.1250. HPLC: 98% purity.

### Example 14

### 3-{[(2S)-2-(Aminocarbonyl)pyrrolidin-1-yl]methyl}-1-(2,4-difluorobenzyl)-N-hydroxy-N-methyl-1H-pyrrolo[2,3-c]pyridine-5-carboxamide

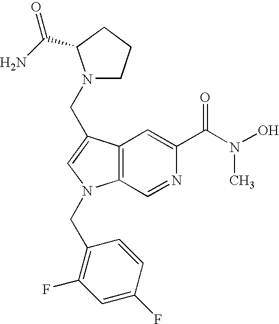

¹H NMR (MeOH-d₄) δ: 8.67 (s, 1H), 8.19 (s, 1H), 7.60 (s, 1H), 7.10-7.15 (m, 1H), 6.82-6.95 (m, 2H), 5.47 (s, 2H), 3.88 (s, 2H), 3.21 (s, 3H), 3.04-3.08 (m, 2H), 2.44-2.49 (m, 1H), 2.10-2.16 (m, 1H), 1.71-1.73 (m, 2H). LC/MS (API-ES, M+H⁺): 441.1. HRMS calcd for C₂₂H₂₃F₂N₅O₃ (M+H⁺) 444.1842, found 444.1854. HPLC: 100% purity.

### Example 15

### 1-(4-Fluorobenzyl)-N-hydroxy-3-{[3-(methylsulfonyl)pyrrolidin-1-yl]methyl}-1H-pyrrolo[2,3-c]pyridine-5-carboxamide

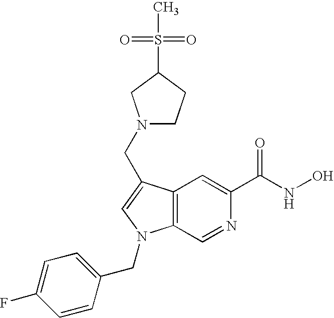

(a) Methyl 1-(4-fluorobenzyl)-3-{[3-(methylsulfonyl)pyrrolidin-1-yl]methyl}-1/pyrrolo[2,3-c]pyridine-5-carboxylate. A solution of methyl 1-(4-fluorobenzyl)-3-formyl-1H-pyrrolo[2,3-c]pyridine-5-carboxylate (624 mg, 2 mmol, 1.0 eq, in 8 mL anhydrous methanol) was mixed with a solution of 3-(methylsulfonyl)pyrrolidine (298 mg, 2 mmol, 1.0 eq, in 8 mL anhydrous methanol). Molecular sieves 4 Å (1 g) and sodium cyanoborohydride (628 mg, 10 mmol, 5 eq) were added, and the resulting mixture was stirred at room temperature overnight. The reaction was then filtered through celite, and the filtrate was concentrated to dryness under reduced pressure. The residue was subjected to flash chromatography with gradient elution to afford the title compound (316 mg, 36% yield). LC/MS [APCI, (M+H)⁺]: 446.40.

(b) 1-(4-Fluorobenzyl)-N-hydroxy-3-{[3-(methylsulfonyl)pyrrolidin-1-yl]methyl}-1 pyrrolo[2,3-c]pyridine-5-carboxamide. Methyl 1-(4-fluorobenzyl)-3-{[3-(methylsulfonyl)pyrrolidin-1-yl]methyl}-1H-pyrrolo[2,3-c]pyridine-5-carboxylate from (a) (150 mg, 0.34 mmol, 1 eq) was dissolved in 8 mL methanol. Then 1.36 mL 0.5 M NaOH (0.68 mmol, 2 eq) and 0.5 mL 50% aqueous hydroxylamine were added. The resulting reaction mixture was stirred at roomtemp. overnight. The solvent was removed under reduced pressure, and the resulting residue was purified by prep HPLC to afford the title compound (66 mg, 43.5% yield). ¹HNMR (300 MHz, MeOH-d₄) δ (ppm): 8.76 (s, 1H), 8.45 (s, 1H), 7.95 (s, 1H), 7.30-7.17 (m, 2H), 7.08-6.93 (m, 2H), 5.52 (s, 2H), 4.65 (s, 2H), 4.17-4.02 (m, 1H), 3.92-3.65 (m, 2H), 3.57-3.38 (b, 2H), 2.97 (s, 3H), 2.58-2.35 (m, 2H). HRMS calcd for C₂₁H₂₄N₄O₄FS (M+H⁺) 447.1502, found 447.1508. HPLC: 99% purity.

### Example 16

### 1-(4-Fluorobenzyl)-N-hydroxy-3-[(4-hydroxypiperidin-1-yl)methyl]-1H-pyrrolo[2,3-c]pyridine-5-carboxamide

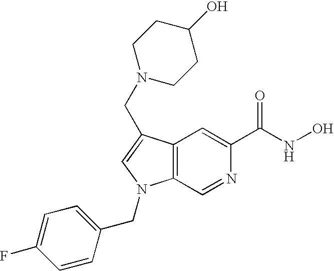

(a) Methyl 1-(4-fluorobenzyl)-3-[(4-hydroxypiperidin-1-yl)methyl]-1H-pyrrolo[2,3-c]pyridine-5-carboxylate. A solution of methyl 1-(4-fluorobenzyl)-3-formyl-1H-pyrrolo[2,3-c]pyridine-5-carboxylate (312 mg, 1.0 mmol, 1.0 eq) in 4 mL anhydrous dichloromethane was mixed with a solution of piperidin-4-ol (101 mg, 1.0 mmol, 1.0 eq) in 4 mL dichloromethane. After the reaction mixture was stirred under an atmosphere of nitrogen for 1 h at room temp., sodium triacetoxyborohydride (530 mg, 2.5 mmol, 2.5 eq.) was added, and the resulting mixture was stirred at room temp. overnight. The solvent was removed under reduced pressure, and the residue was dissolved in 8 mL 6:3:1 mixture solvent of ethylacetate/dichloromethane/methanol. The organic phase washed with 8 mL 1.0 M aqueous potassium carbonate solution, and the aqueous layer was extracted with the 6:3:1 mixture of ethylacetate/dichloromethane/methanol (2×8 mL). The organic phases were combined, dried over anhydrous sodium sulfate, and concentrated to dryness under reduced pressure to afford the title compound without further purification. ¹HNMR (300 MHz, MeOH-d₄) δ (ppm): 8.61 (s, 1H), 8.42 (s, 1H), 7.55 (s, 1H), 7.19-7.10 (m, 2H), 6.97-6.88 (m, 2H), 5.41 (s, 2H), 4.75 (s, 3H), 3.82 (s, 2H), 3.55-3.45 (m, 1H), 2.81-2.66 (m, 2H), 2.25-2.07 (m, 2H), 1.77-1.66 (m, 2H), 1.51-1.36 (m, 2H).

(b) 1-(4-Fluorobenzyl)-N-hydroxy-3-[(4-hydroxypiperidin-1-yl)methyl]-1H-pyrrolo[2,3-c]pyridine-5-carboxamide. The title compound was prepared from methyl 1-(4-fluorobenzyl)-3-[(4-hydroxypiperidin-1-yl)methyl]-1H-pyrrolo[2,3-c]pyridine-5-carboxylate using the method in example 26. ¹H NMR (MeOH-d₄) δ: 8.78 (s, 1H), 8.45 (s, 1H), 7.73 (s, 1H), 7.33-7.29 (m, 2H), 7.10-7.06 (m, 2H), 5.58 (s, 2H), 4.22 (b, 2H), 3.74 (b, 1H), 3.26 (b, 2H), 2.84 (b, 2H), 1.86 (b, 2H), 1.64 (b, 2H) LCMS (APCI, M+H⁺): 399.15. HPLC: 96% purity.

### Example 17

### 1-(4-Fluorobenzyl)-N-hydroxy-3-[(4-hydroxypiperidin-1-yl)methyl]-N-methyl-1H-pyrrolo[2,3-c]pyridine-5-carboxamide

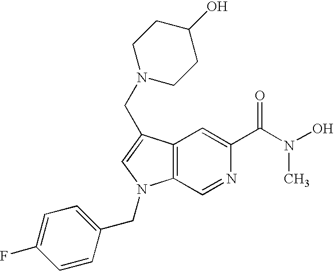

(a) 1-(4-Fluorobenzyl)-3-[(4-hydroxypiperidin-1-yl)methyl]-1H-pyrrolo[2,3-c]pyridine-5-carboxylic acid ¹H NMR (DMSO-d₆) δ: 8.90 (s, 1H), 8.39 (s, 1H), 7.76 (s, 1H), 7.35-7.33 (m, 2H), 7.18-7.14 (m, 2H), 5.55 (s, 2H), 3.63 (s, 2H), 3.41-3.39 (m, 1H), 2.72-2.68 (m, 2H), 2.07-2.02 (m, 2H), 1.69-1.65 (m, 2H), 1.37-1.33 (m, 2H). LCMS (APCI, M+H⁺): 384.15. HPLC: 99% purity.

(b) 1-(4-Fluorobenzyl)-N-hydroxy-3-[(4-hydroxypiperidin-1-yl)methyl]-N-methyl-1H-pyrrolo[2,3-c]pyridine-5-carboxamide. ¹H NMR (MeOH-d₄) δ: 8.78 (s, 1H), 8.31 (b, 1H), 7.80 (s, 1H), 7.33-7.30 (m, 2H), 7.11-7.05 (m, 2H), 5.57 (s, 2H), 4.10 (b, 2H), 3.74 (m, 1H), 3.43 (s, 3H), 3.12-3.07 (m, 2H), 2.67 (b, 2H), 1.94-1.86 (m, 2H), 1.86-1.60 (m, 2H). LCMS (APCI, M+H⁺): 413.05. HPLC: 98% purity.

### Example 18

### 1-(4-fluorobenzyl)-N-hydroxy-3-[(3-hydroxypyrrolidin-1-yl)methyl]-N-methyl-1H-pyrrolo[2,3-c]pyridine-5-carboxamide

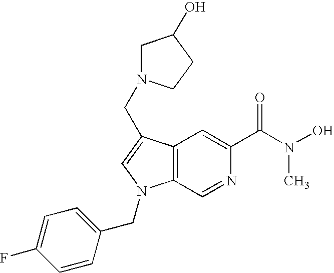

(a) Methyl 1-(4-fluorobenzyl)-3-[(3-hydroxypyrrolidin-1-yl)methyl]-1H-pyrrolo[2,3-c]pyridine-5-carboxylate. ¹H NMR (CDCl₃): [symbol] 8.72 (s, 1H), 8.54 (s, 1H), 7.10-7.20 (m, 2H), 6.99-7.05 (t, 2H), 5.37 (s, 2H), 4.34 (m, 1H), 4.01 (s, 3H), 3.48 (s, 2H), 2.89 (m, 1H), 2.68 (m, 1H), 2.60 (m, 1H), 2.39 (m, 1H), 2.19 (m, 1H), 1.77 (m, 1H). LC/MS (API-ES, M+H⁺): 384.1.

(b) 1-(4-Fluorobenzyl)-3-[(3-hydroxypyrrolidin-1-yl)methyl]-1H-pyrrolo[2,3-c]pyridine-5-carboxylic acid. ¹H NMR (DMSO-d₆): [symbol] 8.87 (s, 1H), 8.48 (s, 1H), 7.90 (s, 1H), 7.33-7.35 (m, 2H), 7.13-7.16 (t, 2H), 5.57 (s, 2H), 4.42 (m, 1H), 4.01 (s, 2H), 2.93 (m, 1H), 2.83 (m, 1H), 2.71 (m, 1H), 2.54 (m, 1H), 2.00 (m, 1H), 1.62-1.63 (m, 1H). LC/MS (API-ES, M+H⁺): 370.2.

(c) 1-(4-Fluorobenzyl)-N-hydroxy-3-[(3-hydroxypyrrolidin-1-yl)methyl]-N-methyl-1H-pyrrolo[2,3-c]pyridine-5-carboxamide. ¹H NMR (MeOH-d₄) δ: 8.74 (s, 1H), 8.32 (s, 1H), 7.72 (s, 1H), 7.27-7.31 (m, 2H), 7.04-7.10 (t, 2H), 5.55 (s, 2H), 4.33-4.38 (m, 1H), 3.88-3.99 (m, 2H), 3.43 (s; 3H), 2.86-2.91 (m, 2H), 2.55-2.65 (m, 2H), 2.11-2.22 (m, 1H), 1.70-1.74 (m, 1H). LC/MS (APCI, M+H⁺): 399.1. HRMS calcd for C₂₁H₂₄FN₄O₃ (M+H⁺) 399.1832, found 399.1842. Anal. (C₂₁H₂₃FN₄O₃.H₂O)C, H, N. HPLC: 100% purity.

### Example 19

### 1-(4-fluorobenzyl)-N-hydroxy-N-methyl-3-{[3-(methylsulfonyl)pyrrolidin-1-yl]methyl}-1H-pyrrolo[2,3-c]pyridine-5-carboxamide

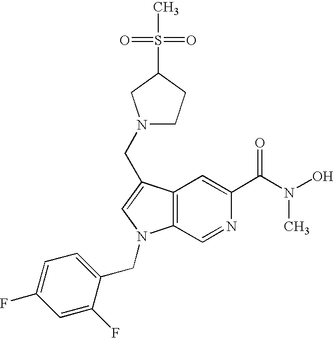

¹H NMR (300 MHz, MeOH-d₄) δ (ppm): 9.08 (s, 1H), 8.83 (b, 1H), 8.33 (s, 1H), 7.38-7.23 (m, 2H), 7.08-6.93 (m, 2H), 5.63 (s, 2H), 4.70 (s, 2H), 4.18-4.00 (m, 1H), 3.93-3.78 (m, 1H), 3.78-3.64 (m, 1H), 3.58-3.42 (m, 2H), 3.36 (s, 3H), 2.95 (s, 3H), 2.53-2.33 (m, 2H). HRMS calcd for C₂₂H₂₆FN₄O₄FS (M+H⁺) 461.1659, found 461.1666. HPLC: 98% purity.

### Example 20

### 1-(2,4-Difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide

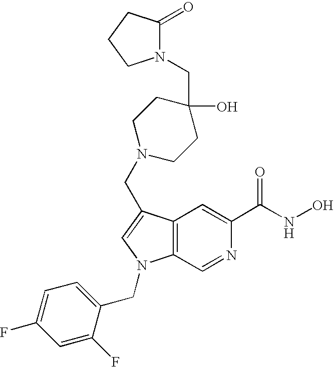

(a) tert-Butyl 1-Oxa-6-azaspiro[2,5]octane-6-carboxylate. NaH in mineral oil (10 g, 0.25 mol, 50%) was added in portions with stirring under an atmosphere of Ar to DMSO (150 mL) over a period of 20 min. Then the mixture was heated on a water-bath to 75° C. and stirred about 20 min at room temperature until evolution of hydrogen ceased. The mixture was cooled to 25° C., diluted by adding THF (150 mL), cooled to 5° C. and treated with a solution of trimethylsulfonium iodide (51 g, 0.25 mol) in DMSO (200 mL). Then compound 1 (40 g, 0.2 mol) was added to the mixture. The reaction was heated to 25° C. and stirred at this temperature for ˜2 h. Then pH was adjusted to 8 with glacial acetic acid and the mixture was diluted by adding water (500 mL), ethylacetate (400 mL), hexane (200 mL) and dichloromethane (100 mL). The organic layer was separated, washed with water (2×200 mL), diluted by adding dichloromethane (100 mL) and washed with brine. Combined aqueous layer was extracted with ethylacetate (300 mL) and hexane (150 mL). The organic layer was washed similarly. Then organic layers were filtered sequentially through SiO₂ (50 g). The combined filtrate was evaporated and the residue (45 g) was crystallized from hexane (60 mL) at −18° C. to give the title compound as white crystals (bp 56-59° C.) in 65% (28 g, 0.13 mol) yield.

(b) tert-Butyl 4-hydroxy-4-[(2-oxoimidazolidin-1-yl)methyl]piperidine-1-carboxylate.

To a solution of tert-butyl 1-Oxa-6-azaspiro[2,5]octane-6-carboxylate (45 g, 0.21 mol) and imidazolidin-2-one (17.9 g, 0.23 mol) in DMFA (200 mL) was added in portions at room temperature under an atmosphere of Ar and with stirring 60% NaH (9.3 g, 0.23 mol) in oil. The mixture was stirred at room temperature for 18 h and then additional portions of imidazolidin-2-one (4 mL) and NaH (1.8 g) were added. The mixture was heated at a water bath for 2 h. The course of the reaction was monitored by TLC (chloroform/isopropanol 20:1). The mixture was diluted by adding water (300 mL) and chloroform (200 mL). The layers were separated and the aqueous one was extracted with chloroform (3×100 mL). Combined organic layer washed with saturated NaCl and filtered through SiO₂ (25 g; 63/200 μm) and Na₂SO₄. The filtrate was evaporated. The residue was subjected to chromatography (gradient elution from carbon tetrachloride/chloroform 100:0→75:25→50:50→0:100 to chloroform/methanol 99:1→98:2→97:3→95:5) on SiO₂ (500 g; 40/63 μm). The eluate was evaporated to give the title compound in 83% (52 g, 0.17 mol) yield.

(c) 1-(4-Hydroxy-piperidin-4-ylmethyl)-pyrrolidin-2-one. tert-Butyl 4-hydroxy-4-[(2-oxoimidazolidin-1-yl)methyl]piperidine-1-carboxylate (52 g, 0.17 mol) was dissolved in chloroform (200 mL) and treated with trifluoroacetic acid (62 mL). The reaction mixture was stirred at room temperature for 18 h and then evaporated in vacuo. The residue was treated with water/ice mixture (150 mL) and chloroform (200 mL). The layers were separated and the aqueous one was extracted with chloroform (2×200 mL). The aqueous layer was made alkaline with K₂CO₃ to pH 13-14 and extracted with chloroform/isopropanol (4:1) mixture (3×200 mL). The extracts were filtered through SiO₂ (5 g; 63/200 μm) and Na₂SO₄ and evaporated. The residue was recrystallized from chloroform/ether mixture to give the title compound as yellowish crystals in ˜41% (13.8 g, 0.07 mol) yield. Satisfactory C, H, N-analysis was obtained. ¹H NMR (300 MHz, DMSO-D6) δ 4.36 (s, 1H), 3.51 (t, 2H), 3.11 (s, 2H), 2.76-2.66 (m, 2H), 2.65-2.54 (m, 2H), 2.20 (t, 2H), 1.89 (q, 2H), 1.32 (t, 4H), LC MS, API-ES: 199.3

(d) Methyl 1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxylate. The title compound was prepared from methyl 1-(2,4-difluorobenzyl)-3-formyl-1H-pyrrolo[2,3-c]pyridine-5-carboxylate and 1-[(4-hydroxypiperidin-4-yl)methyl]pyrrolidin-2-one in a manner similar to step (f) of example 25. 1H NMR (300 MHz, CHLOROFORM-D) δ ppm 1.52-1.63 (m, 4H) 1.99-2.11 (m, 2H) 2.35-2.48 (m, 4H) 2.63 (d, J=11.30 Hz, 2H) 3.26 (s, 2H) 3.50 (t, J=7.06 Hz, 2H) 3.71 (s, 2H) 3.85 (s, 1H) 4.00 (s, 3H) 5.38 (s, 2H) 6.76-6.90 (m, 2H) 7.02 (td, J=8.48, 6.22 Hz, 1H) 7.26 (s, 1H) 8.56 (d, J=0.94 Hz, 1H) 8.78 (s, 1H).

(e) 1-(2,4-Difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide. The title compound was prepared from methyl1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxylate using the methods in example 26. 1H NMR (300 MHz, MeOH) δ ppm 8.80 (s, 1H) 8.42 (s, 1H) 7.69 (s, 1H) 7.25-7.34 (m, 1H) 6.98-7.06 (m, 1H) 6.91-6.97 (m, 1H) 5.59 (s, 2H) 4.06 (s, 2H) 3.60 (t, J=7.06 Hz, 2H) 3.24-3.27 (m, 2H) 2.91 (s, 2H) 2.79 (d, J=7.54 Hz, 2H) 2.35 (t, J=8.01 Hz, 2H) 1.96-2.07 (m, 2H) 1.65 (s, 4H). LCMS (APCI, M+H⁺): 514.3. Anal. (C₂₆H₂₉F₂N₅O₄×2.0H₂O×0.07AcOH)C, H, N.

### Example 21

### 1-(2,4-difluorobenzyl)-N-hydroxy-3-{[(7R,8aS)-7-hydroxyhexahydropyrrolo[1,2-a]pyrazin-2(1H)-yl]methyl}-1H-pyrrolo[2,3-c]pyridine-5-carboxamide

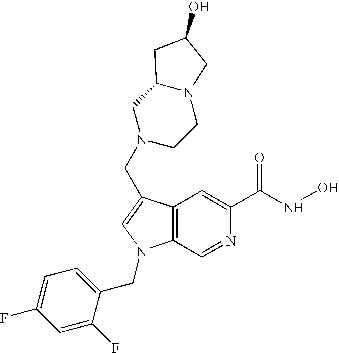

(a) Methyl (2S,4R)-4-Hydroxypyrrolidine-2-carboxylate Hydrochloride. Absolute methanol (8.0 equiv) was poured into a four-necked flask equipped with a mechanical stirrer, thermometer, reflux condenser and a drop funnel. L-hydroxyproline (1 equiv) was placed into the flask with stirring. To the suspension obtained was added dropwise at 10-15° C. distilled thionyl chloride (1.1 equiv). Then the mixture was stirred at 45° C. until TLC indicated completion (5 h) of the reaction. The suspension was cooled to 5-10° C. and then filtered and washed with dry diethyl ether. The mother liquor was evaporated in vacuo and the residue was recrystallized from dry methanol to give the title compound in 92-97% yield.

(b) 1-tert-Butyl 2-Methyl (2S,4R)-4-Hydroxypyrrolidine-1,2-dicarboxylate. Chloroform (1.4 L) and compound methyl (2S,4R)-4-hydroxypyrrolidine-2-carboxylate hydrochloride (544.8 g) were mixed under stirring. Triethylamine (464 mL, 3.3 mol) was added under cooling to the mixture and the precipitate dissolved almost completely. A solution of Boc₂O (687.5 g, 3.15 mol) in chloroform (1 L) was added dropwise at the temperature less than 25° C. for 1.5-2 h. Then the reaction mixture was heated to 45-50° C. and kept at this temperature for 2 h under stirring. The course of the reaction was monitored by TLC (chloroform/methanol 10:3). Then the reaction mixture washed sequentially with water (500 mL, 200 mL and 100 mL), a solution of citric acid (28.8 g, 0.15 mol) in water (100 mL), a solution of sodium hydroxide (12 g) in water (100 mL) and water. The solution was dried with potassium carbonate for 2-3 h and then evaporated to give a viscous light-yellow liquid. The liquid was treated with diethyl ether (400 mL), stirred and cooled for 1 h. The product washed with diethyl ether (3×300 mL) and dried to constant weight to give 615-620 g of 3 as white crystals. The mother solution of ether was evaporated and cooled to room temperature. Ether (50 mL) and a small amount of the product were added to the solution. The precipitate formed was filtered, washed with ether and dried to give 50 g of the title compound. The total yield was 96%.

(c) 1-tert-Butyl 2-Methyl (2S,4R)-4-(Benzoyloxy)pyrrolidine-1,2-dicarboxylate.

To a solution of 1-tert-butyl 2-methyl (2S,4R)-4-hydroxypyrrolidine-1,2-dicarboxylate. (73.6 g, 0.3 mol) in dichloromethane (500 mL) was added under stirring triethylamine (62.6 mL, 0.45 mol). Then, benzoylchloride (41.8 mL, 0.36 mol) in dichloromethane (100 mL) was added dropwise. The reaction mixture was stirred at room temperature for 24 h and then treated with 1 M HCl (500 mL). After 1 h, the organic layer was separated, washed sequentially with water (300 mL), 10% K₂CO₃ solution (300 mL) and water (300 mL), dried over Na₂SO₄ and evaporated to give 117 g of the title compound.

(d) Methyl (2S,4R)-4-(Benzoyloxy)pyrrolidine-2-carboxylate Hydrochloride.

To compound 1-tert-butyl 2-methyl (2S,4R)-4-(benzoyloxy)pyrrolidine-1,2-dicarboxylate (117 g) was added 4 M HCl in dioxane (300 mL), which caused intensive evolution of gas. The reaction mixture was stirred at room temperature for 3 h and evaporated. The liquid residue was dissolved in hot THF (300 mL) and left to stand in a refrigerator to give the title compound as white crystals in 95.6% (77.4 g) yield.

(e) Methyl (2S,4R)-1-(Aminoacetyl)-4-(benzoyloxy)pyrrolidine-2-carboxylate hydrochloride. To a solution of methyl (2S,4R)-4-(benzoyloxy)pyrrolidine-2-carboxylate hydrochloride. (79 g, 0.293 mol), Boc-glycine (56.4 g, 0.322 mol) and BOP (142.5 g, 0.322 mol) in dichloromethane (600 mL) was added under stirring DIPEA (113 mL, 0.644 mol). The reaction mixture was stirred at room temperature for 24 h. Then, N,N-diethylenediamine (3.5 g) was added to the mixture, which was evaporated after 1 h. The residue was dissolved in ethylacetate (500 mL) and washed with water (200 mL), 10% K₂CO₃ solution (2×200 mL), water (100 mL), saturated NaCl solution (100 mL), 1 M HCl (100 mL) and saturated NaCl solution (200 mL). The organic layer was dried over anhydrous Na₂SO₄ and evaporated to give 137 g of dipeptide, which was then treated with 4 M HCl in dioxane (300 mL). The reaction mixture was stirred at room temperature for 12 h, evaporated to constant weight and the residue washed with ether (3×150 mL) to give 117 g of the title compound.

(f) (7R,8aS)-1,4-Dioxooctahydropyrrolo[1,2-a]pyrazin-7-yl benzoate. To a solution of Methyl(2S,4R)-1-(aminoacetyl)-4-(benzoyloxy)pyrrolidine-2-carboxylate hydrochloride (117 g) in methanol (600 mL) was added triethylamine (50 mL). The reaction mixture was stirred at room temperature for 24 h and then evaporated to constant weight. The liquid residue was treated with 1 M HCl (300 mL) and chloroform (300 mL). The organic layer was separated and the aqueous one was extracted with chloroform (3×100 mL). Combined organic extracts were washed with water (300 mL) and dried over anhydrous Na₂SO₄. Then, chloroform was evaporated and the liquid residue (70 g) was treated with hot ether (200 mL). Cooling of this solution gave 59 g (73% yield) of the title compound as yellow precipitate.

(g) (7R,8aS)-Octahydropyrrolo[1,2-a]pyrazin-7-ol. To a suspension of LiAlH₄ (25.76 g, 0.678 mol) in THF (400 mL) was added under a stream of Ar with stirring and heating a suspension of (7R,8aS)-1, 4-dioxooctahydropyrrolo[1,2-a]pyrazin-7-yl benzoate (59 g, 0.2 mol) in THF (300 mL) keeping the solvent simmered. After the addition was completed, the reaction mixture was refluxed for 5 h. Then, the mixture was cooled to room temperature and quenched by the addition of 5 M aqueous NaOH (300 mL). The organic layer was separated and the curdy precipitate washed with ether (3×100 mL). Combined organic extracts were dried over anhydrous K₂CO₃ and evaporated. The liquid residue was passed through a layer of silica gel and crystallized from THF (150 mL) to give the title compound in 50.1% (14.56 g) yield. Satisfactory C, H, N-analysis was obtained. ¹H NMR (CHLOROFORM-D): [symbol] 4.42 (q, 1H), 2.50 (q, 1H), 3.09 (d, 1H), 2.94 (d, 2H), 2.81 (t, 1H), 2.42 (t, 1H), 2.26 (m, 2H), 2.12 (m, 1H), 2.02 (bs, 1H), 1.71 (dd, 1H) LC-MS (API-ES, pos.): 143.3

(h) Methyl 1-(2,4-difluorobenzyl)-3-{[(7R,8aS)-7-hydroxyhexahydropyrrolo[1,2-a]pyrazin-2(1H)-yl]methyl}-1H-pyrrolo[2,3-c]pyridine-5-carboxylate. 1H NMR (300 MHz, CHLOROFORM-D) δ ppm 1.61-1.73 (m, 4H) 1.74-1.88 (m, 1H) 2.12 (dd, J=9.70, 5.18 Hz, 1H) 2.21 (td, J=11.11, 2.83 Hz, 1H) 2.39 (td, J=10.93, 2.45 Hz, 1H) 2.47 (dd, J=6.78, 3.20 Hz, 1H) 2.83 (s, 1H) 2.88-2.97 (m, 2H) 3.47 (dd, J=9.61, 6.78 Hz, 1H) 3.69-3.79 (m, 2H) 4.01 (s, 3H) 4.46 (s, 1H) 5.38 (s, 2H) 6.77-6.90 (m, 2H) 7.04 (td, J=8.34, 6.31 Hz, 1H) 7.27 (s, 1H) 8.56 (d, J=0.94 Hz, 1H) 8.79 (s, 1H). LCMS (ESI, M+H⁺): 457.1.

(i) 1-(2,4-Difluorobenzyl)-N-hydroxy-3-{([(7R,8aS)-7-hydroxyhexahydropyrrolo[1,2-a]pyrazin-2(1H)-yl]methyl}-1H-pyrrolo[2,3-c]pyridine-5-carboxamide. 1H NMR (300 MHz, MeOH) δ ppm 8.77 (s, 1H) 8.41 (d, J=0.94 Hz, 1H) 7.60 (s, 1H) 7.28 (td, J=8.52, 6.50 Hz, 1H) 7.02 (ddd, J=10.46, 9.04, 2.54 Hz, 1H) 6.90-6.97 (m, 1H) 5.56 (s, 2H) 4.37 (d, J=1.88 Hz, 1H) 3.88 (s, 2H) 3.42 (dd, J=10.08, 6.88 Hz, 1H) 3.03 (t, J=11.77 Hz, 2H) 2.92 (d, J=11.87 Hz, 1H) 2.62-2.74 (m, 1H) 2.51 (td, J=11.26, 2.17 Hz, 1H) 2.37 (td, J=11.16, 2.92 Hz, 1H) 2.23 (dd, J=10.17, 5.09 Hz, 1H) 2.03 (t, J=10.46 Hz, 1H) 1.68-1.77 (m, 2H). LCMS (APCI, M+H⁺): 458.2. Anal. (C₂₃H₂₅F₂N₅O₃×1.7H₂O×0.08AcOH)C, H, N.

### Example 22

### 1-(2,4-Difluorobenzyl)-3-({[(1-ethylpyrrolidin-2-yl)methyl]amino}methyl)-N-hydroxy-1H -pyrrolo[2,3-c]pyridine-5-carboxamide

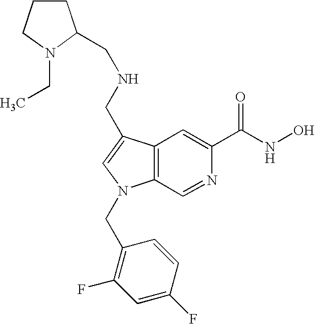

¹H NMR (CDCl₃) δ: 10.35 (bs), 8.69 (1H, s), 8.44 (1H, s), 7.66 (1H, s), 7.06 (2H, m), 6.83 (2H, m), 5.37 (2H, s), 4.37 (2H, s), 3.40-3.10 (3H, m), 3.10-2.80 (2H, s), 2.60-2.25 (2H, m), 2.15 (1H, m), 1.90-1.70 (3H, m), 1.10 (3H, t, J=7.2 Hz). LCMS (API-ES M+H⁺) 444. Anal. HPLC: >95% purity.

### Example 23

### 3-{[[2-(1-cyclopropyl-5-oxopyrrolidin-2-yl)ethyl](methyl)amino]methyl}-1-(2,4-difluorobenzyl)-N-hydroxy-1H-pyrrolo[2,3-c]pyridine-5-carboxamide

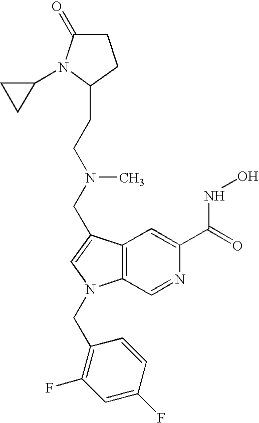

(a) 1,4-Dioxaspiro[4,5]decan-8-one Oxime (2). In a 2 L three-neck flask equipped with reflux condenser, magnetic stirrer and a thermometer, 1,4-dioxaspiro[4,5]decan-8-one (250 g, 1.6 mol) and hydroxylamine hydrochloride (167.5 g, 2.41 mol) were added to ethanol (900 mL). A solution of NaOH (90 g, 2.25 mol) in water (30 mL) was added to this mixture. The reaction mixture warmed to ˜40° C. and then heated to 50-55° C. and kept at this temperature for 1-1.5 h. The course of the reaction was monitored by TLC (chloroform/methanol 10:1; R_{f} of the starting material—0.77; R_{f} of the product—0.63). After TLC indicated completion, the reaction mixture was cooled, and inorganic precipitate was removed by filtration. The filtrate was evaporated, and water was added to the residue. The mixture was extracted with chloroform (500 mL+2×100 mL). The combined organic extracts were dried over anhydrous sodium sulfate and evaporated to give the title compound as a light-brown syrup in almost quantitative yield, which was used in the next stage without additional purification. Note: Usually yield exceeds 100% because of the residual chloroform, which facilitates dissolution in THF in the next stage.

(b) 1,4-Dioxa-8-azaspiro[4,6]undecan-9-one. In a 4 L flask equipped with a reflux condenser, magnetic stirrer, thermometer, and cooling bath, 1,4-dioxaspiro[4,5]decan-8-one Oxime (384 g, 2.25 mol) was dissolved in THF (1.3 L). A solution of NaOH (234 g, 5.85 mol) in water (1.75 L) was added in one portion to the solution. Then, benzenesulfonyl chloride (287 mL, 2.25 mol) was added dropwise over a period of 2-3 h. The reaction mixture turned dark and warmed up. The temperature of the reaction mixture was kept below 55-60° C., so that the mixture did not boil. The reaction mixture was stirred overnight, then it was evaporated, and the brown precipitate was filtered and washed 3 times with chloroform (200-300 mL). Water (700 mL) was added to the mother liquor, the organic layer was separated, and the aqueous layer was extracted with chloroform (500 mL+3×200 mL). The combined organic layers were washed with water (100 mL), dried over anhydrous sodium sulfate, and evaporated. Cold diethyl ether (100 mL) was added to the resulting syrup, and precipitate was separated by filtration, washed 2-3 times with minimal amount of cold ether, and air-dried to give 227 g (59%) of the title compound as a snow-white powder. Note: Additional amount of the product can be recovered form the mother solution after precipitation and washing of the precipitate with ether.

(c) 8-Methyl-1,4-dioxa-8-azaspiro[4,6]undecan-9-one

1,4-Dioxa-8-azaspiro[4,6]undecan-9-one (75.0 g, 0.438 mol) was added in portions to a vigorously stirred suspension of NaH (16.56 g, 0.69 mol), which was preliminary washed with hexane three times to eliminate mineral oil, in anhydrous THF (1.2 L). The mixture was cooled to 0-5° C. under an atmosphere of Argon. Evolution of hydrogen was observed over a period of 10-15 min after addition of the last portion of the template. Then 18-crown-6 (1.3 g) was added. The mixture was stirred for 5-10 min, and iodomethane (93.25 g, 41 mL, 0.657 mol) was added quickly into the suspension. The mixture obtained was stirred at 30-35° C. for 2 h and then allowed to stand until TLC (silica gel 60; chloroform/methanol 15:1) indicated completion (overnight) of the reaction. Then, methanol (30 mL) was added dropwise to quench excessive sodium hydride. The solvent was removed under reduced pressure and the residue was diluted by adding chloroform (500 mL). The suspension obtained was filtered through a column (17 cm in diameter), containing silica gel (7 cm layer). Silica gel was washed with chloroform (5×150 mL) and combined filtrates were evaporated under reduced pressure to give the title compound as yellow oil in 92% (149.27 g) yield.

(d) 1-Methylazepane-2,5-dione

8-Methyl-1,4-dioxa-8-azaspiro[4,6]undecan-9-one (4) (150.3 g, 0.811 mol) was dissolved in concentrated HCOOH (d 1.22, 310 mL) under stirring and slight heating. This solution was stirred at 40-45° C. for 10 h. Then, the mixture was cooled on an ice bath, neutralized to pH 9 using several small portions of solid K₂CO₃, diluted by adding chloroform (500 mL) and allowed to stand overnight. The course of the reaction was monitored by TLC (silica gel 60; chloroform/methanol 15:1). Then, the mixture was filtered through a layer of silica gel (17 cm in diameter and 5 cm thick). The product was eluted with chloroform (˜3 L). The eluate was concentrated under reduced pressure to give 82.04 g (72% yield) of the title compound as pale-yellow crystals. ¹H NMR spectrum is attached.

(e) 1-Cyclopropyl-5-(2-methylamino-ethyl)-pyrrolidin-2-one (6)

Cyclopropanamine (11.43 g, 0.2 mol) was added under vigorous stirring to a suspension of NaBH(OAc)₃ (55.0 g, 0.26 mol) in anhydrous dichloromethane (400 mL). Note: foaming was observed during this operation. Then, to this mixture cooled in an ice bath was added a solution of 1-methylazepane-2,5-dione (28.23 g, 0.2 mol) in dichloromethane (200 mL). The reaction mixture was stirred for 3 h at room temperature and then allowed to stand overnight. The course of the reaction was monitored by TLC (silica gel 60; chloroform/methanol 10:1). The reaction mixture was diluted by adding water (200 mL) to decompose excessive NaBH(OAc)₃. The emulsion obtained was stirred for 30 min at room temperature, and the layers were separated. The aqueous layer washed with chloroform (3×50 mL) and then treated with solid K₂CO₃ to pH 7. The mixture obtained was extracted again with chloroform (10×50 mL) to remove impurities. The aqueous solution was then treated with solid K₂CO₃ to obtain pH 8, extracted with chloroform (2×50 mL) and then additionally treated with solid K₂CO₃ to obtain pH 10 and finally extracted with chloroform (5×50 mL). The extracts were combined and concentrated under reduced pressure to give the title compound as almost colorless oil in 53% (19.34 g) yield. Satisfactory C, H, N-analysis was obtained. ¹H NMR (CHLOROFORM-D) δ 3.49-3.63 (m, 1H) 2.56-271 (m, 2H) 2.47 (s, 3H) 2.38-2.44 (m, 2H) 2.15-2.34 (m, 1H) 2.00-2.15 (m, 2H) 1.50-1.78 (m, 3H) 0.92-1.02 (m, 1H) 0.75-0.86 (m, 1H), 0.63-0.71 (m, 1H) 0.50-0.59 (m, 1H); LCMS (API-ES, pos.): 183.3

(f) Methyl 3-{[[2-(1-cyclopropyl-5-oxopyrrolidin-2-yl)ethyl](methyl)amino]methyl}-1-(2,4-difluorobenzyl)-1H-pyrrolo[2,3-c]pyridine-5-carboxylate: ¹H NMR (CD₃OD) δ: 8.84 (s, 1H), 8.56 (s, 1H), 7.64 (s, 1H), 7.32-7.35 (m, 1H), 6.95-7.04 (m, 2H), 5.59 (s, 2H), 3.95 (s, 3H), 3.78 (q, 2H), 3.58-3.59 (m, 1H), 2.48-2.53 (m, 1H), 2.20-2.41 (m, 3H), 2.10-2.20 (s, 2H), 1.85-1.95 (m, 1H), 1.58-1.64 (m, 2H), 0.85-0.88 (m, 1H), 0.50-0.70 (m, 3H). LC/MS (APCI, M+H⁺): 497.2.

(g) 3-{[[2-(1-cyclopropyl-5-oxopyrrolidin-2-yl)ethyl](methyl)amino]methyl}-1-(2,4-difluorobenzyl)-N-hydroxy-1 pyrrolo[2,3-c]pyridine-5-carboxamide. ¹H NMR (DMSO-d₆) δ: 10.98-11.10 (bs, 1H), 8.81 (s, 1H), 8.28 (s, 1H), 7.65 (s, 1H), 7.27-7.36 (m, 2H), 7.07 (t, 1H), 5.59 (s, 2H), 3.65 (q, 2H), 3.41-3.46 (m, 1H), 2.30-2.40 (m, 1H), 2.10-2.30 (m, 3H), 1.90-2.05 (m, 2H), 1.65-1.75 (m, 1H), 1.30-1.50 (m, 2H), 0.65-0.75 (m, 1H), 0.40-0.55 (m, 3H). LC/MS (APCI, M+H⁺): 498.2. Anal. (C₂₆H₂₉F₂N₅O₃.0.8H₂O)C, H, N. HPLC: 98.5% purity.

### Example 24

### 1-(2,4-difluorobenzyl)-N-hydroxy-3-({[1-(hydroxymethyl)cyclopentyl]amino}methyl)-1H-pyrrolo[2,3-c]pyridine-5-carboxamide

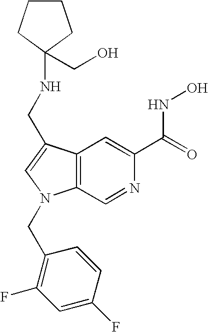

(a) Methyl 1-(2,4-difluorobenzyl)-3-({[1-(hydroxymethyl)cyclopentyl]amino}methyl)-1H-pyrrolo[2,3-c]pyridine-5-carboxylate: The title compound was prepared from methyl 1-(2,4-difluorobenzyl)-3-formyl-1H-pyrrolo[2,3-c]pyridine-5-carboxylate and 1-(hydroxymethyl)cyclopentyl]amine in a manner similar to step (f) of example 25. ¹H NMR (MeOD-d₄) δ 8.90 (s, 1H), 8.83 (s, 1H), 8.58 (s, 1H), 7.66 (s, 1H), 7.31-7.34 (m, 1H), 6.93-7.04 (m, 2H), 5.57 (s, 2H), 3.96 (s, 3H), 3.93 (s, 2H), 3.58 (s, 2H), 1.59-1.78 (m, 8H). LC/MS (APCI, M+H⁺): 430.2.

(b) 1-(2,4-difluorobenzyl)-N-hydroxy-3-({[1-(hydroxymethyl)cyclopentyl]amino}methyl)-1H-pyrrolo[2,3-c]pyridine-5-carboxamide. The title compound was prepared from methyl 1-(2,4-difluorobenzyl)-3-({[1-(hydroxymethyl)cyclopentyl]amino}methyl)-1H-pyrrolo[2,3-c]pyridine-5-carboxylate using the methods in example 26. ¹H NMR (DMSO-d₆) δ: 11.10 (s, 1H), 8.90 (s, 1H), 8.80 (s, 1H), 8.26 (s, 1H), 7.61 (s, 1H), 7.29-7.35 (m, 2H), 7.07 (t, 1H, J=7.7 Hz), 5.57 (s, 2H), 4.57 (m, 1H), 3.79 (s, 2H), 3.38 (d, 2H, J=4.3 Hz), 1.66-1.70 (m, 2H), 1.40-1.60 (m, 6H). LC/MS (APCI, M+H⁺): 431.2. Anal. (C₂₂H₂₄F₂N₄O₃.0.2H₂O)C, H, N. HPLC: 100% purity.

### Example 25

### 1-(2,4-difluorobenzyl)-N-hydroxy-3-({[1-(hydroxymethyl)cyclopentyl]amino}methyl)-N-methyl-1H-pyrrolo[2,3-c]pyridine-5-carboxamide

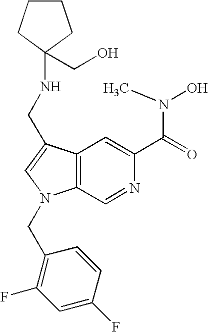

(a) 1-(2,4-difluorobenzyl)-3-({[1-(hydroxymethyl)cyclopentyl]amino}methyl)-1H-pyrrolo[2,3-c]pyridine-5-carboxylic acid. ¹H NMR (DMSO-d₆): [symbol] : 8.94 (s, 1H), 8.50 (s, 1H), 7.82 (s, 1H), 7.32-7.74 (m, 2H), 7.09 (t, 1H, J=8.5 Hz), 5.63 (s, 2H), 4.09 (s, 2H), 3.50 (s, 2H), 1.60-1.75 (m, 6H), 1.50-1.60 (m, 2H). LC/MS (API-ES, M+H⁺): 416.2.

(b) 1-(2,4-difluorobenzyl)-N-hydroxy-3-({[1-(hydroxymethyl)cyclopentyl]amino}methyl)-N-methyl-1H -pyrrolo[2,3-c]pyridine-5-carboxamide. ¹H NMR (MeOH-d₄) δ: 8.90 (s, 1H), 8.31 (s, 1H), 7.84 (s, 1H), 7.40 (q, 1H, J=6.4 Hz), 6.96-7.05 (m, 2H), 5.61 (s, 2H), 4.34 (s, 2H), 3.70 (s, 2H), 3.43 (s, 3H), 1.65-1.91 (m, 8H). LC/MS (APCI, M+H⁺): 445.2. Anal. (C₂₃H₂₆F₂N₄O₃.0.8H₂O.CH₃COOH)C, H, N. HPLC: 100% purity.

### Example 26

### 1-(2,4-difluorobenzyl)-3-({[1-(hydroxymethyl)cyclopentyl]amino}methyl)-N-methoxy-1H-pyrrolo[2,3-c]pyridine-5-carboxamide

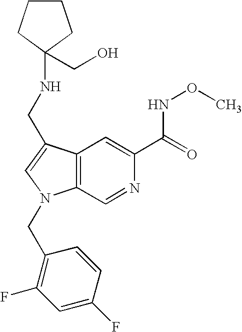

¹H NMR (MeOH-d₄) δ: 8.88 (s, 1H), 8.48 (s, 1H), 7.77 (s, 1H), 7.37 (q, 1H, J=6.2 Hz), 6.90-7.04 (m, 2H), 5.61 (s, 2H), 4.34 (s, 2H), 3.83 (s, 3H), 3.71 (s, 2H), 1.65-1.91 (m, 8H). LC/MS (APCI, M+H⁺): 445.2. Anal. (C₂₃H₂₆F₂N₄O₃.0.8H₂O.CH₃COOH)C, H, N. HPLC: 95.0% purity.

### Example 27

### 3-{[[2-(1-cyclopropyl-5-oxopyrrolidin-2-yl)ethyl](methyl)amino]methyl})-1-(2,4-difluorobenzyl)-N-hydroxy-N-methyl-1H-pyrrolo[2,3-c]pyridine-5-carboxamide

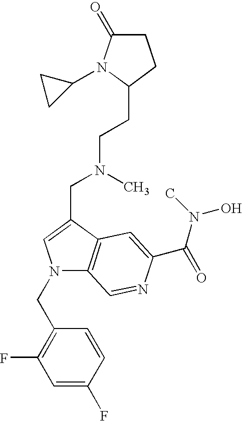

(a) 3-{[[2-(1-cyclopropyl-5-oxopyrrolidin-2-yl)ethyl](methyl)amino]methyl}-1-(2,4-difluorobenzyl)-1H-pyrrolo[2,3-c]pyridine-5-carboxylic acid. ¹H NMR (DMSO-d₆): [symbol] : 8.91 (s, 1H), 8.40 (s, 1H), 7.69 (s, 1H), 7.09-7.36 (m, 2H), 7.06 (t, 1H, J=6.70 Hz), 5.61 (s, 2H), 3.68 (q, 2H, J=8.0 Hz), 3.42-3.45 (m, 1H), 2.10-2.30 (m, 4H), 2.17 (s, 3H), 1.95-2.05 (m, 2H), 1.70-1.80 (m, 1H), 1.40-1.50 (m, 2H), 0.65-0.75 (m, 1H), 0.43-0.54 (m, 3H). LC/MS (APCI, M+H⁺): 483.2.

(b) 3-{[[2-(1-cyclopropyl-5-oxopyrrolidin-2-yl)ethyl](methyl)amino]methyl})-1-(2,4-difluorobenzyl)-N-hydroxy-N-methyl-1H-pyrrolo[2,3-c]pyridine-5-carboxamide. ¹H NMR (MeOH-d₄) [symbol] : 8.82 (s, 1H), 8.31 (s, 1H), 7.68 (s, 1H), 7.30-7.36 (m, 1H), 6.90-7.06 (m, 2H), 5.59 (s, 2H), 3.85 (q, 2H, J=10.7 Hz), 3.59-3.62 (m, 1H), 3.42 (s, 3H), 2.50-2.60 (m, 1H), 2.40-2.50 (m, 1H), 2.33 (s, 3H), 2.20-2.40 (m, 4H), 1.60-1.70 (m, 1H), 1.50-1.60 (m, 2H), 0.65-0.80 (m, 1H), 0.50-0.60 (m, 3H). LC/MS (APCI, M+H⁺): 512.3. Anal. (C₂₇H₃₁F₂N₅O₃.0.5H₂O.CH₃COOH)C, H, N. HPLC: 97.0% purity.

### Example 28

### 3-{[[2-(1-cyclopropyl-5-oxopyrrolidin-2-yl)ethyl](methyl)amino]methyl})-1-(2,4-difluorobenzyl)-N-methoxy-1H-pyrrolo[2,3-c]pyridine-5-carboxamide

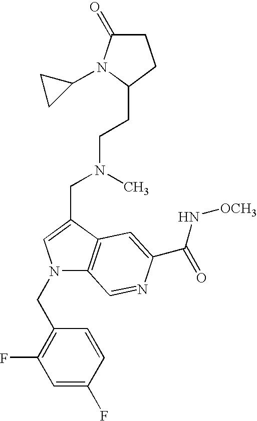

¹H NMR (MeOH-d₄) [symbol] : 8.81 (s, 1H), 8.45 (s, 1H), 7.67 (s, 1H), 7.32 (q, 1H, J=7.0 Hz), 6.90-7.06 (m, 2H), 5.59 (s, 2H), 4.00 (q, 2H, J=13.6 Hz), 3.82 (s, 3H), 3.52-3.62 (m, 1H), 2.71-2.74 (m, 1H), 2.60-2.65 (m, 1H), 3.44 (s, 3H), 2.13-2.32 (m, 4H), 1.87-1.95 (m, 1H), 1.55-1.75 (m, 2H), 0.83-0.92 (m, 1H), 0.56-0.66 (m, 3H). LC/MS (APCI, M+H⁺): 512.2. Anal. (C₂₇H₃₁F₂N₅O₃.1.5H₂O.CH₃COOH)C, H, N. HPLC: 100% purity.

### Example 29

### 3-{[3-(aminocarbonyl)piperidin-1-yl]methyl}-1-(4-fluorobenzyl)-N-hydroxy-N-methyl-1H-pyrrolo[2,3-c]pyridine-5-carboxamide

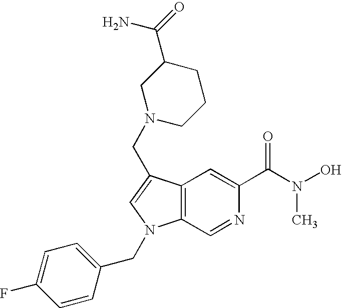

(a) Methyl 3-{[3-(aminocarbonyl)piperidin-1-yl]methyl}-1-(4-fluorobenzyl)-1H-pyrrolo[2,3-c]pyridine-5-carboxylate. ¹H NMR (MeOH-d₄) δ: 8.75 (s, 1H), 8.55 (s, 1H), 7.69 (s, 1H), 7.29-7.26 (m, 2H), 7.08-7.03 (m, 2H), 5.55 (s, 2H), 3.96 (s, 3H), 3.82 (s, 2H), 3.34 (s, 2H), 2.92-2.88 (m, 2H), 2.49-2.47 (m, 1H), 2.31-2.24 (m, 2H), 1.75-1.73 (m, 2H). LCMS (APCI, M+H⁺): 425.

(b) 3-{[3-(Aminocarbonyl)piperidin-1-yl]methyl}-1-(4-fluorobenzyl)-1H-pyrrolo[2,3-c]pyridine-5-carboxylic acid. ¹H NMR (MeOH-d₄) δ: 8.79 (s, 1H), 8.56 (s, 1H), 7.97 (s, 1H), 7.31-7.28 (m, 2H), 7.09-7.05 (m, 2H), 5.60 (s, 2H), 4.07 (s, 2H), 3.10-2.98 (m, 2H), 2.65-2.53 (m, 3H), 1.91-1.78 (m, 2H), 1.76-1.45 (m, 2H). LCMS (APCI, M+H⁺): 411.15. HPLC: 97% purity.

(c) 3-{[3-(Aminocarbonyl)piperidin-1-yl]methyl}-1-(4-fluorobenzyl)-N-hydroxy-N-methyl-1H-pyrrolo[2,3-c]pyridine-5-carboxamide. ¹H NMR (MeOH-d₄) δ: 8.76 (s, 1H), 8.31 (b, 1H), 7.78 (s, 1H), 7.31-7.28 (m, 2H), 7.09-7.05 (m, 2H), 5.56 (s, 2H), 4.02 (s, 2H), 3.43 (s, 3H), 3.17-2.93 (m, 2H), 2.56 (b, 3H), 1.89-1.83 (m, 2H), 1.78-1.45 (m, 2H). LCMS (APCI, M+H⁺): 440.10. HPLC: 97% purity.

### Example 30

### 1-(2,4-difluorobenzyl)-3-({[(1-ethylpyrrolidin-2-yl)methyl]amino}methyl)-N-methoxy-1H -pyrrolo[2,3-c]pyridine-5-carboxamide

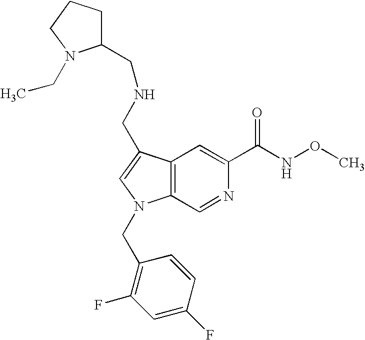

¹H NMR (MeOH-d4) δ: 8.84 (1H, s), 8.49 (1H, s), 7.68 (1H, s), 7.36 (1H, m), 7.10-6.90 (2H, m), 5.60 (2H, s), 4.15 (2H, s), 3.87 (3H, s), 3.53 (1H, m), 3.45-3.30 (2H, m), 3.20 (1H, m), 2.97 (2H, q), 2.91 (2H, m), 2.20 (1H, m), 1.99 (1H, m), 1.94 (3H, s), 1.81 (1H, m), 1.20 (3H, t). LCMS (API-ES M+H⁺) 458.20. Anal. HPLC: >95% purity. Anal. (C₂₄H₂₉F₂N₅O₂×1.54H₂O×3.00HCl) C, H, N.

### Example 32

### 1-(2,4-difluorobenzyl)-N-ethyl-N-hydroxy-3-({4-hydroxy-4-[(2-oxopyrrolidin-1-yl)methyl]piperidin-1-yl}methyl)-1H-pyrrolo[2,3-c]pyridine-5-carboxamide

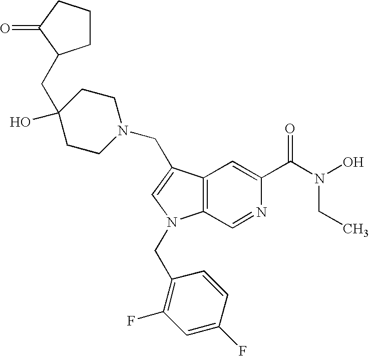

To 1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxylic acid (0.4 g, 0.8 mmol) in DMF (10 mL) were added HATU (0.669 g, 1.76 mmol), triethylamine (0.446 ml, 3.2 mmol), and N-ethylhydroxylamine hydrochloride [prep. acc. Baillie, L. C.; Batsanov, A. Bearder, J. R.; Whiting, D. J. Chem. Soc. Perkin Trans. 1; 1998, 20, 3471-3478] (0.270 g, 1.76 mmol). The resulting mixture was stirred for 18 hours at ambient temperature. The solvent was evaporated and the residue was dissolved in methanol and purified by preparative HPLC to provide the title compound as a white powder (0.237 g, 55% yield). 1H NMR (300 MHz, MeOH) 5 ppm 8.88 (s, 1H) 8.31 (s, 1H) 7.88 (s, 1H) 7.35-7.44 (m, 1H) 6.94-7.07 (m, 2H) 5.64 (s, 2H) 4.34 (s, 2H) 3.87-3.97 (m, 2H) 3.58-3.69 (m, 4H) 3.12-3.22 (m, 2H) 3.02-3.12 (m, 2H) 2.33-2.40 (m, 2H) 1.95-2.07 (m, 4H) 1.68-1.79 (m, 5H); LC-MS (APCI, M+H⁺): 542.3. HPLC: 96% purity.

### Example 33

### 1-(2,4-difluorobenzyl)-N-hydroxy-3-({4-hydroxy-4-[(2-oxopyrrolidin-1-yl)methyl]piperidin-1-yl}methyl)-N-propyl-1H-pyrrolo[2,3-c]pyridine-5-carboxamide

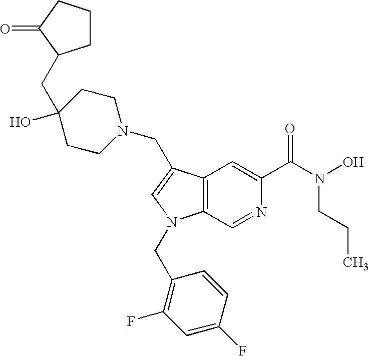

To 1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxylic acid (0.3 g, 0.6 mmol) in DMF (7 mL) were added HATU (0.342 g, 0.9 mmol), triethylamine (0.418 ml, 3 mmol), and N-propylhydroxylamine hydrochloride [prep. acc. Mellor, Sarah L.; Chan, Weng C. Chem. Commun.; 1997, 20, 2005-2006] (0.117 g, 0.9 mmol). The resulting mixture was stirred for 5 hours at ambient temperature. The solvent was evaporated and the residue was dissolved in methanol and purified by preparative HPLC to provide the title compound as a white powder (0.178 g, 53% yield). 1H NMR (300 MHz, DMSO-D6) 8 ppm 9.01 (s, 1H) 8.34 (s, 1H) 7.99 (s, 1H) 7.33-7.41 (m, 2H) 7.10-7.19 (m, 1H) 5.72 (s, 2H) 5.00 (s, 1H) 4.55-4.66 (m, 2H) 3.64-3.78 (m, 2H) 3.46-3.58 (m, 2H) 3.10-3.24 (m, 6H) 2.20-2.33 (m, 2H). 1.86-2.00 (m, 2H) 1.62-1.77 (m, 6H) 0.83-0.99 (m, 3H) LC-MS (APCI, M+H⁺): 556.3. HPLC: 96% purity.

### Example 34

### N-benzyl-1-(2,4-difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide

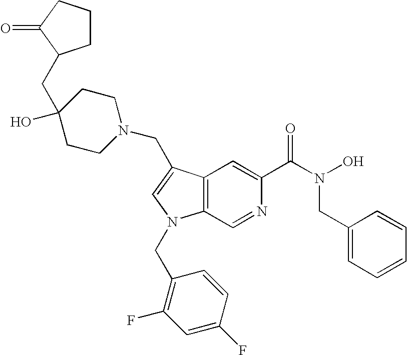

The title compound was purified by preparative HPLC to provide the title compound as a white powder (0.95 g, 26% yield). 1H NMR (300 MHz, DMSO-D6) δ ppm 9.03 (s, 1H) 8.44 (s, 1H) 7.99 (s, 1H) 7.31-7.45 (m, 8H) 7.09-7.19 (m, 1H) 5.72 (s, 2H) 5.01 (s, 3H) 4.61 (s, 2H) 3.51 (d, 2H) 3.34 (s, 2H) 3.10-3.25 (m, 4H) 2.25 (s, 2H) 1.95 (s, 2H) 1.59-1.74 (m, 4H). LC-MS (APCI, M+H⁺): 604.2. HPLC: 96% purity.

### Example 35

### 1-(2,4-difluorobenzyl)-N-hydroxy-3-({4-hydroxy-4-[(2-oxopyrrolidin-1-yl)methyl]piperidin-1-yl}methyl)-N-methyl-1H-pyrrolo[2,3-c]pyridine-5-carboxamide

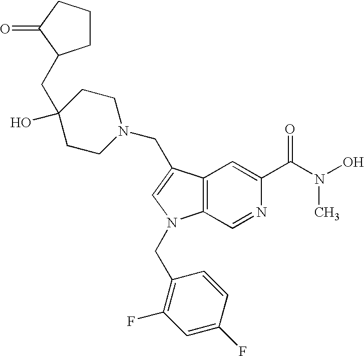

The title compound was purified by preparative HPLC to provide the title compound as a white powder (0.179 g, 56% yield). 1H NMR (300 MHz, MeOH) δ ppm 8.86 (s, 1H) 8.31 (s, 1H) 7.80 (s, 1H) 7.31-7.41 (m, 1H) 6.93-7.07 (m, 2H) 5.62 (s, 2H) 4.13 (s, 2H) 3.57-3.65 (m, 2H) 3.44 (s, 3H) 3.25-3.28 (m, 2H) 2.92-3.04 (m, 2H) 2.78-2.90 (m, 2H) 2.36 (t, 2H) 1.97-2.08 (m, 2H) 1.62-1.77 (m, 4H) LC-MS (APCI, M+H⁺): 528.3. HPLC: 96% purity.

### Example 36

### 1-(2,4-difluorobenzyl)-N-hydroxy-3-({4-hydroxy-4-[(2-oxopyrrolidin-1-yl)methyl]piperidin-1-yl}methyl)-N-(3-hydroxypropyl)-1H-pyrrolo[2,3-c]pyridine-5-carboxamide

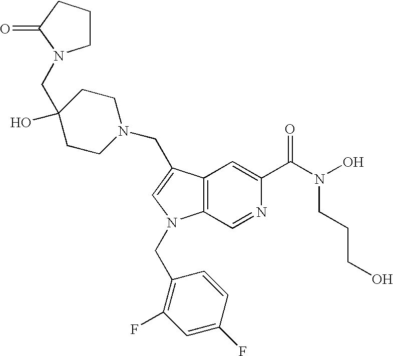

The title compound was purified by preparative HPLC to provide the title compound as a white powder (0.025 g, 7.3% yield). 1H NMR (300 MHz, MeOH) 5 ppm 8.84 (s, 1H) 8.30 (s, 1H) 7.77 (s, 1H) 7.28-7.40 (m, 1H) 6.92-7.07 (m, 2H) 5.61 (s, 2H) 4.07 (s, 2H) 3.85-3.97 (m, 2H) 3.57-3.70 (m, 4H) 3.26 (s, 2H) 2.87-2.98 (m, 2H) 2.72-2.84 (m, 2H) 2.31-2.40 (m, 2H) 1.94-2.07 (m, 4H) 1.60-1.76 (m, 4H). LC-MS (APCI, M+H⁺): 573.3. HPLC: 96% purity.

### Example 37

### 1-(2,4-difluorobenzyl)-N-ethoxy-3-({4-hydroxy-4-[(2-oxopyrrolidin-1-yl)methyl]piperidin-1-yl}methyl)-1H-pyrrolo[2,3-c]pyridine-5-carboxamide

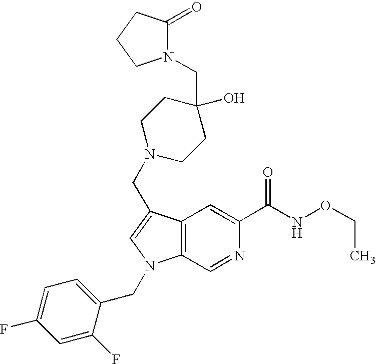

1-(2,4-Difluorobenzyl)-N-ethoxy-3-({4-hydroxy-4-[(2-oxopyrrolidin-1-yl)methyl]piperidin-1-yl}methyl)-1H -pyrrolo[2,3-c]pyridine-5-carboxamide. To 1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxylic acid (0.2 g, 0.4 mmol) in DMF (10 mL) were added HATU (0.15 g, 0.4 mmol), triethylamine (0.234 ml, 1.68 mmol), and O-ethylhydroxylamine hydrochloride (0.117 g, 1.2 mmol). The resulting mixture was stirred for 16 hours at ambient temperature. The solvent was evaporated and the residue washed by saturated sodium bicarbonate (30 mL) and dichloromethane (3×30 mL). The combined organic extracts were dried over sodium sulfate, concentrated in vacuo and purified by preparative HPLC to provide the title compound as a white powder (0.09 g, 42% yield). ¹H NMR (MeOH-d₄) δ: 8.68 (s, 1H), 8.33 (s, 1H), 7.51 (s, 1H), 7.18-7.16 (m, 1H), 6.93-6.85 (m, 2H), 5.48 (s, 2H), 3.99-3.92 (qt, 2H), 3.71 (s, 2H), 3.54-3.49 (t, 2H), 3.15 (s, 2H), 2.60-2.58 (m, 2H), 2.42-2.39 (m, 2H), 2.29-2.24 (t, 2H), 1.95-1.90 (m, 2H), 1.59-1.46 (m, 4H), 1.26-1.21 (t, 3H). LC-MS (APCI, M+H⁺): 542.20. HPLC: 96% purity.

### Example 38

### N-(benzyloxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide

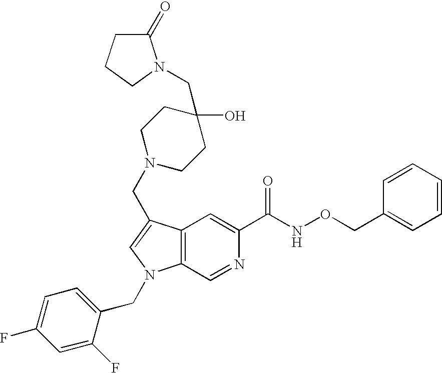

¹H NMR (MeOH-d₄) δ: 8.74 (s, 1H), 8.37 (s, 1H), 7.71 (s, 1H), 7.41-7.38 (m, 2H), 7.27-7.23 (m, 4H), 7.00-6.87 (m, 2H), 5.51 (s, 2H), 4.91 (s, 2H), 4.24 (s, 2H), 3.53-3.48 (t, 2H), 3.18 (s, 2H), 3.05-2.95 (m, 2H), 2.94-2.80 (m, 2H), 2.30-2.25 (t, 2H), 1.96-1.91 (m, 2H), 1.70-1.55 (m, 4H). LC-MS (APCI, M+H⁺): 604.30. HPLC: 99% purity.

### Example 39

### N-(cyclopropylmethoxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide

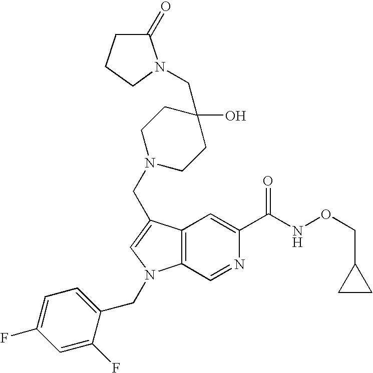

¹H NMR (MeOH-d₄) δ: 8.84 (s, 1H), 8.47 (s, 1H), 7.76 (s, 1H), 7.37-7.35 (m, 1H), 7.07-7.00 (m, 2H), 5.65 (s, 2H), 4.19 (s, 2H), 3.86-3.84 (d, 2H), 3.67-3.63 (t, 2H), 3.33 (s, 2H), 3.10-3.02 (m, 2H), 3.00-2.85 (m, 2H), 2.43-2.37 (t, 2H), 2.09-2.04 (m, 2H), 1.80-1.65 (m, 4H), 1.45-1.30 (m, 1H), 0.65-0.61 (m, 2H), 0.38-0.36 (m, 2H). LC-MS (APCI, M+H⁺): 568.20. HPLC: 98% purity.

### Example 40

### 1-(2,4-difluorobenzyl)-3-({4-hydroxy-4-[(2-oxopyrrolidin-1-yl)methyl]piperidin-1-yl}methyl)-N-phenoxy-1H-pyrrolo[2,3-c]pyridine-5-carboxamide

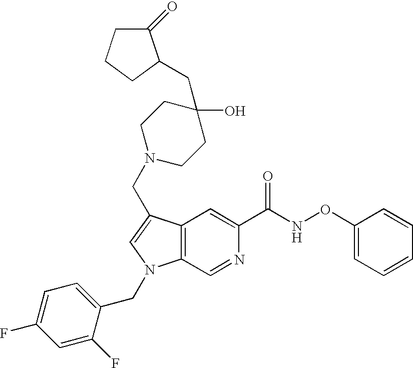

¹H NMR (MeOH-d₄) δ: 8.90 (s, 1H), 8.50 (s, 1H), 7.90 (s, 1H), 7.31-7.30 (m, 1H), 7.19-7.17 (m, 2H), 7.03-7.01 (m, 2H), 6.92-6.90 (m, 2H), 6.87-6.85 (m, 1H), 5.57 (s, 2H), 4.48 (s, 2H), 3.49-3.47 (m, 2H), 3.27-3.25 (m, 2H), 3.21-3.18 (m, 2H), 3.14 (s, 2H), 2.25-2.21 (t, 2H), 1.91-1.88 (m, 2H), 1.66-1.61 (b, 4H), LC-MS (APCI, M+H⁺): 591.05. HPLC: 91% purity.

### Example 41

### 1-(4-fluorobenzyl)-4-hydroxy-N-methoxy-1H-pyrrolo[2,3-c]pyridine-5-carboxamide

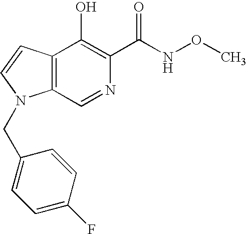

To methyl 1-(4-fluorobenzyl)-4-hydroxy-1H-pyrrolo[2,3-c]pyridine-5-carboxylate (0.6 g, 2 mmol) in methanol (10 ml) and water (1 ml) were added sodium hydroxide (0.56 g, 14 mmol) and o-methylhydroxylamine (0.668 g, 8 mmol). The resulting mixture was stirred for 28 hours at 63° C. 2 ml acetic acid was added to precipitate undesired byproduct, 1-(4-fluorobenzyl)-4-hydroxy-1H-pyrrolo[2,3-c]pyridine-5-carboxylic acid, which was removed by filtration. After removing solvent, the residue was dissolved in methanol and purified by preparative HPLC to provide the title compound as a white powder (0.035 g, 5.6% yield). 1H NMR (300 MHz, DMSO-D6) d ppm 3.76 (s, 3H) 5.60 (s, 2H) 6.75 (d, J=2.83 Hz, 1H) 7.17-7.24 (m, 2H) 7.32-7.39 (m, J=5.46 Hz, 2H) 7.76 (d, J=3.20 Hz, 1H) 8.47 (s, 1H) 12.08 (s, 1H) 12.89 (s, 1H). LC-MS (APCI, M+H⁺): 316.1. HPLC: 95% purity.

### Example 42

### 3-{[(2S)-2-(aminocarbonyl)pyrrolidin-1-yl]methyl}-1-(4-fluorobenzyl)-N-hydroxy-N-methyl-1H-pyrrolo[2,3-c]pyridine-5-carboxamide

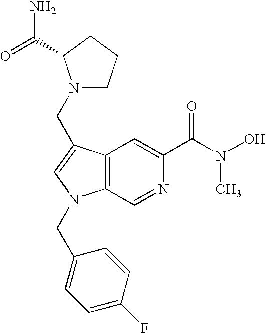

(a) Methyl 4-methyl-5-nitropyridine-2-carboxylate. HCl gas was bubbled into the solution of 2-cyano-4-methyl-5-nitropyridine (30 g) in methanol (200 mL) with cooling in a ice-water bath for 5 minutes. Then 3.3 mL water (1 eq.) was added to the flask. The resulting solution was heated to reflux for 3 hrs. The desired product precipitated as the HCl salt (white crystals). The mixture was cooled to room temperature and the precipitate was collected by vacuum filtration. The solid was transferred to a 1 L separation funnel, neutralized with saturated aqueous NaHCO₃ (400 mL), and extracted with dichloromethane (400 mL). The organic layer was dried over sodium sulfate, concentrated and dried under vacuum to give the title compound as a white solid (33 g, 92% yield). ¹H NMR (DMSO-D₆) δ, ppm: 9.19 (s, 1H), 8.21 (s, 1H), 3.91 (s, 3H), 2.63 (s, 3H). LCMS (APCI, M+H⁺): 197.0.

(b) Methyl 4-[(E)-2-(dimethylamino)vinyl]-5-nitropyridine-2-carboxylate. A mixture of methyl 4-methyl-5-nitropyridine-2-carboxylate (3.5 g, 17.8 mmol), dimethylformamide dimethylacetal (DMF-DMA) (3.6 ml, 1.5 eq) in acetonitrile (35 mL) was heated in a microwave at 140° C. for 20 min. The solvent was removed. The residue (5.1 g) was carried onto the next step without further purification. Method 2. A mixture of compound methyl 4-methyl-5-nitropyridine-2-carboxylate (39.5 g, 0.19 mol), DMF-DMA (30.6 g, 0.26 mol, 1.35 eq) in DMF (470 mL) was heated to 90° C. for 30 min. The solvent was removed in vacuo. The residue (78 g) was used without further purification in the next step.

(c) Methyl 1H-pyrrolo[2,3-c]pyridine-5-carboxylate. To a 500 mL Parr bottle was added methyl 4-[(E)-2-(dimethylamino)vinyl]-5-nitropyridine-2-carboxylate (18.9 g, 75.2 mmol) and anhydrous methanol (200 mL). The mixture was purged with nitrogen gas for 10 min. To this suspension was added Pd(10%)/C (1.90 g, 10 w/w %), and the suspension was degassed for 5 more minutes. The hydrogenation began with 43 psi H₂ without heating. The reaction became exothermic as indicated by the rising temperature (about 2-3° C. per min) inside the Parr bottle (monitored by a thermal coupling thermometer). As the temperature inside of the reaction reached 45° C., the hydrogen gas flow into the Parr bottle was stopped, the temperature was allowed to cool down to 25° C. for 30 min. The color of the liquid of the suspension changed from purple red to light green and then colorless in the first hour of the reduction, and about 30 psi H₂ was consumed. The hydrogen pressure was brought to 50 psi, and the hydrogenation was continued at 50° C. for 20 h. There was no more hydrogen gas consumed in the last 20 h. After cooling the reaction mixture to 20° C., the solid mixture, which contained Pd(10%)/C and product, was filtered. The solid mixture was suspended in DMSO (200 mL), and the suspension was heated on a hot plate to 80° C. inside with stirring for 10 min. The hot suspension was filtered and the Pd(10%)/C solid washed with a small portion of DMSO (50 mL). The DMSO filtrate and washing were combined and poured into water (600 mL). An off-white solid product precipitated out, and the suspension was stirred for 1 h before filtering and lyophilizing. The title compound was obtained as an off-white solid (11.3 g, >95% pure, 86% yield). ¹H NMR (300 MHz, DMSO-D₆) δ, ppm 3.84 (s, 3H) 6.68 (d, J=2.8 Hz, 1H) 7.73 (d, J=3.0 Hz, 1H) 8.36 (s, 1H) 8.80 (s, 1H) 11.99 (s, 1H). LCMS: (APCI, M−H⁻)=175.

(d) Methyl 1-(4-fluorobenzyl)-1H-pyrrolo[2,3-c]pyridine-5-carboxylate. To a stirred solution of methyl 1H-pyrrolo[2,3-c]pyridine-5-carboxylate (15.0 g, 85.1 mmol) in DMF (120 mL) at 10° C. under a nitrogen atmosphere was added sodium hydride (3.75 g, 60% in mineral oil, 93.7 mmol) in three portions over 5 min. The slurry became a homogeneous solution. After 130 min at 10° C., 4-fluorobenzyl bromide (0.60 g, 2.89 mmol) was added at such a rate that the temperature did not exceed 15° C. The resulting mixture was stirred for 2.5 hours at ambient temperature, quenched with water (120 mL), and extracted with ethyl acetate (3×30 mL). The combined organic extracts were washed with water (2×30 mL), dried over sodium sulfate, filtered, concentrated under reduced pressure and purified by flash chromatography. Elution with hexane:ethyl acetate (2:1) provided the title compound as a white solid (21.3 g, 88% yield). ¹H NMR (300 MHz, DMSO-D₆) δ, ppm: 3.84 (s, 3H), 5.59 (s, 2H), 6.73 (d, J=2.8 Hz, 1H), 7.15 (t, J=8.9 Hz, 2H), 7.34 (dd, J=8.3, 5.7 Hz, 2H), 7.87 (d, J=2.8 Hz, 1H), 8.3 (s, 1H), 8.97 (s, 1H). LCMS (APCI, M+H⁺): 285.3.

(e) Methyl 3-[(dimethylamino)methyl]-1-(4-fluorobenzyl)-1H-pyrrolo[2,3-c]pyridine-5-carboxylate. To a stirring solution of methyl 1-(4-fluorobenzyl)-1H-pyrrolo[2,3-c]pyridine-5-carboxylate (57.45 g, 0.202 mol) in acetonitrile (700 mL) was added N,N-dimethylmethylene ammonium chloride (Eschenmoser's salt, 37.8 g, 0.405 mol) and the solution was heated to reflux for approximately 1 h. The slurry was cooled and the white precipitate was filtered away. Saturated sodium bicarbonate solution was added to the white solid and the mixture was extracted with dichloromethane (3×200 mL). The combined organic extracts were dried over sodium sulfate, filtered and concentrated under reduced pressure to give a white solid (66.65 g, 97%) that was sufficiently pure to carry on to the next step crude.

(f) Methyl 3-{[(2S)-2-(aminocarbonyl)pyrrolidin-1-yl]methyl}-1-(4-fluorobenzyl)-1H-pyrrolo[2,3-c]pyridine-5-carboxylate. To a stirring solution of methyl 3-[(dimethylamino)methyl]-1-(4-fluorobenzyl)-1H-pyrrolo[2,3-c]pyridine-5-carboxylate (40.0 g, 117 mmol) in dichloromethane (400 mL) at room temperature was added ethyl chloroformate (11.15 mL, 117 mmol) and the mixture was stirred for 20 min. To this solution was added a solution of L-prolinamide (14.7 g, 129 mmol) and Hunig's base (61 mL, 351 mmol) in dimethylformamide (100 mL) dropwise and the mixture was stirred overnight at room temperature. Saturated sodium bicarbonate was added and the mixture was extracted with dichloromethane (3×500 mL). The combined organic extracts were dried over sodium sulfate, filitered and evaportated under reduced pressure to yield a crude off-whitish solid (65 g). The solid was dissolved in methanol (250 mL) and then precipitated out with the addition of water (620 mL). The white solid was filtered and dried (23 g, 48%). Additional material (approx. 3-5 g) can be obtained by purification of the mother liquor. ¹H NMR (300 MHz, CDCl₃) δ, ppm: 8.71 (1H, s), 8.51 (1H, s), 7.29-7.22 (1H, s), 7.13-7.09 (2H, m), 7.05-7.6.98 (2H, m), 6.91 (1H, m), 5.36 (2H, s), 4.99 (1H, bs), 4.01 (3H, s), 3.91 (2H, s), 3.17-3.10 (2H, m), 2.47 (1H, m), 2.21 (1H, m), 1.90 (1H, m), 1.79 (2H, m). LCMS (ESI, M+H⁺): 411.10.

(g) 3-{[(2S)-2-(aminocarbonyl)pyrrolidin-1-yl]methyl}-1-(4-fluorobenzyl)-1H-pyrrolo[2,3-c]pyridine-5-carboxylic acid. The crude methyl 3-{[(2S)-2-(aminocarbonyl)pyrrolidin-1-yl]methyl}-1-(4-fluorobenzyl)-1H-pyrrolo[2,3-c]pyridine-5-carboxylate (44.21 g, 107.83 mmol) was taken into methanol (600 mL). To this stirring solution was added 3 M LiOH_{(aq)} (79.08 mL, 2.2 eq), and the resulting mixture was stirred at room temperature for 24 h. The solution was acidified by the addition of conc. HCl (approx. 15 mL), until the pH measured 6-7. The volatiles were removed under reduced pressure, leaving an off-whitish solid. 500 mL of acetone was added and the slurry was stirred and then filtered to provide a white solid (43 g), which was sufficiently pure to carry to the next step.

(h) 3-{[(2S)-2-(aminocarbonyl)pyrrolidin-1-yl]methyl}-1-(4-fluorobenzyl)-N-hydroxy-N-methyl-1H-pyrrolo[2,3-c]pyridine-5-carboxamide. To a stirred solution of crude 3-{[(2S)-2-(aminocarbonyl)pyrrolidin-1-yl]methyl}-1-(4-fluorobenzyl)-1H-pyrrolo[2,3-c]pyridine-5-carboxylic acid (40.45 g, 102.15 mmol) in DMF (800 mL) was added N-methylmorpholine (13.48 mL, 122.58 mmol) followed by CDMT (21.52 g, 122.58 mmol) and the mixture was stirred at room temperature for 2 h using an overhead stirrer. When LCMS showed complete conversion to the activated acid, N-methylhydroxylamine hydrochloride (34.1 g, 408.60 mmol) was added and the mixture was stirred overnight. Saturated sodium bicarbonate was added and the mixture was extracted with ethyl acetate (3×200 mL). The combined organic extracts were dried over sodium sulfate, filtered and concentrated under reduced pressure. The off-whitish solid was crystallized from isopropanol/methanol using the following method. Isoppropanol (500 mL) was added and the mixture was heated to boil. Methanol was added slowly until the solid dissolved completely. The flask was set aside to cool to room temperature and crystals formed after 1 h, which were filtered to give a white solid (27 g, 62%). ¹H NMR (300 MHz, MeOH) δ, ppm: 8.66 (s, 1H) 8.29 (s, 1H) 7.69 (s, 1H) 7.23 (dd, J=8.40, 5.38 Hz, 2H) 7.05 (t, J=8.59 Hz, 2H) 5.51 (s, 2H) 3.93 (s, 2H) 3.42 (s, 3H) 3.12 (ddd, J=14.87, 9.87, 4.72 Hz, 2H) 2.51 (q, J=8.44 Hz, 1H) 2.13-2.26 (m, 1H) 1.75-1.85 (m, 3H). HRMS calcd for C₂₂H₂₃FN₅O₃ (M+H) 426.1941, found 426.1960.

### Example 43

### 3-{[(2R)-2-(aminocarbonyl)pyrrolidin-1-yl]methyl}-1-(4-fluorobenzyl)-N-hydroxy-N-methyl-1H -pyrrolo[2,3-c]pyridine-5-carboxamide

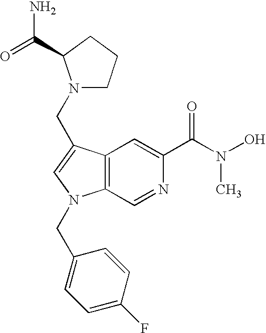

Example 43 was prepared in analogous fashion to example 42 except that D-prolinamide was used in place of L-prolinamide.

### Example 44

### Integrase Strand-Transfer Scintillation Proximity Assay Oligonucleotides

Oligonucleotide #1-5′-(biotin)CCCCTTTTAGTCAGTGTGGAAAATCTCTAGCA-3′ (SEQ ID NO: 1) and oligonucleotide #2-5′-ACTGCTAGAGATTTTCCACACTGACTAAAAG-3′ (SEQ ID NO: 2), were synthesized by TriLink BioTechnologies, Inc. (San Diego, Calif.). The annealed product represents preprocessed viral ds-DNA derived from the LTR U5 sequence of the viral genome. A ds-DNA control to test for non-specific interactions was made using a 3′ di-deoxy derivative of oligonucleotide #1 annealed to oligonucleotide #2. The CA overhang at the 5′ end of the non-biotinylated strand of the ds-DNA was created artificially by using a complimentary DNA oligonucleotide shortened by 2 base pairs. This configuration eliminates the requisite 3′ processing step of the integrase enzyme prior to the strand-transfer mechanism.

Host ds-DNA was prepared as an unlabeled and [³H]-thymidine labeled product from annealed oligonucleotide #3-5-AAAAAATGACCAAGGGCTAATTCACT-3′ (SEQ ID NO: 3), and oligonucleotide #4-5′-AAAAAAAGTGAATTAGCCCTTGGTCA-3′ (SEQ ID NO: 4), both synthesized by TriLink BioTechnologies, Inc. (San Diego, Calif.). The annealed product had overhanging 3′ ends of poly(dA). Host DNA was custom radiolabeled by PerkinElmer Life Sciences Inc. (Boston, Mass.) using an enzymatic method with a 12/1 ratio of [methyl-3H]dTTP/cold ds-DNA to yield 5′-blunt end ds-DNA with a specific activity of >900 Ci/mmol. The radiolabeled product was purified using a NENSORB cartridge and stored in stabilized aqueous solution (PerkinElmer). The final radiolabeled product had six [³H]-thymidine nucleotides at both 5′ ends of the host ds-DNA.

Reagents: Streptavidin-coated polyvinyltoluene (PVT) SPA beads were purchased from Amersham Biosciences (Piscataway, N.J.). Cesium chloride was purchased from Shelton Scientific, Inc. (Shelton, Conn.). White, polystyrene, flat-bottom, non-binding surface, 96-well plates were purchased from Corning. All other buffer components were purchased from Sigma (St. Louis, Mo.) unless otherwise indicated.

Enzyme Construction Full-length wild type HIV-1 integrase (SF1) sequence (amino acids 1-288) was constructed in a pET24a vector (Novagen, Madison, Wis.). The construct was confirmed through DNA sequencing.

Enzyme Purification Full length wild-type HIV Integrase was expressed in E. coli BL21 (DE3) cells and induced with 1 mM isopropyl-1 thio-β-D-galactopyranoside (IPTG) when cells reached an optical density between 0.8-1.0 at 600 nm. Cells were lysed by microfluidation in 50 mM HEPES pH 7.0, 75 mM NaCl, 5 mM DTT, 1 mM 4-(2-Aminoethyl)benzenesulfonylfluoride HCl (AEBSF). Lysate was then centrifuged 20 minutes at 11 k rpm in GSA rotor in Sorvall RC-5B at 4° C. Supernant was discarded and pellet resuspended in 50 mM HEPES pH 7.0, 750 mM NaCl, 5 mM DTT, 1 mM AEBSF and homogenized in a 40 mL Dounce homogenizer for 20 minutes on ice. Homogenate was then centrifuged 20 minutes at 11 k rpm in SS34 rotor in Sorvall RC-5B at 4° C. Supernant was discarded and pellet resuspended in 50 mM HEPES pH 7.0, 750 mM NaCl, 25 mM CHAPS, 5 mM DTT, 1 mM AEBSF. Preparation was then centrifuged 20 minutes at 11 k rpm in SS34 rotor in Sorvall RC-5B at 4° C.

Supernant was then diluted 1:1 with 50 mM HEPES pH 7.0, 25 mM CHAPS, 1 mM DTT, 1 mM AEBSF and loaded onto a Q-Sepharose column pre-equilibrated with 50 mM HEPES, pH 7.0, 375 mM NaCl, 25 mM CHAPS, 1 mM DTT, 1 mM AEBSF. The flow through peak was collected and NaCl diluted to 0.1 M with 50 mM HEPES pH 7.0, 25 mM CHAPS, 1 mM DTT, 0.5 mM AEBSF and loaded onto a SP-Sepharose column pre-equilibrated with 50 mM HEPES pH 7.0, 100 mM NaCl, 25 mM CHAPS, 1 mM DTT, 0.5 mM AEBSF. After washing the column with the equilibration buffer, a 100 to 400 mM NaCl gradient was run. The eluted integrase was concentrated and run on a S-300 gel diffusion column using 50 mM HEPES pH 7.0, 500 mM NaCl, 25 mM CHAPS, 1 mM DTT, 0.5 mM AEBSF. The peak from this column was concentrated to 0.76 mg/mL and stored at −70° C. and later used for strand transfer assays. All columns were run in a 4° C. cold room.

Viral DNA Bead Preparation: Streptavidin-coated SPA beads were suspended to 20 mg/mL in 25 mM 3-morpholinopropanesulfonic acid (MOPS) (pH 7.2) and 1.0% NaN₃. Biotinylated viral DNA was bound to the hydrated SPA beads in a batch process by combining 25 pmoles of ds-DNA to 1 mg of suspended SPA beads (10 μL of 50 μM viral DNA to 1 mL of 20 mg/mL SPA beads). The mixture was incubated at 22° C. for a minimum of 20 min. with occasional mixing followed by centrifugation at 2500 rpm for 10 min. However, the centrifugation speed and time may vary depending upon the particular centrifuge and conditions. The supernatant was removed and the beads suspended to 20 mg/mL in 25 mM MOPS (pH 7.2) and 1.0% NaN₃. The viral DNA beads were stable for several weeks when stored at 4° C. Di-deoxy viral DNA was prepared in an identical manner to yield control di-deoxy viral DNA beads.

Preparation of Integrase-DNA Complex: Assay buffer was made as a 10× stock of 250 mM MOPS (pH 7.2), 500 mM NaCl, 50 mM 3-[(3-cholamidopropyl)dimethylammonio]-1-propanesulfonate

(CHAPS), 0.5% (octylphenoxy)polyethoxyethanol (NP40) (IGEPAL-CA) and 0.05% NaN₃. Viral DNA beads were diluted to 2.67 mg/mL in 1× assay buffer plus 3 mM MgCl₂, 1% DMSO, and 10 mM fresh DTT. Integrase (IN) was pre-complexed to viral DNA beads in a batch process (IN/viral DNA/bead complex) by combining diluted viral DNA beads with integrase at a concentration of 385 nM followed by a minimum incubation time of 20 min. at 22° C. with gentle agitation. The sample was kept at 22° C. until transferred to the assay wells.

Preparation of Host DNA: Host DNA was prepared to 200 nM as a mixture of unlabeled and [³H]T-labeled host DNA diluted in 1× assay buffer plus 8.5 mM MgCl₂ and 15 mM DTT. Concentrations used were 4 nM [³H]T-labeled host DNA and 196 nM unlabeled host DNA. This ratio generates a SPA signal of 2000-3000 CPM in the absence of modulators such as inhibitors.

Strand-transfer Scintillation Proximity Assay: The strand-transfer reaction was carried out in 96-well microtiter plates, with a final enzymatic reaction volume of 100 μL. Ten microliters of compounds or test reagents diluted in 10% DMSO were added to the assay wells followed by the addition of 65 μL of the IN/viral-DNA/bead complex and mixed on a plate shaker. Then 25 μL of host DNA was added to the assay wells and mixed on a plate shaker. The strand-transfer reaction was initiated by transferring the assay plates to 37° C. dry block heaters. An incubation time of 50 min., which was shown to be within the linear range of the enzymatic reaction, was used. The final concentrations of integrase and host DNA in the assay wells were 246 nM and 50 nM, respectively.

The integrase strand-transfer reaction was terminated by adding 70 μL of stop buffer (150 mM EDTA, 90 mM NaOH, and 6 M CsCl) to the wells. Components of the stop buffer function to terminate enzymatic activity (EDTA), dissociate integrase/DNA complexes in addition to separating non-integrated DNA strands (NaOH), and float the SPA beads to the surface of the wells to be in closer range to the PMT detectors of the TopCount® plate-based scintillation counter (PerkinElmer Life Sciences Inc. (Boston, Mass.)). After the addition of stop buffer, the plates were mixed on a plate shaker, sealed with transparent tape, and allowed to incubate a minimum of 60 min. at 22° C. The assay signal was measured using a TopCount® plate-based scintillation counter with settings optimal for [³H]-PVT SPA beads. The TopCount® program incorporated a quench standardization curve to normalize data for color absorption of the compounds. Data values for quench-corrected counts per minute (QCPM) were used to quantify integrase activity. Counting time was 2 min./well.

The di-deoxy viral DNA beads were used to optimize the integrase strand-transfer reaction. The di-deoxy termination of the viral ds-DNA sequence prevented productive integration of viral DNA into the host DNA by integrase. Thus, the assay signal in the presence of di-deoxy viral DNA was a measure of non-specific interactions. Assay parameters were optimized to where reactions with di-deoxy viral DNA beads gave an assay signal closely matched to the true background of the assay. The true background of the assay was defined as a reaction with all assay components (viral DNA and [³H]-host DNA) in the absence of integrase.

Determination of Compound Activity: The percent inhibition of the compound was calculated using the equation (1−((QCPM sample−QCPM min)/(QCPM max−QCPM min)))*100. The min value is the assay signal in the presence of a known inhibitor at a concentration 100-fold higher than the IC₅₀ for that compound. The min signal approximates the true background for the assay. The max value is the assay signal obtained for the integrase-mediated activity in the absence of compound (i.e. with DMSO instead of compound in DMSO).

Compounds were prepared in 100% DMSO at 100-fold higher concentrations than desired for testing in assays (generally 5 mM), followed by dilution of the compounds in 100% DMSO to generate an 11-point titration curve with ½-log dilution intervals. The compound sample was further diluted 10-fold with water and transferred to the assay wells. The percentage inhibition for an inhibitory compound was determined as above with values applied to a nonlinear regression, sigmoidal dose response equation (variable slope) using GraphPad Prism curve fitting software (GraphPad Software, Inc., San Diego, Calif.). Concentration curves were assayed in duplicate and then repeated in an independent experiment.

### Example 45

### HIV-1 Cell Protection Assay

The antiviral activities of potential modulator compounds (test compounds) were determined in HIV-1 cell protection assays using the RF strain of HIV-1, CEM-SS cells, and the XTT dye reduction method (Weislow, O. S. et al., J. Natl. Cancer Inst. 81: 577-586 (1989)). Subject cells were infected with HIV-1 RF virus at an moi of 0.025 to 0.819 or mock infected with medium only and added at 2×10⁴ cells per well into 96 well plates containing half-log dilutions of test compounds. Six days later, 50 μl of XTT solution (1 mg/ml XTT tetrazolium and 0.02 nM phenazine methosulfate) were added to the wells and the plates were reincubated for four hours. Viability, as determined by the amount of XTT formazan produced, was quantified spectrophotometrically by absorbance at 450 nm.

Data from CPE assays were expressed as the percent of formazan produced in compound-treated cells compared to formazan produced in wells of uninfected, compound-free cells. The fifty percent effective concentration (EC₅₀) was calculated as the concentration of compound that affected an increase in the percentage of formazan production in infected, compound-treated cells to 50% of that produced by uninfected, compound-free cells. The 50% cytotoxicity concentration (CC₅₀) was calculated as the concentration of compound that decreased the percentage of formazan produced in uninfected, compound-treated cells to 50% of that produced in uninfected, compound-free cells. The therapeutic index was calculated by dividing the cytotoxicity (CC₅₀) by the antiviral activity (EC₅₀).

1. A compound of formula (I), 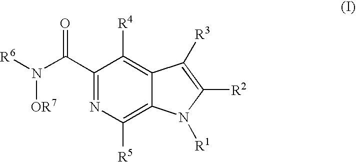
wherein:
R¹ is hydrogen, C₁-C₈ alkyl, C₂-C₈ alkenyl, or C₁-C₈ heteroalkyl, wherein said C₁-C₈ alkyl, C₂-C₈ alkenyl, or C₁-C₈ heteroalkyl groups may be optionally substituted with one or more substituent independently selected from:
halo, —OR^{15a}, —N(R^{15a}R^{15b})_{b}, —C(O)N(R^{15a}R^{15b}), —NR^{15a}C(O)N(R^{15a}R^{15b}), —NR^{15a}C(O)R^{15a}, —NR^{15a}C(NR^{15a})N(R^{15a}R^{15b}), —SR^{15a}, —S(O)R^{15a}, —S(O)₂R^{15a}, —S(O)₂N(R^{15a}R^{15b}), C₁-C₈ alkyl, C₆-C₁₄ aryl, C₃-C₈ cycloalkyl, and C₂-C₉ heteroaryl, wherein said C₁-C₈ alkyl, C₆-C₁₄ aryl, C₃-C₈ cycloalkyl, and C₂-C₉ heteroaryl groups are optionally substituted with one or more substituent independently selected from halo, —C(R^{15a}R^{15b}R^{15c}), —OH, and C₁-C₈ alkoxy;
R² is hydrogen;
R³ is —(CR⁸R⁹)ₜNR¹⁰R¹¹ or —(CR⁸R⁹)ₜN(R^{15a}R¹⁶);
R⁴ is hydrogen, halo, C₁-C₈ alkyl, —OR^{15a}, —NR^{15a}R^{15b}, C₁-C₈ heteroalkyl, C₂-C₈ alkenyl, or C₂-C₈ alkynyl, wherein said C₂-C₈ alkenyl or C₂-C₈ alkynyl are optionally substituted with one or more R¹² group;
R⁵ is hydrogen;
R⁶ is hydrogen, C₁-C₈ alkyl, C₁-C₈ heteroalkyl, or C₂-C₈ alkenyl, wherein said C₁-C₈ alkyl and C₂-C₈ alkenyl groups are optionally substituted with one or more C₆-C₁₄ aryl or —OR^{15a} group;
R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with one or more C₃-C₈ cycloalkyl or C₆-C₁₄ aryl group;
each R³ and R⁹, which may be the same or different, are independently selected from hydrogen and C₁-C₈ alkyl;
R¹⁰ and R¹¹, together with the nitrogen atom to which they are attached, form a C₂-C₉ heterocyclyl group, substituted with at least one R¹³ group;
each R¹² is independently selected from —OR^{15a}, halo, C₆-C₁₄ aryl, C₂-C₉ heteroaryl, C₁-C₈ heteroalkyl, C₃-C₈ cycloalkyl, C₂-C₉ heterocyclyl, and —C(R^{15a}R^{15b}R^{15c});
R¹³ is selected from —(CR⁸R⁹)ₜ—OR^{15a}, —(CR⁸R⁹)ₜ—C(O)R^{15a}, —(CR⁸R⁹)ₜ—C(O)NR^{15a}R^{15b}, —(CR⁸R⁹)ₜ—S—R^{15a}, —(CR⁸R⁹)ₜ—S(O)—R^{15a}, —(CR⁸R⁹)ₜ—S(O)₂—R^{15a}, —(CR⁸R⁹)ₜ—(C₂-C₉ heterocyclyl), —(CR⁸R⁹)ₜ—(C₆-C₁₄ aryl) and —(CR⁸R⁹)ₜ—(C₂-C₉ heteroaryl);
each R^{15a}, R^{15b}, and R^{15c}, which may be the same or different, is independently selected from hydrogen and C₁-C₈ alkyl;
R¹⁶ is —(CH₂)ₘ—(C₂-C₉ heterocyclyl) or —(CH₂)ₘ—(C₃-C₈ cycloalkyl), wherein said C₂-C₉ heterocyclyl and C₃-C₈ cycloalkyl groups are substituted with one or more groups selected from C₃-C₈ cycloalkyl and —(CR⁸R⁹)ₜ—OR¹⁵;
each m is independently selected from 0, 1, and 2; and
each t is independently selected from 0, 1, 2, and 3; or
a pharmaceutically acceptable salt or solvate thereof.

2. A compound according to claim 1, wherein R³ is —(CR⁸R⁹)ₜNR¹⁰R¹¹ and R⁴ is hydrogen.

3. A compound according to claim 2, wherein R¹ is C₁-C₈ alkyl substituted with C₆-C₁₄ aryl, wherein said C₆-C₁₄ aryl group is optionally substituted with one or more substituent independently selected from halo, —C(R^{15a}R^{15b}R^{15c}), —OH, and C₁-C₈ alkoxy.

4. A compound according to claim 3, wherein:
R¹ is C₁-C₈ alkyl substituted with C₆-C₁₄ aryl, wherein said C₆-C₁₄ aryl group is substituted with one or more halo;
R⁶ is hydrogen or C₁-C₈ alkyl; and
R⁷ is hydrogen or C₁-C₈ alkyl.

5. A compound according to claim 4, wherein R¹ is C₁-C₈ alkyl substituted with C₆-C₁₄ aryl, wherein said C₆-C₁₄ aryl group is substituted with one or more fluorine.

6. A compound according to claim 5, wherein:
R¹ is 4-fluorobenzyl or 2,4-difluorobenzyl;
R⁶ is hydrogen or —CH₃;
R⁷ is hydrogen; and
R¹³ is selected from —OR^{15a}, —C(O)R^{15a}, —C(O)NR^{15a}R^{15b}, —S—R^{15a}, —S(O)—R^{15a}, —S(O)₂—R^{15a}, C₂-C₉ heterocyclyl, C₆-C₁₄ aryl and C₂-C₉ heteroaryl.

7. A compound according to claim 6, wherein R¹³ is selected from —OH, —C(O)CH₃, —C(O)NH₂, —S(O)₂CH₃, C₂-C₉ heterocyclyl, C₆-C₁₄ aryl and C₂-C₉ heteroaryl.

8. A compound according to claim 7, wherein R¹³ is selected from —OH, —C(O)CH₃, —C(O)NH₂, and —S(O)₂CH₃.

9. A compound according to claim 1, wherein R³ is —(CR⁸R⁹)ₜN(R^{15a}R¹⁶).

10. A compound according to claim 9, wherein:
R¹ is C₁-C₈ alkyl substituted with C₆-C₁₄ aryl wherein said C₆-C₁₄ aryl is optionally substituted with one or more halo;
R⁴ is hydrogen;
R⁶ is hydrogen or C₁-C₈ alkyl;
R⁷ is hydrogen or C₁-C₈ alkyl; and
each R^{15a}, which may be the same or different, is independently selected from hydrogen and C₁-C₈ alkyl.

11. A compound according to claim 10, wherein R¹ is C₁-C₈ alkyl substituted with C₆-C₁₄ aryl wherein said C₆-C₁₄ aryl is optionally substituted with one or more fluorine.

12. A compound according to claim 1, selected from:
1-(2,4-difluorobenzyl)-N-hydroxy-3-{[3-(methylsulfonyl)pyrrolidin-1-yl]methyl}-1H-pyrrolo[2,3-c]pyridine-5-carboxamide;
3-{[(2S)-2-(aminocarbonyl)pyrrolidin-1-yl]methyl}-1-(2,4-difluorobenzyl)-N-hydroxy-1H-pyrrolo[2,3-c]pyridine-5-carboxamide;
1-(2,4-difluorobenzyl)-N-hydroxy-3-[(4-pyridin-2-yl piperazin-1-yl)methyl]-1H-pyrrolo[2,3-c]pyridine-5-carboxamide;
1-(2,4-difluorobenzyl)-3-(3,4-dihydroisoquinolin-2(1H)-ylmethyl)-N-hydroxy-1H-pyrrolo[2,3-c]pyridine-5-carboxamide;
3-{[4-(aminocarbonyl)piperidin-1-yl]methyl}-1-(2,4-difluorobenzyl)-N-hydroxy-1H-pyrrolo[2,3-c]pyridine-5-carboxamide;
1-(2,4-difluorobenzyl)-N-hydroxy-3-[(3-hydroxypyrrolidin-1-yl)methyl]-1H-pyrrolo[2,3-c]pyridine-5-carboxamide;
3-[(4-acetylpiperazin-1-yl)methyl]-1-(2,4-difluorobenzyl)-N-hydroxy-1H-pyrrolo[2,3-c]pyridine-5-carboxamide;
1-(2,4-difluorobenzyl)-N-hydroxy-3-[(4-hydroxypiperidin-1-yl)methyl]-1H-pyrrolo[2,3-c]pyridine-5-carboxamide;
3-{[3-(aminocarbonyl)piperidin-1-yl]methyl}-1-(4-fluorobenzyl)-N-hydroxy-1H-pyrrolo[2,3-c]pyridine-5-carboxamide;
3-{[(2S)-2-(aminocarbonyl)pyrrolidin-1-yl]methyl}-1-(4-fluorobenzyl)-N-hydroxy-N-methyl-1H-pyrrolo[2,3-c]pyridine-5-carboxamide;
3-{[(2S)-2-(aminocarbonyl)pyrrolidin-1-yl]methyl}-1-(4-fluorobenzyl)-N-hydroxy-1H-pyrrolo[2,3-c]pyridine-5-carboxamide;
1-(4-fluorobenzyl)-N,4-dihydroxy-1H-pyrrolo[2,3-c]pyridine-5-carboxamide;
1-(4-fluorobenzyl)-N-hydroxy-4-methoxy-N-methyl-1H-pyrrolo[2,3-c]pyridine-5-carboxamide;
3-{[(2S)-2-(aminocarbonyl)pyrrolidin-1-yl]methyl}-1-(2,4-difluorobenzyl)-N-hydroxy-N-methyl-1H-pyrrolo[2,3-c]pyridine-5-carboxamide;
1-(4-fluorobenzyl)-N-hydroxy-3-{[3-(methylsulfonyl)pyrrolidin-1-yl]methyl}-1H-pyrrolo[2,3-c]pyridine-5-carboxamide;
1-(4-fluorobenzyl)-N-hydroxy-3-[(4-hydroxypiperidin-1-yl)methyl]-1H-pyrrolo[2,3-c]pyridine-5-carboxamide;
1-(4-fluorobenzyl)-N-hydroxy-3-[(4-hydroxypiperidin-1-yl)methyl]-N-methyl-1H-pyrrolo[2,3-c]pyridine-5-carboxamide;
1-(4-fluorobenzyl)-N-hydroxy-3-[(3-hydroxypyrrolidin-1-yl)methyl]-N-methyl-1H-pyrrolo[2,3-c]pyridine-5-carboxamide;
1-(4-fluorobenzyl)-N-hydroxy-N-methyl-3-{[3-(methylsulfonyl)pyrrolidin-1-yl]methyl}-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-{[(7R,8aS)-7-hydroxyhexahydropyrrolo[1,2-a]pyrazin-2(1H)-yl]methyl}-1H-pyrrolo[2,3-c]pyridine-5-carboxamide;
3-{[[2-(1-cyclopropyl-5-oxopyrrolidin-2-yl)ethyl](methyl)amino]methyl}-1-(2,4-difluorobenzyl)-N-hydroxy-1H-pyrrolo[2,3-c]pyridine-5-carboxamide;
1-(2,4-difluorobenzyl)-N-hydroxy-3-({[1-(hydroxymethyl)cyclopentyl]amino}methyl)-1H-pyrrolo[2,3-c]pyridine-5-carboxamide;
1-(2,4-difluorobenzyl)-N-hydroxy-3-({[1-(hydroxymethyl)cyclopentyl]amino}methyl)-N-methyl-1H-pyrrolo[2,3-c]pyridine-5-carboxamide;
1-(2,4-difluorobenzyl)-3-({[1-(hydroxymethyl)cyclopentyl]amino}methyl)-N-methoxy-1H-pyrrolo[2,3-c]pyridine-5-carboxamide;
3-{[[2-(1-cyclopropyl-5-oxopyrrolidin-2-yl)ethyl](methyl)amino]methyl}-1-(2,4-difluorobenzyl)-N-hydroxy-N-methyl-1H-pyrrolo[2,3-c]pyridine-5-carboxamide;
3-{[[2-(1-cyclopropyl-5-oxopyrrolidin-2-yl)ethyl](methyl)amino]methyl}-1-(2,4-difluorobenzyl)-N-methoxy-1H -pyrrolo[2,3-c]pyridine-5-carboxamide;
3-{[3-(aminocarbonyl)piperidin-1-yl]methyl}-1-(4-fluorobenzyl)-N-hydroxy-N-methyl-1H-pyrrolo[2,3-c]pyridine-5-carboxamide;
1-(2,4-difluorobenzyl)-3-({4-hydroxy-4-[(2-oxopyrrolidin-1-yl)methyl]piperidin-1-yl}methyl)-N-methoxy-1H-pyrrolo[2,3-c]pyridine-5-carboxamide;
1-(2,4-difluorobenzyl)-N-ethyl-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-propyl-1H-pyrrolo[2,3-c]pyridine-5-carboxamide;
N-benzyl-1-(2,4-difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-methyl-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-(3-hydroxypropyl)-1H-pyrrolo[2,3-c]pyridine-5-carboxamide;
1-(2,4-difluorobenzyl)-N-ethoxy-3-({4-hydroxy-4-[(2-oxopyrrolidin-1-yl)methyl]piperidin-1-yl}methyl)-1H-pyrrolo[2,3-c]pyridine-5-carboxamide;
N-(benzyloxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide;
N-(cyclopropylmethoxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-3-({4-hydroxy-4-[(2-oxopyrrolidin-1-yl)methyl]piperidin-1-yl}methyl)-N-phenoxy-1H-pyrrolo[2,3-c]pyridine-5-carboxamide; and
1-(4-fluorobenzyl)-4-hydroxy-N-methoxy-1H-pyrrolo[2,3-c]pyridine-5-carboxamide; or
a pharmaceutically acceptable salt or solvate thereof.

13. A compound according to claim 1, selected from:
1-(2,4-difluorobenzyl)-N-hydroxy-3-{[3-(methylsulfonyl)pyrrolidin-1-yl]methyl}-1H-pyrrolo[2,3-c]pyridine-5-carboxamide;
3-{[(2S)-2-(aminocarbonyl)pyrrolidin-1-yl]methyl}-1-(2,4-difluorobenzyl)-N-hydroxy-1H-pyrrolo[2,3-c]pyridine-5-carboxamide;
1-(2,4-difluorobenzyl)-N-hydroxy-3-[(4-pyridin-2-yl piperazin-1-yl)methyl]-1H-pyrrolo[2,3-c]pyridine-5-carboxamide;
1-(2,4-difluorobenzyl)-3-(3,4-dihydroisoquinolin-2(1H)-ylmethyl)-N-hydroxy-1H-pyrrolo[2,3-c]pyridine-5-carboxamide;
3-{[4-(aminocarbonyl)piperidin-1-yl]methyl}-1-(2,4-difluorobenzyl)-N-hydroxy-1H-pyrrolo[2,3-c]pyridine-5-carboxamide;
1-(2,4-difluorobenzyl)-N-hydroxy-3-[(3-hydroxypyrrolidin-1-yl)methyl]-1H-pyrrolo[2,3-c]pyridine-5-carboxamide;
3-[(4-acetylpiperazin-1-yl)methyl]-1-(2,4-difluorobenzyl)-N-hydroxy-1H-pyrrolo[2,3-c]pyridine-5-carboxamide;
1-(2,4-difluorobenzyl)-N-hydroxy-3-[(4-hydroxypiperidin-1-yl)methyl]-1H-pyrrolo[2,3-c]pyridine-5-carboxamide;
3-{[3-(aminocarbonyl)piperidin-1-yl]methyl}-1-(4-fluorobenzyl)-N-hydroxy-1H-pyrrolo[2,3-c]pyridine-5-carboxamide;
3-{[(2S)-2-(aminocarbonyl)pyrrolidin-1-yl]methyl}-1-(4-fluorobenzyl)-N-hydroxy-N-methyl-1H-pyrrolo[2,3-c]pyridine-5-carboxamide;
3-{[(2S)-2-(aminocarbonyl)pyrrolidin-1-yl]methyl}-1-(4-fluorobenzyl)-N-hydroxy-1H-pyrrolo[2,3-c]pyridine-5-carboxamide;
3-{[(2S)-2-(aminocarbonyl)pyrrolidin-1-yl]methyl}-1-(2,4-difluorobenzyl)-N-hydroxy-N-methyl-1H-pyrrolo[2,3-c]pyridine-5-carboxamide;
1-(4-fluorobenzyl)-N-hydroxy-3-{[3-(methylsulfonyl)pyrrolidin-1-yl]methyl}-1H-pyrrolo[2,3-c]pyridine-5-carboxamide;
1-(4-fluorobenzyl)-N-hydroxy-3-[(4-hydroxypiperidin-1-yl)methyl]-1H-pyrrolo[2,3-c]pyridine-5-carboxamide;
1-(4-fluorobenzyl)-N-hydroxy-3-[(4-hydroxypiperidin-1-yl)methyl]-N-methyl-1H-pyrrolo[2,3-c]pyridine-5-carboxamide;
1-(4-fluorobenzyl)-N-hydroxy-3-[(3-hydroxypyrrolidin-1-yl)methyl]-N-methyl-1H-pyrrolo[2,3-c]pyridine-5-carboxamide;
1-(4-fluorobenzyl)-N-hydroxy-N-methyl-3-{[3-(methylsulfonyl)pyrrolidin-1-yl]methyl}-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-{[(7R,8aS)-7-hydroxyhexahydropyrrolo[1,2-a]pyrazin-2(1H)-yl]methyl}-1H-pyrrolo[2,3-c]pyridine-5-carboxamide;
3-{[3-(aminocarbonyl)piperidin-1-yl]methyl}-1-(4-fluorobenzyl)-N-hydroxy-N-methyl-1H-pyrrolo[2,3-c]pyridine-5-carboxamide;
1-(2,4-difluorobenzyl)-3-({4-hydroxy-4-[(2-oxopyrrolidin-1-yl)methyl]piperidin-1-yl}methyl)-N-methoxy-1H-pyrrolo[2,3-c]pyridine-5-carboxamide;
1-(2,4-difluorobenzyl)-N-ethyl-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-propyl-1H-pyrrolo[2,3-c]pyridine-5-carboxamide;
N-benzyl-1-(2,4-difluorobenzyl)-N-hydroxy-3-({4-hydroxy-4-[(2-oxopyrrolidin-1-yl)methyl]piperidin-1-yl}methyl)-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-methyl-1H-pyrrolo[2,3-c]pyridine-5-carboxamide;
1-(2,4-difluorobenzyl)-N-hydroxy-3-({4-hydroxy-4-[(2-oxopyrrolidin-1-yl)methyl]piperidin-1-yl}methyl)-N-(3-hydroxypropyl)-1H-pyrrolo[2,3-c]pyridine-5-carboxamide;
1-(2,4-difluorobenzyl)-N-ethoxy-3-({4-hydroxy-4-[(2-oxopyrrolidin-1-yl)methyl]piperidin-1-yl}methyl)-1H-pyrrolo[2,3-c]pyridine-5-carboxamide;
N-(benzyloxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide;
N-(cyclopropylmethoxy)-1-(2,4-difluorobenzyl)-3-({4-hydroxy-4-[(2-oxopyrrolidin-1-yl)methyl]piperidin-1-yl}methyl)-1H-pyrrolo[2,3-c]pyridine-5-carboxamide; and
1-(2,4-difluorobenzyl)-3-({4-hydroxy-4-[(2-oxopyrrolidin-1-yl)methyl]piperidin-1-yl}methyl)-N-phenoxy-1H-pyrrolo[2,3-c]pyridine-5-carboxamide; or
a pharmaceutically acceptable salt or solvate thereof.

14. A compound according to claim 1, selected from:
3-{[[2-(1-cyclopropyl-5-oxopyrrolidin-2-yl)ethyl](methyl)amino]methyl}-1-(2,4-difluorobenzyl)-N-hydroxy-1H-pyrrolo[2,3-c]pyridine-5-carboxamide;
1-(2,4-difluorobenzyl)-N-hydroxy-3-({[1-(hydroxymethyl)cyclopentyl]amino}methyl)-1H-pyrrolo[2,3-c]pyridine-5-carboxamide;
1-(2,4-difluorobenzyl)-N-hydroxy-3-({[1-(hydroxymethyl)cyclopentyl]amino}methyl)-N-methyl-1H-pyrrolo[2,3-c]pyridine-5-carboxamide;
1-(2,4-difluorobenzyl)-3-({[1-(hydroxymethyl)cyclopentyl]amino}methyl)-N-methoxy-1H-pyrrolo[2,3-c]pyridine-5-carboxamide;
3-{[[2-(1-cyclopropyl-5-oxopyrrolidin-2-yl)ethyl](methyl)amino]methyl}-1-(2,4-difluorobenzyl)-N-hydroxy-N-methyl-1H-pyrrolo[2,3-c]pyridine-5-carboxamide; and
3-{[[2-(1-cyclopropyl-5-oxopyrrolidin-2-yl)ethyl](methyl)amino]methyl}-1-(2,4-difluorobenzyl)-N-methoxy-1H -pyrrolo[2,3-c]pyridine-5-carboxamide; or
a pharmaceutically acceptable salt or solvate thereof.

15. A pharmaceutical composition for the treatment of HIV infection in an HIV infected mammal, comprising a therapeutically effective amount of at least one compound according to claim 1, or a pharmaceutically acceptable salt or solvate thereof, and a pharmaceutically acceptable carrier or diluent.

16. (canceled)

17. (canceled)

18. A method of treating acquired immune deficiency syndrome in an HIV infected mammal, comprising administering to said mammal a therapeutically effective amount of at least one compound according to claim 1, or a pharmaceutically acceptable salt or solvate thereof.

19. A method of inhibiting HIV replication in a cell, comprising contacting said cell with an HIV-inhibiting amount of at least one compound according to claim 1, or a pharmaceutically acceptable salt or solvate thereof.
